SEQUENCE LISTING (169 sequences)

SEQ ID NO: 1 - protein, 447 aa, synthetic construct
QVQLQQPGAELVKPGASVKLSCKASGYAFTSYWMHWVKLRPGQGFEWIGEIYPTNGGTTYNEKFKRKATLTVDKSSSTAYMQLSSLTSEDSAVYYCTIEDFYAMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 2 - protein, 117 aa, synthetic construct
QVQLQQPGAELVKPGASVKLSCKASGYAFTSYWMHWVKLRPGQGFEWIGEIYPTNGGTTYNEKFKRKATLTVDKSSSTAYMQLSSLTSEDSAVYYCTIEDFYAMDYWGQGTSVTVSS

SEQ ID NO: 3 - protein, 5 aa, synthetic construct
SYWMH

SEQ ID NO: 4 - protein, 17 aa, synthetic construct
EIYPTNGGTTYNEKFKR

SEQ ID NO: 5 - protein, 8 aa, synthetic construct
EDFYAMDY

SEQ ID NO: 6 - protein, 10 aa, synthetic construct
GYAFTSYWMH

SEQ ID NO: 7 - protein, 10 aa, synthetic construct
EIYPTNGGTT

SEQ ID NO: 8 - protein, 8 aa, synthetic construct
EDFYAMDY

SEQ ID NO: 9 - protein, 219 aa, synthetic construct
DVVMTQTPLTLSVTIGQPASISCKSSQSLLDSDGKTCLNWLLQRPGQSPKRLIYLVSKLDSGVPDRFTGSGSGTDFTLKISRVEAEDLGVYYCWQGTHFPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 10 - protein, 113 aa, synthetic construct
DVVMTQTPLTLSVTIGQPASISCKSSQSLLDSDGKTCLNWLLQRPGQSPKRLIYLVSKLDSGVPDRFTGSGSGTDFTLKISRVEAEDLGVYYCWQGTHFPYTFGGGTKLEIKR

SEQ ID NO: 11 - protein, 16 aa, synthetic construct
KSSQSLLDSDGKTCLN

SEQ ID NO: 12 - protein, 7 aa, synthetic construct
LVSKLDS

SEQ ID NO: 13 - protein, 9 aa, synthetic construct
WQGTHFPYT

SEQ ID NO: 14 - protein, 16 aa, synthetic construct
KSSQSLLDSDGKTCLN

SEQ ID NO: 15 - protein, 7 aa, synthetic construct
LVSKLDS

SEQ ID NO: 16 - protein, 9 aa, synthetic construct
WQGTHFPYT

SEQ ID NO: 17 - protein, 447 aa, synthetic construct
QVQLQQPGAELVKPGASVKLSCKASGYAFTSYWMHWVKLRPGQGFEWIGEIYPTNGGTTYNEKFKRKATLTVDKSSSTAYMQLSSLTSEDSAVYYCTIEDFYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 219 aa, synthetic construct
DVVMTQTPLTLSVTIGQPASISCKSSQSLLDSDGKTCLNWLLQRPGQSPKRLIYLVSKLDSGVPDRFTGSGSGTDFTLKISRVEAEDLGVYYCWQGTHFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 446 aa, synthetic construct
QVQLVQSGAEVKKPGASVKVSCKASGYAFTSYWMHWVRQAPGQRLEWMGEIYPTNGGTTYAQKFQGRVTMTRDTSTSTVYMELSRLRSDDTAVYYCAREDFYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 117 aa, synthetic construct
QVQLVQSGAEVKKPGASVKVSCKASGYAFTSYWMHWVRQAPGQRLEWMGEIYPTNGGTTYAQKFQGRVTMTRDTSTSTVYMELSRLRSDDTAVYYCAREDFYAMDYWGQGTTVTVSS

SEQ ID NO: 21 - protein, 17 aa, synthetic construct
EIYPTNGGTTYAQKFQG

SEQ ID NO: 22 - protein, 446 aa, synthetic construct
QVQLVQSGAEVKKPGASVKVSCKASGYAFTSYWMHWVRQAPGQRLEWMGEIYPTNGGTTYAQKFQGRVTMTVDKSTSTAYMELSRLRSDDTAVYYCTIEDFYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 117 aa, synthetic construct
QVQLVQSGAEVKKPGASVKVSCKASGYAFTSYWMHWVRQAPGQRLEWMGEIYPTNGGTTYAQKFQGRVTMTVDKSTSTAYMELSRLRSDDTAVYYCTIEDFYAMDYWGQGTTVTVSS

SEQ ID NO: 24 - protein, 446 aa, synthetic construct
QVQLVQSGAEVKKPGASVKLSCKASGYAFTSYWMHWVRQAPGQRLEWIGEIYPTNGGTTYAQKFQGRATLTVDKSTSTAYMELSRLRSDDTAVYYCTIEDFYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 117 aa, synthetic construct
QVQLVQSGAEVKKPGASVKLSCKASGYAFTSYWMHWVRQAPGQRLEWIGEIYPTNGGTTYAQKFQGRATLTVDKSTSTAYMELSRLRSDDTAVYYCTIEDFYAMDYWGQGTTVTVSS

SEQ ID NO: 26 - protein, 446 aa, synthetic construct
QVQLVQSGAEVKKPGASVKVSCKASGYAFTSYWMHWVRQAPGQRLEWMGEIYPTNGGTTYNEKFKRRVTMTVDKSTSTAYMELSRLRSDDTAVYYCTIEDFYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 117 aa, synthetic construct
QVQLVQSGAEVKKPGASVKVSCKASGYAFTSYWMHWVRQAPGQRLEWMGEIYPTNGGTTYNEKFKRRVTMTVDKSTSTAYMELSRLRSDDTAVYYCTIEDFYAMDYWGQGTTVTVSS

SEQ ID NO: 28 - protein, 446 aa, synthetic construct
QVQLVQSGAEVKKPGASVKLSCKASGYAFTSYWMHWVRQAPGQRLEWIGEIYPTNGGTTYNEKFKRRATLTVDKSTSTAYMELSRLRSDDTAVYYCTIEDFYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 117 aa, synthetic construct
QVQLVQSGAEVKKPGASVKLSCKASGYAFTSYWMHWVRQAPGQRLEWIGEIYPTNGGTTYNEKFKRRATLTVDKSTSTAYMELSRLRSDDTAVYYCTIEDFYAMDYWGQGTTVTVSS

SEQ ID NO: 30 - protein, 219 aa, synthetic construct
DIVMTQTPLSLSVTPGQPASISCKSSQSLLDSDGKTSLNWYLQKPGQSPQLLIYLVSKLDSGVPDRFSGSGSGTDFTLKISRVEAEDVGVYYCWQGTHFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 112 aa, synthetic construct
DIVMTQTPLSLSVTPGQPASISCKSSQSLLDSDGKTSLNWYLQKPGQSPQLLIYLVSKLDSGVPDRFSGSGSGTDFTLKISRVEAEDVGVYYCWQGTHFPYTFGQGTKLEIK

SEQ ID NO: 32 - protein, 16 aa, synthetic construct
KSSQSLLDSDGKTSLN

SEQ ID NO: 33 - protein, 17 aa, synthetic construct
IYSGNGDTSYNQKFKGK

SEQ ID NO: 34 - protein, 16 aa, synthetic construct
KSSQSLLDSDGKTSLN

SEQ ID NO: 35 - protein, 219 aa, synthetic construct
DIVMTQTPLSLSVTPGQPASISCKSSQSLLDSDGKTSLNWLLQKPGQSPQLLIYLVSKLDSGVPDRFSGSGSGTDFTLKISRVEAEDVGVYYCWQGTHFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 112 aa, synthetic construct
DIVMTQTPLSLSVTPGQPASISCKSSQSLLDSDGKTSLNWLLQKPGQSPQLLIYLVSKLDSGVPDRFSGSGSGTDFTLKISRVEAEDVGVYYCWQGTHFPYTFGQGTKLEIK

SEQ ID NO: 37 - protein, 219 aa, synthetic construct
DIVMTQTPLSLSVTPGQPASISCKSSQSLLDSDGKTSLNWLLQKPGQSPQRLIYLVSKLDSGVPDRFSGSGSGTDFTLKISRVEAEDVGVYYCWQGTHFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 112 aa, synthetic construct
DIVMTQTPLSLSVTPGQPASISCKSSQSLLDSDGKTSLNWLLQKPGQSPQRLIYLVSKLDSGVPDRFSGSGSGTDFTLKISRVEAEDVGVYYCWQGTHFPYTFGQGTKLEIK

SEQ ID NO: 39 - protein, 447 aa, synthetic construct
QVQLQQPGAELVKPGASVKMSCKASGYTFTSYNMHWVKQTPGQGLEWIGVIYSGNGDTSYNQKFKGKATLTADKSSSTAYMQINSLTSEDSAVYYCARERDTRFGNWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 40 - protein, 117 aa, synthetic construct
QVQLQQPGAELVKPGASVKMSCKASGYTFTSYNMHWVKQTPGQGLEWIGVIYSGNGDTSYNQKFKGKATLTADKSSSTAYMQINSLTSEDSAVYYCARERDTRFGNWGQGTLVTVSA

SEQ ID NO: 41 - protein, 5 aa, synthetic construct
SYNMH

SEQ ID NO: 42 - protein, 17 aa, synthetic construct
VIYSGNGDTSYNQKFKG

SEQ ID NO: 43 - protein, 8 aa, synthetic construct
ERDTRFGN

SEQ ID NO: 44 - protein, 218 aa, synthetic construct
NIVLTQSPASLAVSLGLRATISCRASESVDIYGNSFMHWYQQKPGQPPKLLIYLASNLESGVPARFSGSGSRTDFTLTIDPVEADDAATYYCQQNNEDPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 45 - protein, 111 aa, synthetic construct
NIVLTQSPASLAVSLGLRATISCRASESVDIYGNSFMHWYQQKPGQPPKLLIYLASNLESGVPARFSGSGSRTDFTLTIDPVEADDAATYYCQQNNEDPYTFGGGTKLEIK

SEQ ID NO: 46 - protein, 15 aa, synthetic construct
RASESVDIYGNSFMH

SEQ ID NO: 47 - protein, 7 aa, synthetic construct
LASNLES

SEQ ID NO: 48 - protein, 9 aa, synthetic construct
QQNNEDPYT

SEQ ID NO: 49 - protein, 447 aa, synthetic construct
QVQLQQPGAELVKPGASVKMSCKASGYTFTSYNMHWVKQTPGQGLEWIGVIYSGNGDTSYNQKFKGKATLTADKSSSTAYMQINSLTSEDSAVYYCARERDTRFGNWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 218 aa, synthetic construct
NIVLTQSPASLAVSLGLRATISCRASESVDIYGNSFMHWYQQKPGQPPKLLIYLASNLESGVPARFSGSGSRTDFTLTIDPVEADDAATYYCQQNNEDPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 447 aa, synthetic construct
QVQLVQSGAEVKKPGASVKVSCKASGYTFTSYNMHWVRQAPGQGLEWMGVIYSGNGDTSYAQKFQGRVTMTADTSTSTAYMELSSLRSEDTAVYYCARERDTRFGNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 117 aa, synthetic construct
QVQLVQSGAEVKKPGASVKVSCKASGYTFTSYNMHWVRQAPGQGLEWMGVIYSGNGDTSYAQKFQGRVTMTADTSTSTAYMELSSLRSEDTAVYYCARERDTRFGNWGQGTLVTVSS

SEQ ID NO: 53 - protein, 17 aa, synthetic construct
VIYSGNGDTSYAQKFQG

SEQ ID NO: 54 - protein, 447 aa, synthetic construct
QVQLVQSGAEVKKPGASVKMSCKASGYTFTSYNMHWVRQAPGQGLEWIGVIYSGNGDTSYAQKFQGRATLTADTSTSTAYMELSSLRSEDTAVYYCARERDTRFGNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 117 aa, synthetic construct
QVQLVQSGAEVKKPGASVKMSCKASGYTFTSYNMHWVRQAPGQGLEWIGVIYSGNGDTSYAQKFQGRATLTADTSTSTAYMELSSLRSEDTAVYYCARERDTRFGNWGQGTLVTVSS

SEQ ID NO: 56 - protein, 447 aa, synthetic construct
QVQLVQSGAEVKKPGASVKVSCKASGYTFTSYNMHWVRQAPGQGLEWMGVIYSGNGDTSYAQKFQGRVTMTADKSTSTAYMELSSLRSEDTAVYYCARERDTRFGNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 117 aa, synthetic construct
QVQLVQSGAEVKKPGASVKVSCKASGYTFTSYNMHWVRQAPGQGLEWMGVIYSGNGDTSYAQKFQGRVTMTADKSTSTAYMELSSLRSEDTAVYYCARERDTRFGNWGQGTLVTVSS

SEQ ID NO: 58 - protein, 447 aa, synthetic construct
QVQLVQSGAEVKKPGASVKMSCKASGYTFTSYNMHWVRQAPGQGLEWIGVIYSGNGDTSYAQKFQGRATLTADKSTSTAYMELSSLRSEDTAVYYCARERDTRFGNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 117 aa, synthetic construct
QVQLVQSGAEVKKPGASVKMSCKASGYTFTSYNMHWVRQAPGQGLEWIGVIYSGNGDTSYAQKFQGRATLTADKSTSTAYMELSSLRSEDTAVYYCARERDTRFGNWGQGTLVTVSS

SEQ ID NO: 60 - protein, 5 aa, synthetic construct
SYTMH

SEQ ID NO: 61 - protein, 5 aa, synthetic construct
SYQMH

SEQ ID NO: 62 - protein, 5 aa, synthetic construct
NYNMH

SEQ ID NO: 63 - protein, 17 aa, synthetic construct
VIYSGSGDTSYAQKFQG

SEQ ID NO: 64 - protein, 17 aa, synthetic construct
VIYSGQGDTSYAQKFQG

SEQ ID NO: 65 - protein, 17 aa, synthetic construct
VIYSGNGSTSYAQKFQG

SEQ ID NO: 66 - protein, 17 aa, synthetic construct
VIYSGNGETSYAQKFQG

SEQ ID NO: 67 - protein, 17 aa, synthetic construct
VIFSGNGDTSYAQKFQG

SEQ ID NO: 68 - protein, 17 aa, synthetic construct
VIHSGNGDTSYAQKFQG

SEQ ID NO: 69 - protein, 8 aa, synthetic construct
ERETRFGN

SEQ ID NO: 70 - protein, 8 aa, synthetic construct
ERSTRFGN

SEQ ID NO: 71 - protein, 8 aa, synthetic construct
EKDTRFGN

SEQ ID NO: 72 - protein, 8 aa, synthetic construct
ERDTKFGN

SEQ ID NO: 73 - protein, 117 aa, synthetic construct
QVQLVQSGAEVKKPGASVKVSCKASGYTFTSYTMHWVRQAPGQGLEWMGVIYSGNGDTSYAQKFQGRVTMTADTSTSTAYMELSSLRSEDTAVYYCARERDTRFGNWGQGTLVTVSS

SEQ ID NO: 74 - protein, 117 aa, synthetic construct
QVQLVQSGAEVKKPGASVKVSCKASGYTFTSYQMHWVRQAPGQGLEWMGVIYSGNGDTSYAQKFQGRVTMTADTSTSTAYMELSSLRSEDTAVYYCARERDTRFGNWGQGTLVTVSS

SEQ ID NO: 75 - protein, 117 aa, synthetic construct
QVQLVQSGAEVKKPGASVKVSCKASGYTFTNYNMHWVRQAPGQGLEWMGVIYSGNGDTSYAQKFQGRVTMTADTSTSTAYMELSSLRSEDTAVYYCARERDTRFGNWGQGTLVTVSS

SEQ ID NO: 76 - protein, 117 aa, synthetic construct
QVQLVQSGAEVKKPGASVKVSCKASGYTFTSYNMHWVRQAPGQGLEWMGVIYSGSGDTSYAQKFQGRVTMTADTSTSTAYMELSSLRSEDTAVYYCARERDTRFGNWGQGTLVTVSS

SEQ ID NO: 77 - protein, 117 aa, synthetic construct
QVQLVQSGAEVKKPGASVKVSCKASGYTFTSYNMHWVRQAPGQGLEWMGVIYSGQGDTSYAQKFQGRVTMTADTSTSTAYMELSSLRSEDTAVYYCARERDTRFGNWGQGTLVTVSS

SEQ ID NO: 78 - protein, 117 aa, synthetic construct
QVQLVQSGAEVKKPGASVKVSCKASGYTFTSYNMHWVRQAPGQGLEWMGVIYSGNGSTSYAQKFQGRVTMTADTSTSTAYMELSSLRSEDTAVYYCARERDTRFGNWGQGTLVTVSS

SEQ ID NO: 79 - protein, 117 aa, synthetic construct
QVQLVQSGAEVKKPGASVKVSCKASGYTFTSYNMHWVRQAPGQGLEWMGVIYSGNGETSYAQKFQGRVTMTADTSTSTAYMELSSLRSEDTAVYYCARERDTRFGNWGQGTLVTVSS

SEQ ID NO: 80 - protein, 117 aa, synthetic construct
QVQLVQSGAEVKKPGASVKVSCKASGYTFTSYNMHWVRQAPGQGLEWMGVIFSGNGDTSYAQKFQGRVTMTADTSTSTAYMELSSLRSEDTAVYYCARERDTRFGNWGQGTLVTVSS

SEQ ID NO: 81 - protein, 117 aa, synthetic construct
QVQLVQSGAEVKKPGASVKVSCKASGYTFTSYNMHWVRQAPGQGLEWMGVIHSGNGDTSYAQKFQGRVTMTADTSTSTAYMELSSLRSEDTAVYYCARERDTRFGNWGQGTLVTVSS

SEQ ID NO: 82 - protein, 117 aa, synthetic construct
QVQLVQSGAEVKKPGASVKVSCKASGYTFTSYNMHWVRQAPGQGLEWMGVIYSGNGDTSYAQKFQGRVTMTADTSTSTAYMELSSLRSEDTAVYYCARERETRFGNWGQGTLVTVSS

SEQ ID NO: 83 - protein, 117 aa, synthetic construct
QVQLVQSGAEVKKPGASVKVSCKASGYTFTSYNMHWVRQAPGQGLEWMGVIYSGNGDTSYAQKFQGRVTMTADTSTSTAYMELSSLRSEDTAVYYCARERSTRFGNWGQGTLVTVSS

SEQ ID NO: 84 - protein, 117 aa, synthetic construct
QVQLVQSGAEVKKPGASVKVSCKASGYTFTSYNMHWVRQAPGQGLEWMGVIYSGNGDTSYAQKFQGRVTMTADTSTSTAYMELSSLRSEDTAVYYCAREKDTRFGNWGQGTLVTVSS

SEQ ID NO: 85 - protein, 117 aa, synthetic construct
QVQLVQSGAEVKKPGASVKVSCKASGYTFTSYNMHWVRQAPGQGLEWMGVIYSGNGDTSYAQKFQGRVTMTADTSTSTAYMELSSLRSEDTAVYYCARERDTKFGNWGQGTLVTVSS

SEQ ID NO: 86 - protein, 218 aa, synthetic construct
DIQLTQSPSSLSASVGDRATITCRASESVDIYGNSFMHWYQQKPGKAPKLLIYLASNLESGVPSRFSGSGSGTDFTLTISSVQPEDFATYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 111 aa, synthetic construct
DIQLTQSPSSLSASVGDRATITCRASESVDIYGNSFMHWYQQKPGKAPKLLIYLASNLESGVPSRFSGSGSGTDFTLTISSVQPEDFATYYCQQNNEDPYTFGQGTKLEIK

SEQ ID NO: 88 - protein, 218 aa, synthetic construct
DIQLTQSPSSLSASVGDRATITCRASESVDIYGNSFMHWYQQKPGKAPKLLIYLASNLESGVPSRFSGSGSRTDFTLTISSVQPEDFATYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 111 aa, synthetic construct
DIQLTQSPSSLSASVGDRATITCRASESVDIYGNSFMHWYQQKPGKAPKLLIYLASNLESGVPSRFSGSGSRTDFTLTISSVQPEDFATYYCQQNNEDPYTFGQGTKLEIK

SEQ ID NO: 90 - protein, 15 aa, synthetic construct
RASESVDSYGNSFMH

SEQ ID NO: 91 - protein, 15 aa, synthetic construct
RASESVDNYGNSFMH

SEQ ID NO: 92 - protein, 15 aa, synthetic construct
RASESVDIFGNSFMH

SEQ ID NO: 93 - protein, 15 aa, synthetic construct
RASESVDIHGNSFMH

SEQ ID NO: 94 - protein, 15 aa, synthetic construct
RASESVDIYGSSFMH

SEQ ID NO: 95 - protein, 15 aa, synthetic construct
RASESVDIYGQSFMH

SEQ ID NO: 96 - protein, 7 aa, synthetic construct
AASNLES

SEQ ID NO: 97 - protein, 7 aa, synthetic construct
LASSLES

SEQ ID NO: 98 - protein, 9 aa, synthetic construct
QQNQEDPYT

SEQ ID NO: 99 - protein, 9 aa, synthetic construct
QQNSEDPYT

SEQ ID NO: 100 - protein, 9 aa, synthetic construct
QQNNDDPYT

SEQ ID NO: 101 - protein, 9 aa, synthetic construct
QQNNSDPYT

SEQ ID NO: 102 - protein, 9 aa, synthetic construct
QQNNEEPYT

SEQ ID NO: 103 - protein, 9 aa, synthetic construct
QQNNESPYT

SEQ ID NO: 104 - protein, 9 aa, synthetic construct
QQNNEDPFT

SEQ ID NO: 105 - protein, 111 aa, synthetic construct
DIQLTQSPSSLSASVGDRATITCRASESVDSYGNSFMHWYQQKPGKAPKLLIYLASNLESGVPSRFSGSGSGTDFTLTISSVQPEDFATYYCQQNNEDPYTFGQGTKLEIK

SEQ ID NO: 106 - protein, 111 aa, synthetic construct
DIQLTQSPSSLSASVGDRATITCRASESVDNYGNSFMHWYQQKPGKAPKLLIYLASNLESGVPSRFSGSGSGTDFTLTISSVQPEDFATYYCQQNNEDPYTFGQGTKLEIK

SEQ ID NO: 107 - protein, 111 aa, synthetic construct
DIQLTQSPSSLSASVGDRATITCRASESVDIFGNSFMHWYQQKPGKAPKLLIYLASNLESGVPSRFSGSGSGTDFTLTISSVQPEDFATYYCQQNNEDPYTFGQGTKLEIK

SEQ ID NO: 108 - protein, 111 aa, synthetic construct
DIQLTQSPSSLSASVGDRATITCRASESVDIHGNSFMHWYQQKPGKAPKLLIYLASNLESGVPSRFSGSGSGTDFTLTISSVQPEDFATYYCQQNNEDPYTFGQGTKLEIK

SEQ ID NO: 109 - protein, 111 aa, synthetic construct
DIQLTQSPSSLSASVGDRATITCRASESVDIYGSSFMHWYQQKPGKAPKLLIYLASNLESGVPSRFSGSGSGTDFTLTISSVQPEDFATYYCQQNNEDPYTFGQGTKLEIK

SEQ ID NO: 110 - protein, 111 aa, synthetic construct
DIQLTQSPSSLSASVGDRATITCRASESVDIYGQSFMHWYQQKPGKAPKLLIYLASNLESGVPSRFSGSGSGTDFTLTISSVQPEDFATYYCQQNNEDPYTFGQGTKLEIK

SEQ ID NO: 111 - protein, 111 aa, synthetic construct
DIQLTQSPSSLSASVGDRATITCRASESVDIYGNSFMHWYQQKPGKAPKLLIYAASNLESGVPSRFSGSGSGTDFTLTISSVQPEDFATYYCQQNNEDPYTFGQGTKLEIK

SEQ ID NO: 112 - protein, 111 aa, synthetic construct
DIQLTQSPSSLSASVGDRATITCRASESVDIYGNSFMHWYQQKPGKAPKLLIYLASSLESGVPSRFSGSGSGTDFTLTISSVQPEDFATYYCQQNNEDPYTFGQGTKLEIK

SEQ ID NO: 113 - protein, 111 aa, synthetic construct
DIQLTQSPSSLSASVGDRATITCRASESVDIYGNSFMHWYQQKPGKAPKLLIYLASNLESGVPSRFSGSGSGTDFTLTISSVQPEDFATYYCQQNQEDPYTFGQGTKLEIK

SEQ ID NO: 114 - protein, 111 aa, synthetic construct
DIQLTQSPSSLSASVGDRATITCRASESVDIYGNSFMHWYQQKPGKAPKLLIYLASNLESGVPSRFSGSGSGTDFTLTISSVQPEDFATYYCQQNSEDPYTFGQGTKLEIK

SEQ ID NO: 115 - protein, 111 aa, synthetic construct
DIQLTQSPSSLSASVGDRATITCRASESVDIYGNSFMHWYQQKPGKAPKLLIYLASNLESGVPSRFSGSGSGTDFTLTISSVQPEDFATYYCQQNNDDPYTFGQGTKLEIK

SEQ ID NO: 116 - protein, 111 aa, synthetic construct
DIQLTQSPSSLSASVGDRATITCRASESVDIYGNSFMHWYQQKPGKAPKLLIYLASNLESGVPSRFSGSGSGTDFTLTISSVQPEDFATYYCQQNNSDPYTFGQGTKLEIK

SEQ ID NO: 117 - protein, 111 aa, synthetic construct
DIQLTQSPSSLSASVGDRATITCRASESVDIYGNSFMHWYQQKPGKAPKLLIYLASNLESGVPSRFSGSGSGTDFTLTISSVQPEDFATYYCQQNNEEPYTFGQGTKLEIK

SEQ ID NO: 118 - protein, 111 aa, synthetic construct
DIQLTQSPSSLSASVGDRATITCRASESVDIYGNSFMHWYQQKPGKAPKLLIYLASNLESGVPSRFSGSGSGTDFTLTISSVQPEDFATYYCQQNNESPYTFGQGTKLEIK

SEQ ID NO: 119 - protein, 111 aa, synthetic construct
DIQLTQSPSSLSASVGDRATITCRASESVDIYGNSFMHWYQQKPGKAPKLLIYLASNLESGVPSRFSGSGSGTDFTLTISSVQPEDFATYYCQQNNEDPFTFGQGTKLEIK

SEQ ID NO: 120 - protein, 445 aa, synthetic construct
KVQLLESGGGLVQPGRSLRLSCAASGFTFDDYGMHWVRQAPGKGLEWVSGISWNSGSIGYADSVKGRFTISRDNSKNTLYLQMNSLRAEDTAVYYCARGGQTIDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 121 - protein, 116 aa, synthetic construct
KVQLLESGGGLVQPGRSLRLSCAASGFTFDDYGMHWVRQAPGKGLEWVSGISWNSGSIGYADSVKGRFTISRDNSKNTLYLQMNSLRAEDTAVYYCARGGQTIDIWGQGTMVTVSS

SEQ ID NO: 122 - protein, 5 aa, synthetic construct
DYGMH

SEQ ID NO: 123 - protein, 17 aa, synthetic construct
GISWNSGSIGYADSVKG

SEQ ID NO: 124 - protein, 7 aa, synthetic construct
GGQTIDI

SEQ ID NO: 125 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIK

SEQ ID NO: 127 - protein, 11 aa, synthetic construct
RASQSISSYLN

SEQ ID NO: 128 - protein, 7 aa, synthetic construct
AASSLQS

SEQ ID NO: 129 - protein, 9 aa, synthetic construct
QQSYSTPFT

SEQ ID NO: 130 - protein, 330 aa, Homo sapiens [sequence omitted]

SEQ ID NO: 131 - protein, 107 aa, Homo sapiens [sequence omitted]

SEQ ID NO: 132 - protein, 114 aa, synthetic construct
EVQLQQSVAELVRPGASVKLSCTGSGFNIENTYTHWVKQRPEQGLEWIGRIDPANTNTKYAPKFQGKATITADTSSNTAFLQLSSLTSEDTAIYYCALYDEDFWGPGTTLTVSS

SEQ ID NO: 133 - protein, 5 aa, synthetic construct
NTYTH

SEQ ID NO: 134 - protein, 17 aa, synthetic construct
RIDPANTNTKYAPKFQG

SEQ ID NO: 135 - protein, 5 aa, synthetic construct
YDEDF

SEQ ID NO: 136 - protein, 106 aa, synthetic construct
QIVLTQSPAIMSASPGEKVTMTCSASSSLTYMHWYQQKSGTSPKRWIYDTSELASGVPARFSGSGSGTSYSLTISSMEAEDAATYYCQQWNSDPPTFGGGTKLEIK

SEQ ID NO: 137 - protein, 10 aa, synthetic construct
SASSSLTYMH

SEQ ID NO: 138 - protein, 7 aa, synthetic construct
DTSELAS

SEQ ID NO: 139 - protein, 9 aa, synthetic construct
QQWNSDPPT

SEQ ID NO: 140 - protein, 114 aa, synthetic construct
EFQLQQSGPELVKPGASVKISCKASDYSFTDYNMNWVKQSNGKSLEWIGVINPNYGTTSYNQKFKGKATLTVDQSSSTAYMQLNSLTSEDSAVYYCARSGGFSWGQGTLVTVSA

SEQ ID NO: 141 - protein, 5 aa, synthetic construct
DYNMN

SEQ ID NO: 142 - protein, 17 aa, synthetic construct
VINPNYGTTSYNQKFKG

SEQ ID NO: 143 - protein, 5 aa, synthetic construct
SGGFS

SEQ ID NO: 144 - protein, 108 aa, synthetic construct
DIVMTQSHTFMSTSVGDRVSITCKASQDVSTAVAWYQQKPGQSPKLLIFSASYRYTGVPDRFTGSGSGTDFTFTISSVQAEDLAVYYCQQHYSTPTWTFGGGTKLEIK

SEQ ID NO: 145 - protein, 11 aa, synthetic construct
KASQDVSTAVA

SEQ ID NO: 146 - protein, 7 aa, synthetic construct
SASYRYT

SEQ ID NO: 147 - protein, 10 aa, synthetic construct
QQHYSTPTWT

SEQ ID NO: 148 - protein, 116 aa, synthetic construct
EVQLVESGGGLVQPKGSLKLSCAASGFTFKNYVMHWVRQTPGKGLEWVARIRSKNSNYATYYADSVKDRFTISRDDSQSMLYLQMNNLKTEDTAMYYCMRALVAYWGQGTLVTVSA

SEQ ID NO: 149 - protein, 5 aa, synthetic construct
NYVMH

SEQ ID NO: 150 - protein, 19 aa, synthetic construct
RIRSKNSNYATYYADSVKD

SEQ ID NO: 151 - protein, 5 aa, synthetic construct
ALVAY

SEQ ID NO: 152 - protein, 106 aa, synthetic construct
QLVLTQSPAIMSTSPGEKVTMTCSASSSVSYIYWYQQKPGSSPRLLIYDTSNLASGVPVRFSGSGSGTSYSLTISRMEAEDAATYYCQQWSSYPYTFGGGTKLEIK

SEQ ID NO: 153 - protein, 10 aa, synthetic construct
SASSSVSYIY

SEQ ID NO: 154 - protein, 7 aa, synthetic construct
DTSNLAS

SEQ ID NO: 155 - protein, 9 aa, synthetic construct
QQWSSYPYT

SEQ ID NO: 156 - protein, 446 aa, synthetic construct
EVQLVESGGGLVQPKGSLKLSCAASGFTFKNYVMHWVRQTPGKGLEWVARIRSKNSNYATYYADSVKDRFTISRDDSQSMLYLQMNNLKTEDTAMYYCMRALVAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 213 aa, synthetic construct
QLVLTQSPAIMSTSPGEKVTMTCSASSSVSYIYWYQQKPGSSPRLLIYDTSNLASGVPVRFSGSGSGTSYSLTISRMEAEDAATYYCQQWSSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - protein, 446 aa, synthetic construct
EVQLVESGGGLVQPGGSLRLSCAASGFTFKNYVMHWVRQAPGKGLEWVSRIRSKNSNYATYYADSVKGRFTISRDNSKNTLYLQMNSLRAEDTAVYYCAKALVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 116 aa, synthetic construct
EVQLVESGGGLVQPGGSLRLSCAASGFTFKNYVMHWVRQAPGKGLEWVSRIRSKNSNYATYYADSVKGRFTISRDNSKNTLYLQMNSLRAEDTAVYYCAKALVAYWGQGTLVTVSS

SEQ ID NO: 160 - protein, 19 aa, synthetic construct
RIRSKNSNYATYYADSVKG

SEQ ID NO: 161 - protein, 446 aa, synthetic construct
EVQLVESGGGLVQPGGSLRLSCAASGFTFKNYVMHWVRQAPGKGLEWVARIRSKNSNYATYYADSVKGRFTISRDDSKNTLYLQMNSLRAEDTAVYYCMRALVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 116 aa, synthetic construct
EVQLVESGGGLVQPGGSLRLSCAASGFTFKNYVMHWVRQAPGKGLEWVARIRSKNSNYATYYADSVKGRFTISRDDSKNTLYLQMNSLRAEDTAVYYCMRALVAYWGQGTLVTVSS

SEQ ID NO: 163 - protein, 213 aa, synthetic construct
DIQMTQSPSSLSASVGDRVTITCRASSSVSYIYWYQQKPGKAPKLLIYDTSNLASGVPSRFSGSGSGTDFTLTISSLQPEDFATYYCQQWSSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 106 aa, synthetic construct
DIQMTQSPSSLSASVGDRVTITCRASSSVSYIYWYQQKPGKAPKLLIYDTSNLASGVPSRFSGSGSGTDFTLTISSLQPEDFATYYCQQWSSYPYTFGQGTKLEIK

SEQ ID NO: 165 - protein, 10 aa, synthetic construct
RASSSVSYIY

SEQ ID NO: 166 - protein, 213 aa, synthetic construct
DIQMTQSPSSLSASVGDRVTITCRASSSVSYIYWYQQKPGKAPKLLIYDTSNLASGVPSRFSGSGSGTDYTLTISSLQPEDFATYYCQQWSSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 167 - protein, 106 aa, synthetic construct
DIQMTQSPSSLSASVGDRVTITCRASSSVSYIYWYQQKPGKAPKLLIYDTSNLASGVPSRFSGSGSGTDYTLTISSLQPEDFATYYCQQWSSYPYTFGQGTKLEIK

SEQ ID NO: 168 - protein, 213 aa, synthetic construct
DIQLTQSPSSLSASVGDRVTMTCRASSSVSYIYWYQQKPGKAPKLLIYDTSNLASGVPSRFSGSGSGTDYTLTISSMQPEDFATYYCQQWSSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 169 - protein, 106 aa, synthetic construct
DIQLTQSPSSLSASVGDRVTMTCRASSSVSYIYWYQQKPGKAPKLLIYDTSNLASGVPSRFSGSGSGTDYTLTISSMQPEDFATYYCQQWSSYPYTFGQGTKLEIK

(19) United States
(12) Patent Application Publication
(10) Pub. No.: US 2026/0070975 A1
(43) Pub. Date: Mar. 12, 2026

(54) ANTIBODIES TARGETING C-KIT AND/OR SIGLEC AND USES THEREOF

(71) Applicant: Santa Ana Bio, Inc., Alameda, CA (US)

(72) Inventors: Peter Emtage, Lafayette, CA (US); Eugene Bolotin, Berkeley, CA (US); Nancy Emtage, Alameda, CA (US)

(21) Appl. No.: 18/868,046

(22) PCT Filed: Jun. 9, 2023
(86) PCT No.: PCT/US2023/068248

§ 371 Date: Nov. 21, 2024

Related U.S. Application Data
(60) Provisional application No. 63/350,832, filed on Jun. 9, 2022.
(60) Provisional application No. 63/481,257, filed on Jan. 24, 2023.

(51) Int. Cl. C07K 16/28; A61K 47/68; A61P 35/00; C07K 16/24

(52) U.S. Cl. CPC C07K 16/2803; A61K 47/6879; A61P 35/00; C07K 16/24; C07K 2317/14; C07K 2317/24; C07K 2317/31; C07K 2317/41; C07K 2317/51; C07K 2317/515; C07K 2317/522; C07K 2317/524; C07K 2317/565; C07K 2317/71; C07K 2317/72; C07K 2317/73; C07K 2317/77; C07K 2317/92

(57) ABSTRACT

Undesired mast cell activities are linked with various diseases or disorders, such as allergies, inflammatory diseases, autoimmune diseases, and cancers. There remains a need for new and improved therapeutics (e.g., for mast cell associated diseases or disorders) that can target c-Kit and/or siglec-6. Disclosed herein are molecules, such as antibodies and antibody fragments, capable of binding to c-Kit and/or siglec-6.

### RELATED APPLICATIONS

The present application claims priority to U.S. Provisional Patent Application No. 63/350,832, filed Jun. 9, 2022; and U.S. Provisional Patent Application No. 63/481,257, filed Jan. 24, 2023, the entire contents of each which are hereby incorporated by reference for all purposes.

### BACKGROUND

Mast cells (MCs) are innate immune cells that play a key role in the inflammatory process. When activated, MCs secrete various immune mediators such as cytokines, leukotrienes, and a large number of proteases into the environment. Mast cell differentiation, growth, and survival are strongly regulated by local tissue environmental factors. Stem cell factor (SCF) and IL-3 are among the best-characterized factors. Undesired mast cell activities have been linked with various diseases or disorders, such as allergies, inflammatory diseases, autoimmune diseases, and cancers.

c-Kit is a type-III receptor tyrosine kinase and a well-known cell surface receptor that binds to its physiological ligand, SCF. c-Kit is involved in various intracellular signaling pathways and have multiple functions during embryogenesis and adulthood. c-Kit is highly expressed in MCs; upon complete differentiation, MCs rely on c-Kit-dependent signaling for survival, function, and growth. c-Kit antibodies, such as CDX-0158, have been developed.

Siglec-6 is a member of the CD33-related subfamily of sialic acid-binding immunoglobulin-like lectins (siglecs). Siglec-6 is found on human mast cells, some B cells, and cyto- and syncytiotrophoblasts of the placenta. Siglec-6 has been suggested to possess inhibitory activity for both IgE- and non-IgE-mediated mast cell responses.

There remains a need for new and improved therapeutics (e.g., for mast cell associated diseases or disorders) that can target c-Kit and/or siglec-6.

### SUMMARY

The present application provides antigen-binding sites that are capable of binding c-Kit and/or siglec-6.

In one aspects, provided are antibodies or antigen-binding fragments thereof, comprising a first antigen-binding domain that is capable of binding siglec-6 and a second antigen-binding domain that is capable of binding c-Kit.

In some embodiments, the first antigen-binding domain is capable of binding siglec-6 with a higher affinity than the second antigen-binding domain is capable of binding c-Kit. In some embodiments, the first antigen-binding domain is capable of binding siglec-6 with an affinity characterized by a K_{D} value that is lower than about 100 uM, lower than about 100 nM, lower than about 10 nM, lower than about 1 nM, lower than about 100 pM, lower than about 10 pM, lower than about 1 pM, lower than about 100 fM, or lower than about 10 fM. In some embodiments, the first antigen-binding domain is capable of binding c-Kit with an affinity characterized by a K_{D} value that is between about 1 nM and about 900 uM, between about 5 nM and about 500 uM, between about 10 nM and about 100 uM, between about 20 nM and about 10 uM, or between about 25 and about 500 nM.

In some embodiments, the antibody or antigen-binding fragment is capable of binding a cell expressing siglect-6.

In some embodiments, the antibody or antigen-binding fragment is capable of binding a mast cell. In some embodiments, the antibody or antigen-binding fragment binds a mast cell preferentially than another immune cell. In some embodiments, the immune cell is T cell, dendritic cell, macrophage, neutrophil, or basophil.

In some embodiments, the antibody or antigen-binding fragment thereof does not induce mast cell degranulation.

In some embodiments, the antibody or antigen-binding fragment thereof has a comparable or lower internalization rate compared to a reference antibody capable of binding siglect-6. In some embodiments, the reference antibody capable of binding siglect-6 comprises a heavy chain variable (VH) region comprising the amino acid sequence of SEQ ID NO: 2, and a light chain variable (VL) region comprising the amino acid sequence of SEQ ID NO: 10.

In some embodiments, the antibody or antigen-binding fragment thereof has a comparable or lower toxicity compared to a reference antibody capable of binding c-Kit. In some embodiments, the reference antibody capable of binding c-Kit comprises a VH region comprising the amino acid sequence of SEQ ID NO: 40, and a VL region comprising the amino acid sequence of SEQ ID NO: 45.

In some embodiments, the first antigen-binding domain comprises:
(a) a VH region comprising: heavy chain complementarity-determining region 1 (HCDR1) having the amino acid sequence of SEQ ID NO: 3, or a sequence differing in 1 or 2 amino acids therefrom, HCDR2 having the amino acid sequence of SEQ ID NO: 4 or 21, or a sequence differing in 1 or 2 amino acids therefrom, and HCDR3 having the amino acid sequence of SEQ ID NO: 5, or a sequence differing in 1 or 2 amino acids therefrom; and
(b) a VL region comprising: heavy chain complementarity-determining region 1 (LCDR1) having the amino acid sequence of SEQ ID NO: 11 or 32, or a sequence differing in 1 or 2 amino acids therefrom, LCDR2 having the amino acid sequence of SEQ ID NO: 12, or a sequence differing in 1 or 2 amino acids therefrom, and LCDR3 having the amino acid sequence of SEQ ID NO: 13, or a sequence differing in 1 or 2 amino acids therefrom.

In some embodiments, the first antigen-binding domain comprises:
(a) a VH region comprising: HCDR1 having the amino acid sequence of SEQ ID NO: 3, HCDR2 having the amino acid sequence of SEQ ID NO: 4 or 21, and HCDR3 having the amino acid sequence of SEQ ID NO: 5; and
(b) a VL region comprising: LCDR1 having the amino acid sequence of SEQ ID NO: 11 or 32, LCDR2 having the amino acid sequence of SEQ ID NO: 12, and LCDR3 having the amino acid sequence of SEQ ID NO: 13.

In some embodiments, the first antigen-binding domain comprises:
(a) a VH region comprising:
   (i) HCDR1, HCDR2, and HCDR3 having the amino acid sequence of SEQ ID NOs: 3, 4, and 5, respectively; or
   (ii) HCDR1, HCDR2, and HCDR3 having the amino acid sequence of SEQ ID NOs: 3, 21, and 5, respectively; and
(b) a VL region comprising:
   (i) LCDR1, LCDR2, and LCDR3 having the amino acid sequence of SEQ ID NOs: 11, 12, and 13, respectively; or
   (ii) LCDR1, LCDR2, and LCDR3 having the amino acid sequence of SEQ ID NOs: 32, 12, and 13, respectively.

In some embodiments, the first antigen-binding domain comprises:
(a) a VH region comprising an amino acid sequence having at least 70%, at least 80%, at least 90%, or at least 95% sequence identity to the amino acid sequence of SEQ ID NO: 2, 20, 23, 25, 27, or 29; and
(b) a VL region comprising an amino acid sequence having at least 70%, at least 80%, at least 90%, or at least 95% sequence identity to the amino acid sequence of SEQ ID NO: 10, 31, 36, or 38.

In some embodiments, the first antigen-binding domain comprises:
(a) a VH region having the amino acid sequence of SEQ ID NO: 2, 20, 23, 25, 27, or 29; and
(b) a VL region having the amino acid sequence of SEQ ID NO: 10, 31, 36, or 38.

In some embodiments, the first antigen-binding domain comprises:
(a) a VH region having the amino acid sequence of SEQ ID NO: 2, and a VL region having the amino acid sequence of SEQ ID NO: 10;
(b) a VH region having the amino acid sequence of SEQ ID NO: 23, and a VL region having the amino acid sequence of SEQ ID NO: 38;
(c) a VH region having the amino acid sequence of SEQ ID NO: 25, and a VL region having the amino acid sequence of SEQ ID NO: 38;
(d) a VH region having the amino acid sequence of SEQ ID NO: 27, and a VL region having the amino acid sequence of SEQ ID NO: 38; or
(e) a VH region having the amino acid sequence of SEQ ID NO: 29, and a VL region having the amino acid sequence of SEQ ID NO: 38.

In some embodiments, the first antigen-binding domain comprises: a VH region having the amino acid sequence of SEQ ID NO: 25, and a VL region having the amino acid sequence of SEQ ID NO: 38.

In some embodiments, the first antigen-binding domain comprises:
(a) a VH region comprising:
   HCDR1 having the amino acid sequence of SEQ ID NO: 122, or a sequence differing in 1 or 2 amino acids therefrom,
   HCDR2 having the amino acid sequence of SEQ ID NO: 123, or a sequence differing in 1 or 2 amino acids therefrom, and
   HCDR3 having the amino acid sequence of SEQ ID NO: 124, or a sequence differing in 1 or 2 amino acids therefrom; and
(b) a VL region comprising:
   LCDR1 having the amino acid sequence of SEQ ID NO: 127, or a sequence differing in 1 or 2 amino acids therefrom,
   LCDR2 having the amino acid sequence of SEQ ID NO: 128, or a sequence differing in 1 or 2 amino acids therefrom, and
   LCDR3 having the amino acid sequence of SEQ ID NO: 129, or a sequence differing in 1 or 2 amino acids therefrom.

In some embodiments, the first antigen-binding domain comprises:
(a) a VH region comprising:
   HCDR1 having the amino acid sequence of SEQ ID NO: 122,
   HCDR2 having the amino acid sequence of SEQ ID NO: 123, and
   HCDR3 having the amino acid sequence of SEQ ID NO: 124; and
(b) a VL region comprising:
   LCDR1 having the amino acid sequence of SEQ ID NO: 127,
   LCDR2 having the amino acid sequence of SEQ ID NO: 128, and
   LCDR3 having the amino acid sequence of SEQ ID NO: 139.

In some embodiments, the first antigen-binding domain comprises:
(a) a VH region comprising an amino acid sequence having at least 70%, at least 80%, at least 90%, or at least 95% sequence identity to the amino acid sequence of SEQ ID NO: 121; and
(b) a VL region comprising an amino acid sequence having at least 70%, at least 80%, at least 90%, or at least 95% sequence identity to the amino acid sequence of SEQ ID NO: 126.

In some embodiments, the first antigen-binding domain comprises:
(a) a VH region having the amino acid sequence of SEQ ID NO: 121; and
(b) a VL region having the amino acid sequence of SEQ ID NO: 126.

In some embodiments, the first antigen-binding domain comprises:
(a) a VH region comprising:
   HCDR1 having the amino acid sequence of SEQ ID NO: 133, 141, or 149, or a sequence differing in 1 or 2 amino acids therefrom,
   HCDR2 having the amino acid sequence of SEQ ID NO: 134, 142, 150, or 160, or a sequence differing in 1 or 2 amino acids therefrom, and
   HCDR3 having the amino acid sequence of SEQ ID NO: 135, 143, or 151, or a sequence differing in 1 or 2 amino acids therefrom; and
(b) a VL region comprising:
   LCDR1 having the amino acid sequence of SEQ ID NO: 137, 145, 153, or 165, or a sequence differing in 1 or 2 amino acids therefrom,
   LCDR2 having the amino acid sequence of SEQ ID NO: 138, 146, or 154, or a sequence differing in 1 or 2 amino acids therefrom, and
   LCDR3 having the amino acid sequence of SEQ ID NO: 139, 147, or 155, or a sequence differing in 1 or 2 amino acids therefrom.

In some embodiments, the first antigen-binding domain comprises:
(a) a VH region comprising:
   HCDR1 having the amino acid sequence of SEQ ID NO: 133, 141, or 149,
   HCDR2 having the amino acid sequence of SEQ ID NO: 134, 142, 150, or 160, and
   HCDR3 having the amino acid sequence of SEQ ID NO: 135, 143, or 151; and
(b) a VL region comprising:
   LCDR1 having the amino acid sequence of SEQ ID NO: 137, 145, 153, or 165,
   LCDR2 having the amino acid sequence of SEQ ID NO: 138, 146, or 154, and
   LCDR3 having the amino acid sequence of SEQ ID NO: 139, 147, or 155.

In some embodiments, the first antigen-binding domain comprises:
(a) a VH region comprising:
   (i) HCDR1, HCDR2, and HCDR3 having the amino acid sequence of SEQ ID NOs: 133, 134, and 135, respectively;
   (ii) HCDR1, HCDR2, and HCDR3 having the amino acid sequence of SEQ ID NOs: 141, 142, and 143, respectively;
   (iii) HCDR1, HCDR2, and HCDR3 having the amino acid sequence of SEQ ID NOs: 149, 150, and 151, respectively; or
   (iv) HCDR1, HCDR2, and HCDR3 having the amino acid sequence of SEQ ID NOs: 149, 160, and 151, respectively; and
(b) a VL region comprising:
   (i) LCDR1, LCDR2, and LCDR3 having the amino acid sequence of SEQ ID NOs: 137, 138, and 139, respectively;
   (ii) LCDR1, LCDR2, and LCDR3 having the amino acid sequence of SEQ ID NOs: 145, 146, and 147, respectively;
   (iii) LCDR1, LCDR2, and LCDR3 having the amino acid sequence of SEQ ID NOs: 153, 154, and 155, respectively; or
   (iv) LCDR1, LCDR2, and LCDR3 having the amino acid sequence of SEQ ID NOs: 165, 154, and 155, respectively.

In some embodiments, the first antigen-binding domain comprises:
(a) a VH region comprising an amino acid sequence having at least 70%, at least 80%, at least 90%, or at least 95% sequence identity to the amino acid sequence of SEQ ID NO: 132, 140, 148, 159, or 162; and
(b) a VL region comprising an amino acid sequence having at least 70%, at least 80%, at least 90%, or at least 95% sequence identity to the amino acid sequence of SEQ ID NO: 136, 144, 152, 164, 167, or 169.

In some embodiments, the first antigen-binding domain comprises:
(a) a VH region having the amino acid sequence of SEQ ID NO: 132, 140, 148, 159, or 162; and
(b) a VL region having the amino acid sequence of SEQ ID NO: 136, 144, 152, 164, 167, or 169.

In some embodiments, the first antigen-binding domain comprises:
(a) a VH region having the amino acid sequence of SEQ ID NO: 132, and a VL region having the amino acid sequence of SEQ ID NO: 139;
(b) a VH region having the amino acid sequence of SEQ ID NO: 140, and a VL region having the amino acid sequence of SEQ ID NO: 144; or
(c) a VH region having the amino acid sequence of SEQ ID NO: 148, and a VL region having the amino acid sequence of SEQ ID NO: 152.

In some embodiments, the first antigen-binding domain comprises:
(a) a VH region having the amino acid sequence of SEQ ID NO: 159, and a VL region having the amino acid sequence of SEQ ID NO: 164;
(6) a VH region having the amino acid sequence of SEQ ID NO: 162, and a VL region having the amino acid sequence of SEQ ID NO: 167; or
(c) a VH region having the amino acid sequence of SEQ ID NO: 162, and a VL region having the amino acid sequence of SEQ ID NO: 169.

In some embodiments, the second antigen-binding domain comprises:
(a) a VH region comprising:
   HCDR1 having the amino acid sequence of SEQ ID NO: 41, or a sequence differing in 1 or 2 amino acids therefrom,
   HCDR2 having the amino acid sequence of SEQ ID NO: 42 or 53, or a sequence differing in 1 or 2 amino acids therefrom, and
   HCDR3 having the amino acid sequence of SEQ ID NO: 43, or a sequence differing in 1 or 2 amino acids therefrom; and
(b) a VL region comprising:
   LCDR1 having the amino acid sequence of SEQ ID NO: 46, or a sequence differing in 1 or 2 amino acids therefrom,
   LCDR2 having the amino acid sequence of SEQ ID NO: 47, or a sequence differing in 1 or 2 amino acids therefrom, and
   LCDR3 having the amino acid sequence of SEQ ID NO: 48, or a sequence differing in 1 or 2 amino acids therefrom.

In some embodiments, the second antigen-binding domain comprises:
(a) a VH region comprising:
   HCDR1 having the amino acid sequence of SEQ ID NO: 41,
   HCDR2 having the amino acid sequence of SEQ ID NO: 42 or 53, and
   HCDR3 having the amino acid sequence of SEQ ID NO: 43; and
(b) a VL region comprising:
   LCDR1 having the amino acid sequence of SEQ ID NO: 46,
   LCDR2 having the amino acid sequence of SEQ ID NO: 47, and
   LCDR3 having the amino acid sequence of SEQ ID NO: 48.

In some embodiments, the second antigen-binding domain comprises a VH region comprising:
(a) HCDR1 having the amino acid sequence of SEQ ID NO: 60, 61, or 62,
(b) HCDR2 having the amino acid sequence of SEQ ID NO: 63, 64, 65, 66, 67, or 68, and
(c) HCDR3 having the amino acid sequence of SEQ ID NO: 69, 70, 71, or 72.

In some embodiments, the second antigen-binding domain comprises a VH region comprising:
(a) LCDR1 having the amino acid sequence of SEQ ID NO: 90, 91, 92, 93, 94, or 95,
(b) LCDR2 having the amino acid sequence of SEQ ID NO: 96 or 97, and
(c) LCDR3 having the amino acid sequence of SEQ ID NO: 98, 99, 100, 101, 102, 103, or 104.

In some embodiments, the second antigen-binding domain comprises:
(a) a VH region comprising:
   (i) HCDR1, HCDR2, and HCDR3 having the amino acid sequence of SEQ ID NOs: 41, 42, and 43, respectively;
   (ii) HCDR1, HCDR2, and HCDR3 having the amino acid sequence of SEQ ID NOs: 41, 53, and 43, respectively;
   (iii) HCDR1, HCDR2, and HCDR3 having the amino acid sequence of SEQ ID NOs: 60, 53, and 43, respectively;
   (iv) HCDR1, HCDR2, and HCDR3 having the amino acid sequence of SEQ ID NOs: 61, 53, and 43, respectively;
   (v) HCDR1, HCDR2, and HCDR3 having the amino acid sequence of SEQ ID NOs: 62, 53, and 43, respectively;
   (vi) HCDR1, HCDR2, and HCDR3 having the amino acid sequence of SEQ ID NOs: 41, 63, and 43, respectively;
   (vii) HCDR1, HCDR2, and HCDR3 having the amino acid sequence of SEQ ID NOs: 41, 64, and 43, respectively;
   (viii) HCDR1, HCDR2, and HCDR3 having the amino acid sequence of SEQ ID NOs: 41, 65, and 43, respectively;
   (ix) HCDR1, HCDR2, and HCDR3 having the amino acid sequence of SEQ ID NOs: 41, 66, and 43, respectively;
   (x) HCDR1, HCDR2, and HCDR3 having the amino acid sequence of SEQ ID NOs: 41, 67, and 43, respectively;
   (xi) HCDR1, HCDR2, and HCDR3 having the amino acid sequence of SEQ ID NOs: 41, 68, and 43, respectively;
   (xii) HCDR1, HCDR2, and HCDR3 having the amino acid sequence of SEQ ID NOs: 41, 53, and 69, respectively;
   (xiii) HCDR1, HCDR2, and HCDR3 having the amino acid sequence of SEQ ID NOs: 41, 53, and 70, respectively;
   (xiv) HCDR1, HCDR2, and HCDR3 having the amino acid sequence of SEQ ID NOs: 41, 53, and 71, respectively; or
   (xv) HCDR1, HCDR2, and HCDR3 having the amino acid sequence of SEQ ID NOs: 41, 53, and 72, respectively; and
(b) a VL region comprising:
   (i) LCDR1, LCDR2, and LCDR3 having the amino acid sequence of SEQ ID NOs: 46, 47, and 48, respectively;
   (ii) LCDR1, LCDR2, and LCDR3 having the amino acid sequence of SEQ ID NOs: 90, 47, and 48, respectively;
   (iii) LCDR1, LCDR2, and LCDR3 having the amino acid sequence of SEQ ID NOs: 91, 47, and 48, respectively;
   (iv) LCDR1, LCDR2, and LCDR3 having the amino acid sequence of SEQ ID NOs: 92, 47, and 48, respectively;
   (v) LCDR1, LCDR2, and LCDR3 having the amino acid sequence of SEQ ID NOs: 93, 47, and 48, respectively;
   (vi) LCDR1, LCDR2, and LCDR3 having the amino acid sequence of SEQ ID NOs: 94, 47, and 48, respectively;
   (vii) LCDR1, LCDR2, and LCDR3 having the amino acid sequence of SEQ ID NOs: 95, 47, and 48, respectively;
   (viii) LCDR1, LCDR2, and LCDR3 having the amino acid sequence of SEQ ID NOs: 46, 96, and 48, respectively;
   (ix) LCDR1, LCDR2, and LCDR3 having the amino acid sequence of SEQ ID NOs: 46, 97, and 48, respectively;
   (x) LCDR1, LCDR2, and LCDR3 having the amino acid sequence of SEQ ID NOs: 46, 47, and 98, respectively;
   (xi) LCDR1, LCDR2, and LCDR3 having the amino acid sequence of SEQ ID NOs: 46, 47, and 99, respectively;
   (xii) LCDR1, LCDR2, and LCDR3 having the amino acid sequence of SEQ ID NOs: 46, 47, and 100, respectively;
   (xiii) LCDR1, LCDR2, and LCDR3 having the amino acid sequence of SEQ ID NOs: 46, 47, and 101, respectively;
   (xiv) LCDR1, LCDR2, and LCDR3 having the amino acid sequence of SEQ ID NOs: 46, 47, and 102, respectively;
   (xv) LCDR1, LCDR2, and LCDR3 having the amino acid sequence of SEQ ID NOs: 46, 47, and 103, respectively; or
   (xvi) LCDR1, LCDR2, and LCDR3 having the amino acid sequence of SEQ ID NOs: 46, 47, and 104, respectively.

In some embodiments, the second antigen-binding domain comprises:
(a) a VH region comprising an amino acid sequence having at least 70%, at least 80%, at least 90%, or at least 95% sequence identity to the amino acid sequence of SEQ ID NO: 40, 52, 55, 57, 59, 73, 74, 75, 76, 77, 78, 79, 80, 81, 82, 83, 84, or 85; and
(b) a VL region comprising an amino acid sequence having at least 70%, at least 80%, at least 90%, or at least 95% sequence identity to the amino acid sequence of SEQ ID NO: 45, 87, 89, 105, 106, 107, 108, 109, 110, 111, 112, 113, 114, 115, 116, 117, 118, or 119.

In some embodiments, the second antigen-binding domain comprises:
(a) a VH region having the amino acid sequence of SEQ ID NO: 40, 52, 55, 57, 59, 73, 74, 75, 76, 77, 78, 79, 80, 81, 82, 83, 84, or 85; and
(b) a VL region having the amino acid sequence of SEQ ID NO: 45, 87, 89, 105, 106, 107, 108, 109, 110, 111, 112, 113, 114, 115, 116, 117, 118, or 119.

In some embodiments, the second antigen-binding domain comprises:
(a) a VH region having the amino acid sequence of SEQ ID NO: 40, and a VL region having the amino acid sequence of SEQ ID NO: 45;
(b) a VH region having the amino acid sequence of SEQ ID NO: 52, and a VL region having the amino acid sequence of SEQ ID NO: 87;
(c) a VH region having the amino acid sequence of SEQ ID NO: 55, and a VL region having the amino acid sequence of SEQ ID NO: 87;
(d) a VH region having the amino acid sequence of SEQ ID NO: 57, and a VL region having the amino acid sequence of SEQ ID NO: 87;
(e) a VH region having the amino acid sequence of SEQ ID NO: 59, and a VL region having the amino acid sequence of SEQ ID NO: 87;
(f) a VH region having the amino acid sequence of SEQ ID NO: 52, and a VL region having the amino acid sequence of SEQ ID NO: 89; or
(g) a VH region having the amino acid sequence of SEQ ID NO: 55, and a VL region having the amino acid sequence of SEQ ID NO: 89.

In some embodiments, the second antigen-binding domain comprises: a VH region having the amino acid sequence of SEQ ID NO: 52, and a VL region having the amino acid sequence of SEQ ID NO: 87.

In some embodiments, the antibody or antigen-binding fragment thereof comprises:
(a) a first heavy chain, comprising the VH region of the first antigen-binding domain, and a first heavy chain constant (CH) region or a fragment thereof;
(b) a first light chain, comprising the VL region of the first antigen-binding domain, and a first light chain constant (CL) region or a fragment thereof;
(c) a second heavy chain, comprising the VH region of the second antigen-binding domain, and a second CH region or a fragment thereof, and
(d) a second light chain, comprising the VL region of the second antigen-binding domain, and a second CL region or a fragment thereof.

In some embodiments, the first and second CH region each comprise an amino acid sequence having at least 70%, at least 80%, at least 90%, or at least 95% sequence identity to the amino acid sequence of SEQ ID NO: 130.

In some embodiments, the first and second heavy chain form a heterodimer, optionally wherein one of the first and second heavy chains comprises a Serine residue at position 366, an Alanine residue at position 368 and a Valine residue at position 407; and the other heavy chain comprises a Tryptophan residue at position 366, wherein the numbering of the constant region is as per the EU index.

In some embodiments, the first and/or second CH region comprise one or more mutations that reduce or abrogate effector function. In some embodiments, the first and/or second CH region is a human IgG1 CH comprising one of the following mutation(s), wherein the numbering of the constant region is as per the EU index: (a) L234A and/or L235A; (b) A327G, A330S, and/or P331S; (c) E233P, L234V, L235A, and/or G236del; (d) E233P, L234V, and/or L235A; (e) E233P, L234V, L235A, G236del, A327G, A330S, and/or P331S; (f) E233P, L234V, L235A, A327G, A330S, and/or P331S; (g) N297A; (h) N297G; (i) N297Q; (j) L242C, N297C, and/or K334C; (k) A287C, N297G, and/or L306C; (l) R292C, N297G, and/or V302C; (m) N297G, V323C, and/or I332C; (n) V259C, N297G, and/or L306C; (o) L234F, L235Q, K322Q, M252Y, S254T, and/or T256E; (p) L234A, L235A, and/or P329G; or (q) L234A, L235Q, and K322Q. In some embodiments, the first and/or second CH region is a human IgG2 CH comprising one of the following mutation(s), wherein the numbering of the constant region is as per the EU index: (a) A330S and/or P331S; (b) V234A, G237A, P238S, H268A, V309L, A330S, and/or P331S; or (c) V234A, G237A, H268Q, V309L, A330S, P331S, C232S, C233S, S267E, L328F, M252Y, S254T, and/or T256E. In some embodiments, the first and/or second CH region is a human IgG4 CH comprising one of the following mutation(s), wherein the numbering of the constant region is as per the EU index: (a) E233P, F234V, L235A, and/or G236del; (b) E233P, F234V, and/or L235A; (c) S228P and/or L235E; or (d) S228P and/or L235A.

In some embodiments, the first and/or second CH region comprise one or more mutations that enhance effector function. In some embodiments, the first and/or second CH region is a human IgG1 CH comprising one of the following mutation(s), wherein the numbering of the constant region is as per the EU index: (a) F243L, R292P, Y300L, V305I, and/or P396L; (b) S239D and/or I332E; (c) S239D, I332E, and/or A330L; (d) S298A, E333A, and/or K334A; (e) G236A, S239D, and/or I332E; (f) K326W and/or E333S; (g) S267E, H268F, and/or S324T; or (h) E345R, E430G, and/or S440Y.

In some embodiments, at least one of the first and second heavy chains is non-fucosylated. In some embodiments, the antibody or antigen-binding fragment thereof is produced in a cell line (a) having an alpha-1,6-fucosyltransferase (Fut8) knockout; or (b) line overexpressing β1,4-N-acetylglucosaminyltransferase III (GnT-III) and optionally overexpressing Golgi μ-mannosidase II (ManII).

In some embodiments, the first and/or second CL region is a human kappa constant region.

In some embodiments,
(a) (i) the first antigen-binding domain comprises:
   a VH region comprising HCDR1, HCDR2, and HCDR3 having the amino acid sequence of SEQ ID NOs: 3, 21, and 5, respectively; and
   a VL region comprising LCDR1, LCDR2, and LCDR3 having the amino acid sequence of SEQ ID NOs: 32, 12, and 13, respectively; and
   (ii) the second antigen-binding domain comprises:
   a VH region comprising HCDR1, HCDR2, and HCDR3 having the amino acid sequence of SEQ ID NOs: 41, 53, and 43, respectively; and
   a VL region comprising LCDR1, LCDR2, and LCDR3 having the amino acid sequence of SEQ ID NOs: 46, 47, and 48, respectively; or
(b) (i) the first antigen-binding domain comprises:
   a VH region comprising HCDR1, HCDR2, and HCDR3 having the amino acid sequence of SEQ ID NOs: 122, 123, and 124, respectively; and
   a VL region comprising LCDR1, LCDR2, and LCDR3 having the amino acid sequence of SEQ ID NOs: 127, 128, and 129, respectively; and
   (ii) the second antigen-binding domain comprises:
   a VH region comprising HCDR1, HCDR2, and HCDR3 having the amino acid sequence of SEQ ID NOs: 41, 53, and 43, respectively; and
   a VL region comprising LCDR1, LCDR2, and LCDR3 having the amino acid sequence of SEQ ID NOs: 46, 47, and 48, respectively; or

In some embodiments, (a) the first antigen-binding domain comprises a VH region having the amino acid sequence of SEQ ID NO: 25, and a VL region having the amino acid sequence of SEQ ID NO: 38; and the second antigen-binding domain comprises a VH region having the amino acid sequence of SEQ ID NO: 52, and a VL region having the amino acid sequence of SEQ ID NO: 87; or
(b) the first antigen-binding domain comprises a VH region having the amino acid sequence of SEQ ID NO: 121, and a VL region having the amino acid sequence of SEQ ID NO: 126; and the second antigen-binding domain comprises a VH region having the amino acid sequence of SEQ ID NO: 52, and a VL region having the amino acid sequence of SEQ ID NO: 87.

In some embodiments, the antigen-binding fragment is a Fab, a Fab′, a Fab2, a F(ab′)2, Fv, a single-chain Fv (scFv), or a diabody.

In some embodiments, the antibody or antigen-binding fragment thereof is conjugated to an agent. In some embodiments, agent is a cytotoxic agent or label.

In another aspect, provided are compositions comprising the antibody or antigen-binding fragment thereof described above. In some embodiments, the antibody or antigen-binding fragment comprises an Fc region and N-glycoside-linked carbohydrate chains linked to the Fc region, optionally wherein less than 50% of the N-glycoside-linked carbohydrate chains contain a fucose residue. In some embodiments, substantially none of the N-glycoside-linked carbohydrate chains contain a fucose residue.

In another aspect, provided are antibodies or antigen binding fragments thereof that is capable of binding to siglec-6, comprising:
(a) a heavy chain variable (VH) region comprising:
   heavy chain complementarity-determining region 1 (HCDR1) having the amino acid sequence of SEQ ID NO: 133, 141, or 149, or a sequence differing in 1 or 2 amino acids therefrom,
   HCDR2 having the amino acid sequence of SEQ ID NO: 134, 142, 150, or 160, or a sequence differing in 1 or 2 amino acids therefrom, and
   HCDR3 having the amino acid sequence of SEQ ID NO: 135, 143, or 151, or a sequence differing in 1 or 2 amino acids therefrom; and
(b) a light chain variable (VL) region comprising:
   light chain complementarity-determining region 1 (LCDR1) having the amino acid sequence of SEQ ID NO: 137, 145, 153, or 165, or a sequence differing in 1 or 2 amino acids therefrom,
   LCDR2 having the amino acid sequence of SEQ ID NO: 138, 146, or 154, or a sequence differing in 1 or 2 amino acids therefrom, and
   LCDR3 having the amino acid sequence of SEQ ID NO: 139, 147, or 155, or a sequence differing in 1 or 2 amino acids therefrom.

In some embodiments, the antibody or antigen binding fragment comprises:
(a) a VH region comprising:
   HCDR1 having the amino acid sequence of SEQ ID NO: 133, 141, or 149,
   HCDR2 having the amino acid sequence of SEQ ID NO: 134, 142, 150, or 160, and
   HCDR3 having the amino acid sequence of SEQ ID NO: 135, 143, or 151; and
(b) a VL region comprising:
   LCDR1 having the amino acid sequence of SEQ ID NO: 137, 145, 153, or 165,
   LCDR2 having the amino acid sequence of SEQ ID NO: 138, 146, or 154, and
   LCDR3 having the amino acid sequence of SEQ ID NO: 139, 147, or 155.

In some embodiments, the antibody or antigen binding fragment comprises:
(a) (i) a VH region comprising:
   HCDR1 having the amino acid sequence of SEQ ID NO: 133, or a sequence differing in 1 or 2 amino acids therefrom,
   HCDR2 having the amino acid sequence of SEQ ID NO: 134, or a sequence differing in 1 or 2 amino acids therefrom, and
   HCDR3 having the amino acid sequence of SEQ ID NO: 135, or a sequence differing in 1 or 2 amino acids therefrom; and
   (ii) a VL region comprising:
   LCDR1 having the amino acid sequence of SEQ ID NO: 137, or a sequence differing in 1 or 2 amino acids therefrom,
   LCDR2 having the amino acid sequence of SEQ ID NO: 138, or a sequence differing in 1 or 2 amino acids therefrom, and
   LCDR3 having the amino acid sequence of SEQ ID NO: 139, or a sequence differing in 1 or 2 amino acids therefrom; or
(b) (i) a VH region comprising:
   HCDR1 having the amino acid sequence of SEQ ID NO: 141, or a sequence differing in 1 or 2 amino acids therefrom,
   HCDR2 having the amino acid sequence of SEQ ID NO: 142, or a sequence differing in 1 or 2 amino acids therefrom, and
   HCDR3 having the amino acid sequence of SEQ ID NO: 143, or a sequence differing in 1 or 2 amino acids therefrom; and
   (ii) a VL region comprising:
   LCDR1 having the amino acid sequence of SEQ ID NO: 145, or a sequence differing in 1 or 2 amino acids therefrom,
   LCDR2 having the amino acid sequence of SEQ ID NO: 146, or a sequence differing in 1 or 2 amino acids therefrom, and
   LCDR3 having the amino acid sequence of SEQ ID NO: 147, or a sequence differing in 1 or 2 amino acids therefrom; or
(c) (i) a VH region comprising:
   HCDR1 having the amino acid sequence of SEQ ID NO: 149, or a sequence differing in 1 or 2 amino acids therefrom,
   HCDR2 having the amino acid sequence of SEQ ID NO: 150 or 160, or a sequence differing in 1 or 2 amino acids therefrom, and
   HCDR3 having the amino acid sequence of SEQ ID NO: 151, or a sequence differing in 1 or 2 amino acids therefrom; and
   (ii) a VL region comprising:
   LCDR1 having the amino acid sequence of SEQ ID NO: 153 or 165, or a sequence differing in 1 or 2 amino acids therefrom,
   LCDR2 having the amino acid sequence of SEQ ID NO: 154, or a sequence differing in 1 or 2 amino acids therefrom, and
   LCDR3 having the amino acid sequence of SEQ ID NO: 155, or a sequence differing in 1 or 2 amino acids therefrom.

In some embodiments, the antibody or antigen binding fragment comprises:
(a) (i) a VH region comprising:
   HCDR1 having the amino acid sequence of SEQ ID NO: 133,
   HCDR2 having the amino acid sequence of SEQ ID NO: 134, and
   HCDR3 having the amino acid sequence of SEQ ID NO: 135; and
   (ii) a VL region comprising:
   LCDR1 having the amino acid sequence of SEQ ID NO: 137,
   LCDR2 having the amino acid sequence of SEQ ID NO: 138, and
   LCDR3 having the amino acid sequence of SEQ ID NO: 139; or
(b) (i) a VH region comprising:
   HCDR1 having the amino acid sequence of SEQ ID NO: 141,
   HCDR2 having the amino acid sequence of SEQ ID NO: 142, and
   HCDR3 having the amino acid sequence of SEQ ID NO: 143; and
   (ii) a VL region comprising:
   LCDR1 having the amino acid sequence of SEQ ID NO: 145,
   LCDR2 having the amino acid sequence of SEQ ID NO: 146, and
   LCDR3 having the amino acid sequence of SEQ ID NO: 147; or
(c) (i) a VH region comprising:
   HCDR1 having the amino acid sequence of SEQ ID NO: 149,
   HCDR2 having the amino acid sequence of SEQ ID NO: 150 or 160, and
   HCDR3 having the amino acid sequence of SEQ ID NO: 151; and
   (ii) a VL region comprising:
   LCDR1 having the amino acid sequence of SEQ ID NO: 153 or 165,
   LCDR2 having the amino acid sequence of SEQ ID NO: 154, and
   LCDR3 having the amino acid sequence of SEQ ID NO: 155.

In some embodiments, the antibody or antigen binding fragment comprises:
(a) a VH region comprising:
   (i) HCDR1, HCDR2, and HCDR3 having the amino acid sequence of SEQ ID NOs: 133, 134, and 135, respectively;
   (ii) HCDR1, HCDR2, and HCDR3 having the amino acid sequence of SEQ ID NOs: 141, 142, and 143, respectively;
   (iii) HCDR1, HCDR2, and HCDR3 having the amino acid sequence of SEQ ID NOs: 149, 150, and 151, respectively; or
   (iv) HCDR1, HCDR2, and HCDR3 having the amino acid sequence of SEQ ID NOs: 149, 160, and 151, respectively; and
(b) a VL region comprising:
   (i) LCDR1, LCDR2, and LCDR3 having the amino acid sequence of SEQ ID NOs: 137, 138, and 139, respectively;
   (ii) LCDR1, LCDR2, and LCDR3 having the amino acid sequence of SEQ ID NOs: 145, 146, and 147, respectively;
   (iii) LCDR1, LCDR2, and LCDR3 having the amino acid sequence of SEQ ID NOs: 153, 154, and 155, respectively; or
   (iv) LCDR1, LCDR2, and LCDR3 having the amino acid sequence of SEQ ID NOs: 165, 154, and 155, respectively.

In some embodiments, the VH region comprises an amino acid sequence having at least 70%, at least 80%, at least 90%, or at least 95% sequence identity to the amino acid sequence of SEQ ID NO: 132, 140, 148, 159, or 162; and the VL region comprises an amino acid sequence having at least 70%, at least 80%, at least 90%, or at least 95% sequence identity to the amino acid sequence of SEQ ID NO: 136, 144, 152, 164, 167, or 169.

In some embodiments, the VH region comprises the amino acid sequence of SEQ ID NO: 132, 140, 148, 159, or 162; and the VL region comprises the amino acid sequence of SEQ ID NO: 136, 144, 152, 164, 167, or 169.

In some embodiments, (a) the VH region comprises the amino acid sequence of SEQ ID NO: 132, and the VL region comprises the amino acid sequence of SEQ ID NO: 139; (b) the VH region comprises the amino acid sequence of SEQ ID NO: 140, and the VL region comprises the amino acid sequence of SEQ ID NO: 144; or (c) the VH region comprises the amino acid sequence of SEQ ID NO: 148, and the VL region comprises the amino acid sequence of SEQ ID NO: 152.

In some embodiments, (a) the VH region comprises the amino acid sequence of SEQ ID NO: 159, and the VL region comprises the amino acid sequence of SEQ ID NO: 164; (b) the VH region comprises the amino acid sequence of SEQ ID NO: 162, and the VL region comprises the amino acid sequence of SEQ ID NO: 167; or (c) the VH region comprises the amino acid sequence of SEQ ID NO: 162, and the VL region comprises the amino acid sequence of SEQ ID NO: 169.

In some embodiments, the antibody is a humanized antibody.

In another aspect, provided are antibodies or antigen binding fragments thereof that is capable of binding to siglec-6, comprising:
(a) a heavy chain variable domain (VH) comprising:
   heavy chain complementarity-determining region 1 (HCDR1) having the amino acid sequence of SEQ ID NO: 3,
   HCDR2 having the amino acid sequence of SEQ ID NO: 21, and
   HCDR3 having the amino acid sequence of SEQ ID NO: 5; and
(a) a light chain variable domain (VL) comprising:
   light chain complementarity-determining region 1 (LCDR1) having the amino acid sequence of SEQ ID NO: 32,
   LCDR2 having the amino acid sequence of SEQ ID NO: 12, and
   LCDR3 having the amino acid sequence of SEQ ID NO: 13.

In some embodiments, the VH region comprises an amino acid sequence having at least 70%, at least 80%, at least 90%, or at least 95% sequence identity to the amino acid sequence of SEQ ID NO: 20, 23, 25, 27, or 29; and the VL region comprises an amino acid sequence having at least 70%, at least 80%, at least 90%, or at least 95% sequence identity to the amino acid sequence of SEQ ID NO: 31, 36, or 38.

In some embodiments, the antibody or antigen binding fragment comprises: the VH region comprises the amino acid sequence of SEQ ID NO: 20, 23, 25, 27, or 29; and the VL region comprises the amino acid sequence of SEQ ID NO: 31, 36, or 38.

In some embodiments, (a) the VH region comprises the amino acid sequence of SEQ ID NO: 23, and the VL region comprises the amino acid sequence of SEQ ID NO: 38;
(b) the VH region comprises the amino acid sequence of SEQ ID NO: 25, and the VL region comprises the amino acid sequence of SEQ ID NO: 38;
(c) the VH region comprises the amino acid sequence of SEQ ID NO: 27, and the VL region comprises the amino acid sequence of SEQ ID NO: 38; or
(d) the VH region comprises the amino acid sequence of SEQ ID NO: 29, and the VL region comprises the amino acid sequence of SEQ ID NO: 38.

In some embodiments, the VH region comprises the amino acid sequence of SEQ ID NO: 25, and the VL region comprises the amino acid sequence of SEQ ID NO: 38.

In some embodiments, the antibody or antigen binding fragment is capable of binding siglec-6 with a K_{D} value of at most about 10 nM, at most about 5 nM, or at most about 1 nM. In some embodiments, the antibody or antigen binding fragment thereof (a) is capable of binding human siglec-6; (b) is capable of binding cynomolgus siglec-6; (c) is capable of binding a mast cell, optionally preferentially than another immune cell (e.g., T cell, dendritic cell, macrophage, neutrophil, or basophil); (d) does not induce mast cell degranulation; and/or (e) has a comparable or lower internalization rate compared to a reference antibody capable of binding siglect-6, optionally wherein the reference antibody comprises a VH region comprising the amino acid sequence of SEQ ID NO: 2 and a VL region comprising the amino acid sequence of SEQ ID NO: 10.

In another aspect, provided are humanized antibodies or antigen binding fragments thereof that is capable of binding to c-Kit, comprising:
(a) a heavy chain variable domain (VH) comprising:
   heavy chain complementarity-determining region 1 (HCDR1) having the amino acid sequence of SEQ ID NO: 41, or a sequence differing in 1 amino acid therefrom,
   HCDR2 having the amino acid sequence of SEQ ID NO: 53, or a sequence differing in 1 amino acid therefrom, and
   HCDR3 having the amino acid sequence of SEQ ID NO: 43, or a sequence differing in 1 amino acid therefrom; and
(a) a light chain variable domain (VL) comprising:
   light chain complementarity-determining region 1 (LCDR1) having the amino acid sequence of SEQ ID NO: 46, or a sequence differing in 1 amino acid therefrom,
   LCDR2 having the amino acid sequence of SEQ ID NO: 47, or a sequence differing in 1 amino acid therefrom, and
   LCDR3 having the amino acid sequence of SEQ ID NO: 48, or a sequence differing in 1 amino acid therefrom.

In some embodiments, the antibody or antigen binding fragment comprises:
(a) a VH region comprising:
   HCDR1 having the amino acid sequence of SEQ ID NO: 41,
   HCDR2 having the amino acid sequence of SEQ ID NO: 53, and
   HCDR3 having the amino acid sequence of SEQ ID NO: 43; and
(b) a VL region comprising:
   LCDR1 having the amino acid sequence of SEQ ID NO: 46,
   LCDR2 having the amino acid sequence of SEQ ID NO: 47, and
   LCDR3 having the amino acid sequence of SEQ ID NO: 48.

In some embodiments, the VH comprises:
(a) HCDR1 having the amino acid sequence of SEQ ID NO: 60, 61, or 62,
(b) HCDR2 having the amino acid sequence of SEQ ID NO: 63, 64, 65, 66, 67, or 68, and
(c) HCDR3 having the amino acid sequence of SEQ ID NO: 69, 70, 71, or 72.

In some embodiments, the VL comprises:
(a) LCDR1 having the amino acid sequence of SEQ ID NO: 90, 91, 92, 93, 94, or 95,
(b) LCDR2 having the amino acid sequence of SEQ ID NO: 96 or 97, and
(c) LCDR3 having the amino acid sequence of SEQ ID NO: 98, 99, 100, 101, 102, 103, or 104.

In some embodiments,
(a) the VH region comprises:
   (i) HCDR1, HCDR2, and HCDR3 having the amino acid sequence of SEQ ID NOs: 41, 53, and 43, respectively;
   (ii) HCDR1, HCDR2, and HCDR3 having the amino acid sequence of SEQ ID NOs: 60, 53, and 43, respectively;
   (iii) HCDR1, HCDR2, and HCDR3 having the amino acid sequence of SEQ ID NOs: 61, 53, and 43, respectively;
   (iv) HCDR1, HCDR2, and HCDR3 having the amino acid sequence of SEQ ID NOs: 62, 53, and 43, respectively;
   (v) HCDR1, HCDR2, and HCDR3 having the amino acid sequence of SEQ ID NOs: 41, 63, and 43, respectively;
   (vi) HCDR1, HCDR2, and HCDR3 having the amino acid sequence of SEQ ID NOs: 41, 64, and 43, respectively;
   (vii) HCDR1, HCDR2, and HCDR3 having the amino acid sequence of SEQ ID NOs: 41, 65, and 43, respectively;
   (viii) HCDR1, HCDR2, and HCDR3 having the amino acid sequence of SEQ ID NOs: 41, 66, and 43, respectively;
   (ix) HCDR1, HCDR2, and HCDR3 having the amino acid sequence of SEQ ID NOs: 41, 67, and 43, respectively;
   (x) HCDR1, HCDR2, and HCDR3 having the amino acid sequence of SEQ ID NOs: 41, 68, and 43, respectively;
   (xi) HCDR1, HCDR2, and HCDR3 having the amino acid sequence of SEQ ID NOs: 41, 53, and 69, respectively;
   (xii) HCDR1, HCDR2, and HCDR3 having the amino acid sequence of SEQ ID NOs: 41, 53, and 70, respectively;
   (xiii) HCDR1, HCDR2, and HCDR3 having the amino acid sequence of SEQ ID NOs: 41, 53, and 71, respectively; or
   (xiv) HCDR1, HCDR2, and HCDR3 having the amino acid sequence of SEQ ID NOs: 41, 53, and 72, respectively; and
(b) the VL region comprising:
   (i) LCDR1, LCDR2, and LCDR3 having the amino acid sequence of SEQ ID NOs: 46, 47, and 48, respectively;
   (ii) LCDR1, LCDR2, and LCDR3 having the amino acid sequence of SEQ ID NOs: 90, 47, and 48, respectively;
   (iii) LCDR1, LCDR2, and LCDR3 having the amino acid sequence of SEQ ID NOs: 91, 47, and 48, respectively;
   (iv) LCDR1, LCDR2, and LCDR3 having the amino acid sequence of SEQ ID NOs: 92, 47, and 48, respectively;
   (v) LCDR1, LCDR2, and LCDR3 having the amino acid sequence of SEQ ID NOs: 93, 47, and 48, respectively;
   (vi) LCDR1, LCDR2, and LCDR3 having the amino acid sequence of SEQ ID NOs: 94, 47, and 48, respectively;
   (vii) LCDR1, LCDR2, and LCDR3 having the amino acid sequence of SEQ ID NOs: 95, 47, and 48, respectively;
   (viii) LCDR1, LCDR2, and LCDR3 having the amino acid sequence of SEQ ID NOs: 46, 96, and 48, respectively;
   (ix) LCDR1, LCDR2, and LCDR3 having the amino acid sequence of SEQ ID NOs: 46, 97, and 48, respectively;
   (x) LCDR1, LCDR2, and LCDR3 having the amino acid sequence of SEQ ID NOs: 46, 47, and 98, respectively;
   (xi) LCDR1, LCDR2, and LCDR3 having the amino acid sequence of SEQ ID NOs: 46, 47, and 99, respectively;
   (xii) LCDR1, LCDR2, and LCDR3 having the amino acid sequence of SEQ ID NOs: 46, 47, and 100, respectively;
   (xiii) LCDR1, LCDR2, and LCDR3 having the amino acid sequence of SEQ ID NOs: 46, 47, and 101, respectively;
   (xiv) LCDR1, LCDR2, and LCDR3 having the amino acid sequence of SEQ ID NOs: 46, 47, and 102, respectively;
   (xv) LCDR1, LCDR2, and LCDR3 having the amino acid sequence of SEQ ID NOs: 46, 47, and 103, respectively; or
   (xvi) LCDR1, LCDR2, and LCDR3 having the amino acid sequence of SEQ ID NOs: 46, 47, and 104, respectively.

In some embodiments, the VH region comprises an amino acid sequence having at least 70%, at least 80%, at least 90%, or at least 95% sequence identity to the amino acid sequence of SEQ ID NO: 52, 55, 57, 59, 73, 74, 75, 76, 77, 78, 79, 80, 81, 82, 83, 84, or 85; and the VL region comprises an amino acid sequence having at least 70%, at least 80%, at least 90%, or at least 95% sequence identity to the amino acid sequence of SEQ ID NO: 87, 89, 105, 106, 107, 108, 109, 110, 111, 112, 113, 114, 115, 116, 117, 118, or 119.

In some embodiments, the VH region comprises the amino acid sequence of SEQ ID NO: 52, 55, 57, 59, 73, 74, 75, 76, 77, 78, 79, 80, 81, 82, 83, 84, or 85; and the VL region comprises the amino acid sequence of SEQ ID NO: 87, 89, 105, 106, 107, 108, 109, 110, 111, 112, 113, 114, 115, 116, 117, 118, or 119.

In some embodiments,
(a) the VH region comprises the amino acid sequence of SEQ ID NO: 40, and the VL region comprises the amino acid sequence of SEQ ID NO: 45;
(b) the VH region comprises the amino acid sequence of SEQ ID NO: 52, and the VL region comprises the amino acid sequence of SEQ ID NO: 87;
(c) the VH region comprises the amino acid sequence of SEQ ID NO: 55, and the VL region comprises the amino acid sequence of SEQ ID NO: 87;
(d) the VH region comprises the amino acid sequence of SEQ ID NO: 57, and the VL region comprises the amino acid sequence of SEQ ID NO: 87;
(e) the VH region comprises the amino acid sequence of SEQ ID NO: 59, and the VL region comprises the amino acid sequence of SEQ ID NO: 87;
(f) the VH region comprises the amino acid sequence of SEQ ID NO: 52, and the VL region comprises the amino acid sequence of SEQ ID NO: 89; or
(g) the VH region comprises the amino acid sequence of SEQ ID NO: 55, and the VL region comprises the amino acid sequence of SEQ ID NO: 89.

In some embodiments, the VH region comprises the amino acid sequence of SEQ ID NO: 52, and the VL region comprises the amino acid sequence of SEQ ID NO: 87.

In some embodiments, the antibody or antigen binding fragment thereof is capable of binding siglec-6 with a K_{D} value of between about 10 and about 500 nM, between about 20 and about 100 nM; or between about 25 and about 80 nM. In some embodiments, the antibody or antigen binding fragment thereof (a) is capable of binding human c-Kit; (b) is capable of binding cynomolgus c-Kit; and/or (c) has a comparable or lower toxicity compared to a reference antibody capable of binding c-Kit, optionally wherein the reference antibody comprises a VH region comprising the amino acid sequence of SEQ ID NO: 40, and a VL region comprising the amino acid sequence of SEQ ID NO: 45.

In some embodiments, the antigen binding fragment described above is a Fab, a F(ab′)2, a Fab′, a single-chain Fv (scFv), an Fv fragment, a Fd fragment, or a diabody.

In some embodiments, the antibody or antigen binding fragment thereof comprises an antibody heavy chain constant region, optionally wherein the constant region is a human IgG heavy chain constant region, optionally wherein the human IgG heavy chain constant region is an IgG1 heavy chain constant region. In some embodiments, the constant region comprises one or more mutations that reduce or abrogate effector function. In some embodiments, the constant region comprises one or more mutations that enhance effector function. In some embodiments, at least one of the first and second heavy chains is afucosylated (e.g., at least 75% afucosylated).

In some embodiments, the antibody or antigen binding fragment described herein is conjugated to a therapeutic agent or a label.

In another aspects, provided are polynucleotides encoding antibodies or antigen binding fragments thereof described herein. In another aspects, provided are expression vectors comprising the polynucleotide. In another aspects, provided are host cells comprising the polynucleotide or the expression vector. In some embodiments, the host cell is a mammalian or insect cell. In some embodiments, the host cell (a) comprises a Fut8 knockout; and/or (b) overexpresses GnT-III and optionally ManII.

In another aspects, provided are pharmaceutical compositions comprising an antibody or antigen binding fragment thereof described herein.

In another aspects, provided are methods of treating a disease or disorder in a subject in need thereof, comprising administering to the subject an effective amount of an antibody or antigen binding fragment thereof described herein or a pharmaceutical composition described herein. In some embodiments, the disease or disorder is a mast cell associated disease or disorder. In some embodiments, the disease or disorder is an autoimmune disease, an allergy, an inflammation, pruritus, or a cancer. In some embodiments, the disease or disorder is associated with expression of siglec-6 and/or c-Kit.

### BRIEF DESCRIPTION OF THE DRAWINGS

FIG. 1 depicts the inhibition of stem cell factor (SCF)-dependent cell proliferation in M-07e cells by SR1 c-Kit antibodies.

FIG. 2 depicts the inhibition of SCF-dependent cell proliferation in M-07e cells by c-Kit/siglec 6 bispecific antibodies.

FIG. 3 shows that humanized siglec-6 antibody hRND-3 with normal, effector silent, or effector enhanced Fc domains did not induce degranulation of mast cells as measured by β-hexosaminidase release. IFNγ treatment of mast cells to upregulate FcγRI did not lead to degranulation by siglec-6 antibodies. Treatment with FcεRIα crosslinking antibody plus SCF and PMA/Ionomycin were used as positive controls.

### DETAILED DESCRIPTION

### Definitions

The terms “a” and “an” as used herein mean “one or more” and include the plural unless the context is inappropriate.

As used herein, the terms “about,” “approximately,” and “comparable to,” when used herein in reference to a value, refer to a value that is similar to the referenced value in the context of that referenced value. In general, those skilled in the art, familiar with the context, will appreciate the relevant degree of variance encompassed by “about,” “approximately,” and “comparable to” in that context. For example, in some embodiments, the terms “about,” “approximately,” and “comparable to” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

As used herein, the terms “antagonistic,” “neutralizing” or “blocking,” when used in reference to an antibody or antigen-binding fragment thereof, is intended to refer to an antibody or fragment thereof whose binding to its target results in inhibition of at least some of the biological activity of the target.

As used herein, “antibody” refers to a polypeptide whose amino acid sequence includes immunoglobulins and fragments thereof which specifically bind to a designated antigen, or fragments thereof. Antibodies in accordance with the present invention may be of any type (e.g., IgA, IgD, IgE, IgG, or IgM) or subtype (e.g., IgAQ1, IgA2, IgG1, IgG2, IgG3, or IgG4). Those of ordinary skill in the art will appreciate that a characteristic sequence or portion of an antibody may include amino acids found in one or more regions of an antibody (e.g., variable region, hypervariable region, constant region, heavy chain, light chain, and combinations thereof). Moreover, those of ordinary skill in the art will appreciate that a characteristic sequence or portion of an antibody may include one or more polypeptide chains, and may include sequence elements found in the same polypeptide chain or in different polypeptide chains.

As used herein, “antibody mimetic” refers to any molecule that is capable of mimicking an antibody's ability to bind an antigen, but which are not limited to antibody structures. Examples of antibody mimetics include, but are not limited to, Affibodies, Affilins, Affimers, Afftins, Alphabodies, Anticalins, avimers, Centyrins, DARPins, Fynomers, monobodies, and nanoCLAMPs.

An “antigen-binding fragment” of an antibody comprises a portion of an intact antibody, which portion is still capable of antigen binding. In certain embodiments, papain digestion of antibodies produce two identical antigen-binding fragments, called “Fab” fragments, and a residual “Fc” fragment, a designation reflecting the ability to crystallize readily. The Fab fragment consists of an entire light chain along with the variable region domain of the heavy chain (VH), and the first constant domain of one heavy chain (C_{H}1). Each Fab fragment is monovalent with respect to antigen binding, i.e., it has a single antigen-binding site. In certain embodiments, pepsin treatment of an antibody yields a single large F(ab′)₂ fragment which roughly corresponds to two disulfide linked Fab fragments having different antigen-binding activity and that is still capable of cross-linking antigen. Fab′ fragments differ from Fab fragments by having a few additional residues at the carboxy terminus of the C_{H}1 domain, including one or more cysteines from the antibody hinge region. Fab′-SH designates an Fab′ in which the cysteine residue(s) of the constant domains bear a free thiol group. F(ab′)₂ antibody fragments originally were produced as pairs of Fab′ fragments having hinge cysteines between them. Other chemical couplings of antibody fragments are also known.

As used herein, the term “antigen-binding site” refers to any molecule or any part of a molecule that is capable of antigen binding. In human antibodies, the antigen-binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FR.” Thus, the term “FR” refers to amino acid sequences which are naturally found between and adjacent to hypervariable regions in immunoglobulins. In a human antibodies, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In certain animals, such as camels and cartilaginous fish, the antigen-binding site may by formed by a single antibody chain providing a “single domain antibody.” An antigen-binding site can also be or exist in an antibody mimetic.

The CDRs can be determined by the methods described in Kabat et al., J. Biol. Chem. 252, 6609-6616 (1977) and Kabat et al., Sequences of protein of immunological interest. (1991), Chothia et al., J. Mol. Biol. 196:901-917 (1987), and MacCallum et al., J. Mol. Biol. 262:732-745 (1996). The CDRs determined under these definitions typically include overlapping or subsets of amino acid residues when compared against each other. In certain embodiments, the term “CDR” is a CDR as defined by MacCallum et al., J. Mol. Biol. 262:732-745 (1996) and Martin A., Protein Sequence and Structure Analysis of Antibody Variable Domains, in Antibody Engineering, Kontermann and Dubel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of protein of immunological interest. (1991). In certain embodiments, heavy chain CDRs and light chain CDRs of an antibody are defined using different conventions. For example, in certain embodiments, the heavy chain CDRs are defined according to MacCallum (supra), and the light CDRs are defined according to Kabat (supra). CDRH1, CDRH2 and CDRH3 denote the heavy chain CDRs, and CDRL1, CDRL2 and CDRL3 denote the light chain CDRs.

As used herein, “binding affinity” of a molecule of the disclosure (e.g., an antigen-binding site) to a given targets (e.g., FCER1 or c-Kit) can be determined by a multitude of methods known to those skilled in the art. Such methods include, but are not limited to, fluorescence titration, ELISA (enzyme-linked immunosorbent assay), calorimetric methods such as isothermal titration calorimetry (ITC), and surface plasmon resonance (SPR).

As used herein, “bispecific” refers to a molecule is able to specifically bind to at least two distinct targets. Typically, a bispecific molecule comprises two antigen-binding sites, each of which is specific for a different target. In some embodiments, a bispecific molecule is capable of binding two targets simultaneously.

As used herein, unless otherwise specified, “c-Kit” means human c-Kit defined by UniProt P10721, and includes variants, isoforms, and species homologs of human c-Kit. c-Kit is also known as “KIT,” “CD117,” “mast/stem cell growth factor receptor Kit,” “SCFR,” “proto-oncogene c-Kit,” or “tyrosine-protein kinase Kit,” which are used interchangeably.

As used herein, “detectable affinity” refers to the ability to bind to a given target with an affinity constant, typically measured by K_{D} or EC₅₀, of at most about 10⁻⁵ M or lower (a lower K_{D} or EC₅₀ value reflects better binding ability). Lower affinities that are no longer measurable with common methods such as ELISA (enzyme-linked immunosorbent assay) are of secondary importance.

As used herein, the term “epitope” is an antigenic determinant that interacts with a specific antigen binding site in the variable region of an antibody molecule, known as the paratope, and which is comprised of the six complementary-determining regions of the antibody. A single antigen may have more than one epitope. Epitopes may be conformational or linear. A conformational epitope is comprised of spatially juxtaposed amino acids from different segments of a linear polypeptide chain. A linear epitope is comprised of adjacent amino acid residues in a polypeptide chain.

An “Fc” fragment comprises the carboxy-terminal portions of both heavy chains held together by disulfides. The effector functions of antibodies are determined by sequences in the Fc region, the region which is also recognized by Fc receptors (FcR) found on certain types of cells.

A “fragment” with respect to an antigen (e.g., siglec-6 or c-Kit) refers to N-terminally and/or C-terminally truncated or protein domains of the antigen. In some embodiments, a fragment of an antigen retains the capability of the full-length antigen to be recognized and/or bound by an antibody or antigen-binding fragment thereof of the disclosure.

As used herein, “fucosylation” or “fucosylated” refers to the presence of fucose residues within the oligosaccharides attached to the peptide backbone of an antibody. Specifically, a fucosylated antibody comprises α(1,6)-linked fucose at the innermost N-acetylglucosamine (GlcNAc) residue in one or both of the N-linked oligosaccharides attached to the antibody Fc region. “Non-fucosylated,” “afucosylated,” or “fucose-deficient” antibody refers to a glycosylation antibody variant comprising an Fc region wherein a carbohydrate structure attached to the Fc region has reduced fucose or lacks fucose. In some embodiments, non-fucosylated or fucose-deficient antibodies have reduced fucose relative to the amount of fucose on the same antibody produced in a cell line. In some embodiments, an antibody with reduced fucose or lacking fucose has improved ADCC function.

The “degree of fucosylation” is the percentage of fucosylated oligosaccharides relative to all oligosaccharides, which may be identified by methods known in the art, e.g., in an N-glycosidase F treated antibody composition assessed by matrix-assisted laser desorption-ionization time-of-flight mass spectrometry (MALDI-TOF MS). In a composition of a “fully fucosylated antibody,” essentially all oligosaccharides comprise fucose residues, i.e., are fucosylated. In some embodiments, a composition of a fully fucosylated antibody has a degree of fucosylation of at least about 90%. In contrast, in a composition of a “fully non-fucosylated antibody,” essentially none of the oligosaccharides are fucosylated. In some embodiments, a composition of a fully non-fucosylated antibody has a degree of fucosylation of less than about 10%. In a composition of a “partially fucosylated antibody,” only part of the oligosaccharides comprise fucose. An individual antibody in such a composition may comprise fucose residues in none, one or both of the N-linked oligosaccharides in the Fc region, provided that the composition does not comprise essentially all individual antibodies that lack fucose residues in the N-linked oligosaccharides in the Fc region, nor essentially all individual antibodies that contain fucose residues in both of the N-linked oligosaccharides in the Fc region. In one embodiment, a composition of a partially fucosylated antibody has a degree of fucosylation of about 10% to about 80% (e.g., about 50% to about 80%, about 60% to about 80%, or about 70% to about 80%). Assays for measuring degree of fucosylation, as well as methods and cell lines for producing antibodies with altered, reduced, or eliminated fucosylation, are known in the art (see, e.g., WO 2023/044390 A1).

As used herein, the term “monoclonal antibody” refers to an antibody obtained from a population of substantially homogeneous antibodies, e.g., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, monoclonal antibodies may be made by a hybridoma method, or may be made by recombinant DNA methods (see, e.g., U.S. Pat. No. 4,816,567). “Monoclonal antibodies” may also be isolated from phage antibody libraries.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable salt” refers to any pharmaceutically acceptable salt (e.g., acid or base) of a compound described in the present application which, upon administration to a subject, is capable of providing a compound described in this application or an active metabolite or residue thereof. As is known to those of skill in the art, “salts” of the compounds described in the present application may be derived from inorganic or organic acids and bases.

Exemplary acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds described in the present application and their pharmaceutically acceptable acid addition salts.

Exemplary bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Exemplary salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds described in the present application compounded with a suitable cation such as Na⁺, NH₄⁺, and NW₄⁺ (wherein W is a C₁₋₄ alkyl group), and the like.

For therapeutic use, salts of the compounds described in the present application are contemplated as being pharmaceutically acceptable. However, salts of acids and bases that are non-pharmaceutically acceptable may also find use, for example, in the preparation or purification of a pharmaceutically acceptable compound.

As used herein, unless otherwise specified, “siglec-6” means human siglec-6 defined by UniProt 043699, and includes variants, isoforms, and species homologs of human siglec-6. Siglec-6 is also known as “SIGLEC6,” “sialic acid-binding Ig-like lectin 6,” “CD33 antigen-like 1,” “CD33L,” “CD33L1,” “CD33L2,” “CD327,” “CDw327,” “obesity-binding protein 1,” or “OB-BP1,” which are used interchangeably.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the phrases “therapeutically effective amount” and “effective amount” are used interchangeably and refer to an amount effective, at dosages and for periods of time necessary, to achieve a desired therapeutic result. A therapeutically effective amount may vary according to factors such as the type of disease (e.g., cancer), disease state, age, sex, and/or weight of the individual, and the ability of an immunoconjugate (or pharmaceutical composition thereof) to elicit a desired response in the individual. An effective amount may also be an amount for which any toxic or detrimental effects of the immunoconjugate or pharmaceutical composition thereof are outweighed by therapeutically beneficial effects.

As used herein, to “treat” a condition or “treatment” of the condition (e.g., the conditions described herein such as cancer) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition (e.g., of a primary cancer and/or of a secondary metastases); delay or slowing the progress of the disease, disorder, or condition; amelioration or palliation of the disease, disorder, or condition; and remission (whether partial or total), whether detectable or undetectable.

### Antigen-Binding Sites

Antigen-Binding Sites Capable of Binding c-Kit

In another aspect, the present application provides antigen-binding sites that are capable of binding c-Kit.

In some embodiments, provided are antigen-binding sites capable of binding c-Kit, present as antibody mimetics. In some embodiments, provided are antigen-binding sites that are variants of an antigen-binding site capable of binding c-Kit and present as an antibody mimetic described herein, in that such antigen-binding sites comprise an amino acid sequence that is at least 85%, at least 87.5%, at least 90%, at least 91%, at least 92%, at least 93%, at least 94%, at least 95%, at least 96%, at least 97%, at least 98%, or at least 99% identical to the amino acid sequence of the antigen-binding site capable of binding c-Kit and present as an antibody mimetic described herein.

In some embodiments, provided are antigen-binding sites capable of binding c-Kit, present as antibodies (e.g., bispecific antibodies). In some embodiments, provided are antigen-binding sites that are variants of an antigen-binding site capable of binding c-Kit and present as an antibody described herein, in that such antigen-binding sites comprise an amino acid sequence that is at least 85%, at least 87.5%, at least 90%, at least 91%, at least 92%, at least 93%, at least 94%, at least 95%, at least 96%, at least 97%, at least 98%, or at least 99% identical to an amino acid sequence comprised in said antigen-binding site capable of binding c-Kit and present as an antibody described herein.

In some embodiments, provided antigen-binding sites capable of binding c-Kit comprise a heavy chain variable domain (VH) comprising a CDR-H1, CDR-H2, and CDR-H3, and/or a light chain variable domain (VL) comprising a CDR-L1, CDR-L2, and CDR-L3. In some embodiments, provided antigen-binding sites capable of binding c-Kit comprise a VH and a VL, wherein the VH comprises a CDR-H1, CDR-H2, and CDR-H3, and the VL comprises a CDR-L1, CDR-L2, and CDR-L3, and wherein the CDR-H1, CDR-H2, CDR-H3, CDR-L1, CDR-L2, and CDR-L3 are each independently selected from those of a VH or VL described in Table 10. CDRs are determined under Kabat, AbM, Chothia, or any other CDR determination method known in the art, of the VH and VL. In certain embodiments, the antigen-binding site comprises the CDR-H1, CDR-H2, CDR-H3, CDR-L1, CDR-L2, and CDR-L3 each independently selected from those described in Table 10. In some embodiments, provided are antigen-binding sites that are variants of the antigen-binding sites capable of binding c-Kit described herein, in that such antigen-binding sites have CDR sequences that each differ by no more than two amino acid residues (e.g., two or one amino acid residue(s)) from a CDR sequence described in Table 10. In some embodiments, provided are an antigen-binding sites that are variants of the antigen-binding sites capable of binding c-Kit described herein, in that such antigen-binding sites have a set of six CDRs whose sequences collectively differ by no more than two amino acid residues (e.g., two or one amino acid residues) from a set of CDR-H1, CDR-H2, CDR-H3, CDR-L1, CDR-L2, and CDR-L3 selected from Table 10. In some embodiments, provided are an antigen-binding sites that are variants of the antigen-binding sites capable of binding c-Kit described herein, in that such antigen-binding sites have a set of six CDRs whose sequences collectively differ by no more than two amino acid residues (e.g., two or one amino acid residues) from those of an anti-c-Kit having a set of VH and VL selected from Table 10.

In some embodiments, provided antigen-binding sites capable of binding c-Kit comprise a VH sequence as described in Table 10, and a VL sequence as described in Table 10. In some embodiments, provided are antigen-binding sites that are variants of the antigen-binding site capable of binding c-Kit described herein, such as an anti-c-Kit having a set of VH and VL selected from Table 10, in that such antigen-binding site have (1) a VH comprising an amino acid sequence that is at least 85%, at least 87.5%, at least 90%, at least 91%, at least 92%, at least 93%, at least 94%, at least 95%, at least 96%, at least 97%, at least 98%, or at least 99% identical to the amino acid sequence of a VH described in Table 10; and (2) a VL comprising an amino acid sequence that is at least 85%, at least 87.5%, at least 90%, at least 91%, at least 92%, at least 93%, at least 94%, at least 95%, at least 96%, at least 97%, at least 98%, or at least 99% identical to the amino acid sequence of a VL described in Table 10.

In some embodiments, provided antigen-binding sites capable of binding c-Kit comprise a heavy chain sequence as described in Table 10, and a light chain sequence as described in Table 10. In some embodiments, provided are antigen-binding sites that are variants of the antigen-binding site capable of binding c-Kit described herein, such as an anti-c-Kit having a set of heavy chain and light chain selected from Table 10, in that such antigen-binding site have (1) a heavy chain comprising an amino acid sequence that is at least 85%, at least 87.5%, at least 90%, at least 91%, at least 92%, at least 93%, at least 94%, at least 95%, at least 96%, at least 97%, at least 98%, or at least 99% identical to the amino acid sequence of a heavy chain described in Table 10; and (2) a light chain comprising an amino acid sequence that is at least 85%, at least 87.5%, at least 90%, at least 91%, at least 92%, at least 93%, at least 94%, at least 95%, at least 96%, at least 97%, at least 98%, or at least 99% identical to the amino acid sequence of a light chain described in Table 10.

In certain embodiments, an antigen-binding site described in the present application is derived from murine SR1. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 40, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 45. In certain embodiments, the antigen-binding site comprises the CDR-H1, CDR-H2, CDR-H3, CDR-L1, CDR-L2, and CDR-L3, determined under Kabat, AbM, Chothia, or any other CDR determination method known in the art, of the VH and VL sequences of SEQ ID NOs: 40 and 45, respectively. In certain embodiments, the VH comprises CDR-H1, CDR-H2, and CDR-H3, comprising the amino acid sequences of SEQ ID NOs: 41, 42, and 43, respectively. In certain embodiments, the VL comprises CDR-L1, CDR-L2, and CDR-L3 comprising the amino acid sequences of SEQ ID NOs: 46, 47, and 48, respectively. In certain embodiments, the antigen-binding site comprises (a) a VH that comprises CDR1, CDR2, and CDR3 comprising the amino acid sequences of SEQ ID NOs: 41, 42, and 43, respectively; and (b) a VL that comprises CDR1, CDR2, and CDR3 comprising the amino acid sequences of SEQ ID NOs: 46, 47, and 48, respectively.

In certain embodiments, an antigen-binding site described in the present application is derived from humanized SR1-1. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 52, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 87. In certain embodiments, the VH comprises an amino acid sequence of SEQ ID NO: 52, 55, 57, 59, 73, 74, 75, 76, 77, 78, 79, 80, 81, 82, 83, 84, or 85. In certain embodiments, the VL comprises an amino acid sequence of SEQ ID NO: 87, 89, 105, 106, 107, 108, 109, 110, 111, 112, 113, 114, 115, 116, 117, 118, or 119. In certain embodiments, the antigen-binding site comprises the CDR-H1, CDR-H2, CDR-H3, CDR-L1, CDR-L2, and CDR-L3, determined under Kabat, AbM, Chothia, or any other CDR determination method known in the art, of the VH and VL sequences of SEQ ID NOs: 52 and 87, respectively. In certain embodiments, the VH comprises CDR-H1, CDR-H2, and CDR-H3, comprising the amino acid sequences of SEQ ID NOs: 41, 53, and 43, respectively. In certain embodiments, the VL comprises CDR-L1, CDR-L2, and CDR-L3 comprising the amino acid sequences of SEQ ID NOs: 46, 47, and 48, respectively. In certain embodiments, the antigen-binding site comprises (a) a VH that comprises CDR-H1, CDR-H2, and CDR-H3 comprising the amino acid sequences of SEQ ID NOs: 41, 53, and 43, respectively; and (b) a VL that comprises CDR-L1, CDR-L2, and CDR-L3 comprising the amino acid sequences of SEQ ID NOs: 46, 47, and 48, respectively.

In certain embodiments, a humanized SR1-1 variant comprises CDR-H1, CDR-H2, CDR-H3, CDR-L1, CDR-L2, and CDR-L3, the sequence of each of which differs by no more than one amino from CDR-H1, CDR-H2, CDR-H3, CDR-L1, CDR-L2, and CDR-L3, determined under Kabat, AbM, Chothia, or any other CDR determination method known in the art, of the VH and VL sequences of SEQ ID NOs: 52 and 87, respectively. In certain embodiments, the VH comprises CDR-H1, CDR-H2, and CDR-H3, the sequence of each of which differs by no more than one amino from SEQ ID NOs: 41, 53, and 43, respectively. In certain embodiments, the VL comprises CDR-L1, CDR-L2, and CDR-L3, the sequence of each of which differs by no more than one amino from SEQ ID NOs: 46, 47, and 48, respectively. In certain embodiments, a humanized SR1-1 variant comprises CDR-H1, CDR-H2, CDR-H3, CDR-L1, CDR-L2, and CDR-L3, whose sequences collectively differ by at least one amino acid residue (e.g., one, two, or three) from CDR-H1, CDR-H2, CDR-H3, CDR-L1, CDR-L2, and CDR-L3, determined under Kabat, AbM, Chothia, or any other CDR determination method known in the art, of the VH and VL sequences of SEQ ID NOs: 52 and 87, respectively. In certain embodiments, the VH comprises CDR-H1, CDR-H2, and CDR-H3, whose sequences collectively differ by at least one amino acid residue from SEQ ID NOs: 41, 53, and 43, respectively. In certain embodiments, the VL comprises CDR-L1, CDR-L2, and CDR-L3, whose sequences collectively differ by at least one amino acid residue from SEQ ID NOs: 46, 47, and 48, respectively. In certain embodiments, CDR-H1 comprises the amino acid sequence of SEQ ID NO: 60, 61, or 62. In certain embodiments, CDR-H2 comprises the amino acid sequence of SEQ ID NO: 63, 64, 65, 66, 67, or 68. In certain embodiments, CDR-H3 comprises the amino acid sequence of SEQ ID NO: 69, 70, 71, or 72. In certain embodiments, CDR-L1 comprises the amino acid sequence of SEQ ID NO: 90, 91, 92, 93, 94, or 95. In certain embodiments, CDR-L2 comprises the amino acid sequence of SEQ ID NO: 96 or 97. In certain embodiments, CDR-L3 comprises the amino acid sequence of SEQ ID NO: 98, 99, 100, 101, 102, 103, or 104.

In certain embodiments, an antigen-binding site described in the present application is derived from humanized SR1-2. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 55, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 87. In certain embodiments, the antigen-binding site comprises the CDR-H1, CDR-H2, CDR-H3, CDR-L1, CDR-L2, and CDR-L3, determined under Kabat, AbM, Chothia, or any other CDR determination method known in the art, of the VH and VL sequences of SEQ ID NOs: 55 and 87, respectively.

In certain embodiments, an antigen-binding site described in the present application is derived from humanized SR1-3. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 57, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 87. In certain embodiments, the antigen-binding site comprises the CDR-H1, CDR-H2, CDR-H3, CDR-L1, CDR-L2, and CDR-L3, determined under Kabat, AbM, Chothia, or any other CDR determination method known in the art, of the VH and VL sequences of SEQ ID NOs: 57 and 87, respectively.

In certain embodiments, an antigen-binding site described in the present application is derived from humanized SR1-4. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 59, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 87. In certain embodiments, the antigen-binding site comprises the CDR-H1, CDR-H2, CDR-H3, CDR-L1, CDR-L2, and CDR-L3, determined under Kabat, AbM, Chothia, or any other CDR determination method known in the art, of the VH and VL sequences of SEQ ID NOs: 59 and 87, respectively.

In certain embodiments, an antigen-binding site described in the present application is derived from humanized SR1-5. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 52, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 89. In certain embodiments, the antigen-binding site comprises the CDR-H1, CDR-H2, CDR-H3, CDR-L1, CDR-L2, and CDR-L3, determined under Kabat, AbM, Chothia, or any other CDR determination method known in the art, of the VH and VL sequences of SEQ ID NOs: 52 and 89, respectively.

In certain embodiments, an antigen-binding site described in the present application is derived from humanized SR1-6. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 55, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 89. In certain embodiments, the antigen-binding site comprises the CDR-H1, CDR-H2, CDR-H3, CDR-L1, CDR-L2, and CDR-L3, determined under Kabat, AbM, Chothia, or any other CDR determination method known in the art, of the VH and VL sequences of SEQ ID NOs: 55 and 89, respectively.

In each of the foregoing embodiments, it is contemplated herein that the VH and/or VL sequences that bind c-Kit may contain amino acid alterations (e.g., at least 1, 2, 3, 4, 5, or 10 amino acid substitutions, deletions, or additions) in the framework regions of the VH and/or VL without affecting their ability to c-Kit. For example, it is contemplated herein that VH and VL sequences that bind c-Kit may contain cysteine heterodimerization mutations, facilitating formation of a disulfide bridge between the VH and VL to form an scFv.

In certain embodiments, antigen-binding sites disclosed herein bind human c-Kit at a K_{D} value less than or equal to (affinity greater than or equal to) 900 uM, 500 uM, 250 uM, 100 uM, 50 uM, 25 uM, 10 uM, 9 uM, 8 uM, 7 uM, 6 uM, 5 uM, 4 uM, 3 uM, 2 uM, 1 uM, 900 nM, 800 nM, 700 nM, 600 nM, 500 nM, 400 nM, 300 nM, 200 nM, 100 nM, 90 nM, 80 nM, 70 nM, 60 nM, 50 nM, 45 nM, 40 nM, 35 nM, 30 nM, 25 nM, 20 nM, 15 nM, 10 nM, 9 nM, 8 nM, 7 nM, 6 nM, 5 nM, 4 nM, 3 nM, 2 nM, 1 nM, 0.5 nM, 0.4 nM, 0.3 nM, 0.2 nM, 0.1 nM, 90 pM, 80 pM, 70 pM, 60 pM, 50 pM, 45 pM, 40 pM, 35 pM, 30 pM, 25 pM, 20 pM, 15 pM, 10 pM, 9 pM, 8 pM, 7 pM, 6 pM, 5 pM, 4 pM, 3 pM, 2 pM, or 1 pM. In certain embodiments, an antigen-binding site of the present application binds human c-Kit at a K_{D} value higher than or equal to (affinity lower than or equal to) 1 pM, 2 pM, 3 pM, 4 pM, 5 pM, 6 pM, 7 pM, 8 pM, 9 pM 10 pM, 15 pM, 20 pM, 25 pM, 30 pM, 35 pM, 40 pM, 45 pM, 50 pM, 60 pM, 70 pM, 80 pM, 90 pM, 0.1 nM, 0.2 nM, 0.3 nM, 0.4 nM, 0.5 nM, 0.6 nM, 0.7 nM, 0.8 nM, 0.9 nM, 1 nM, 2 nM, 3 nM, 4 nM, 5 nM, 6 nM, 7 nM, 8 nM, 9 nM, 10 nM, 15 nM, 20 nM, 25 nM, 30 nM, 35 nM, 40 nM, 45 nM, 50 nM, 60 nM, 70 nM, 80 nM, 90 nM, 100 nM, 150 nM, 200 nM, 250 nM, 300 nM, 400 nM, 500 nM, 600 nM, 700 nM, 800 nM, 900 nM, 1 uM, 2 uM, 3 uM, 4 uM, 5 uM, 6 uM, 7 uM, 8 uM, 9 uM, 10 uM, 25 uM, 50 uM, 100 uM, 250 uM, 500 uM, or 900 uM. In certain embodiments, the antigen binding site of the present application binds human c-Kit at a K_{D} value in the range of picomolar to micromolar. In certain embodiments, the antigen binding site of the present application binds human c-Kit at a K_{D} value in the range of about 1 pM to about 900 uM, 1 pM to about 500 uM, 1 pM to about 100 uM, 1 pM to about 50 uM, 1 pM to about 10 uM, 1 pM to about 5 uM, 1 pM to about 1 uM, 1 pM to about 500 nM, 1 pM to about 100 nM, 1 pM to about 50 nM, 1 pM to about 10 nM, 1 pM to about 5 nM, 1 pM to about 1 nM, 1 pM to about 500 pM, 1 pM to about 100 pM, 1 pM to about 50 pM, 1 pM to about 10 pM, 1 pM to about 5 pM, 5 pM to about 900 uM, 5 pM to about 500 uM, 5 pM to about 100 uM, 5 pM to about 50 uM, 5 pM to about 10 uM, 5 pM to about 5 uM, 5 pM to about 1 uM, 5 pM to about 500 nM, 5 pM to about 100 nM, 5 pM to about 50 nM, 5 pM to about 10 nM, 5 pM to about 5 nM, 5 pM to about 1 nM, 5 pM to about 500 pM, 5 pM to about 100 pM, 5 pM to about 50 pM, 5 pM to about 10 pM, 10 pM to about 900 uM, 10 pM to about 500 uM, 10 pM to about 100 uM, 10 pM to about 50 uM, 10 pM to about 10 uM, 10 pM to about 5 uM, 10 pM to about 1 uM, 10 pM to about 500 nM, 10 pM to about 100 nM, 10 pM to about 50 nM, 10 pM to about 10 nM, 10 pM to about 5 nM, 10 pM to about 1 nM, 10 pM to about 500 pM, 10 pM to about 100 pM, 10 pM to about 50 pM, 50 pM to about 900 uM, 50 pM to about 500 uM, 50 pM to about 100 uM, 50 pM to about 50 uM, 50 pM to about 10 uM, 50 pM to about 5 uM, 50 pM to about 1 uM, 50 pM to about 500 nM, 50 pM to about 100 nM, 50 pM to about 50 nM, 50 pM to about 10 nM, 50 pM to about 5 nM, 50 pM to about 1 nM, 50 pM to about 500 pM, 50 pM to about 100 pM, 100 pM to about 900 uM, 100 pM to about 500 uM, 100 pM to about 100 uM, 100 pM to about 50 uM, 100 pM to about 10 uM, 100 pM to about 5 uM, 100 pM to about 1 uM, 100 pM to about 500 nM, 100 pM to about 100 nM, 100 pM to about 50 nM, 100 pM to about 10 nM, 100 pM to about 5 nM, 100 pM to about 1 nM, 100 pM to about 500 pM, 500 pM to about 500 uM, 500 pM to about 100 uM, 500 pM to about 50 uM, 500 pM to about 10 uM, 500 pM to about 5 uM, 500 pM to about 1 uM, 500 pM to about 500 nM, 500 pM to about 100 nM, 500 pM to about 50 nM, 500 pM to about 10 nM, 500 pM to about 5 nM, 500 pM to about 1 nM, 1 nM to about 500 uM, 1 nM to about 100 uM, 1 nM to about 50 uM, 1 nM to about 10 uM, 1 nM to about 5 uM, 1 nM to about 1 uM, 1 nM to about 500 nM, 1 nM to about 100 nM, 1 nM to about 50 nM, 1 nM to about 10 nM, 1 nM to about 5 nM, 5 nM to about 100 uM, 5 nM to about 50 uM, 5 nM to about 10 uM, 5 nM to about 5 uM, 5 nM to about 1 uM, 5 nM to about 500 nM, 5 nM to about 100 nM, 5 nM to about 50 nM, 5 nM to about 10 nM, 10 nM to about 10 uM, 10 nM to about 5 uM, 10 nM to about 1 uM, 10 nM to about 500 nM, 10 nM to about 100 nM, 10 nM to about 50 nM, 50 nM to about 10 uM, 50 nM to about 5 uM, 50 nM to about 1 uM, 50 nM to about 500 nM, 50 nM to about 100 nM, 100 nM to about 10 uM, 100 nM to about 5 uM, 100 nM to about 1 uM, 100 nM to about 500 nM, 500 nM to about 10 uM, 500 nM to about 5 uM, 500 nM to about 1 uM, 1 uM to about 10 uM, 1 uM to about 5 uM, or 5 uM to about 10 uM. In some embodiments, K_{D} values are measured using standard binding assays, for example, bio-layer interferometry.

In some embodiments, antigen-binding sites disclosed herein bind cynomolgus c-Kit at a comparable affinity (K_{D} value) to that of binding human c-Kit. In some embodiments, the affinity of the antigen-binding site to cynomolgus c-Kit relative to its affinity to human c-Kit, as measured in the same assay, is within a 2-fold, 3-fold, 5-fold, or 10-fold difference (either with greater affinity to human c-Kit or with greater affinity to cynomolgus c-Kit).

In some embodiments, antigen-binding sites disclosed herein bind c-Kit expressed on cell surface.

In some embodiments, antigen-binding sites disclosed herein does not significantly bind other c-Kit family members. In some embodiments, antigen-binding sites disclosed herein does not significantly bind PDGFRa, PDGFRb, CSF1R, or FLT3.

### Antigen-Binding Sites Capable of Binding Siglec-6

In another aspect, the present application provides antigen-binding sites that are capable of binding siglec-6.

In some embodiments, provided are antigen-binding sites capable of binding siglec-6, present as antibody mimetics. In some embodiments, provided are antigen-binding sites that are variants of an antigen-binding site capable of binding siglec-6 and present as an antibody mimetic described herein, in that such antigen-binding sites comprise an amino acid sequence that is at least 85%, at least 87.5%, at least 90%, at least 91%, at least 92%, at least 93%, at least 94%, at least 95%, at least 96%, at least 97%, at least 98%, or at least 99% identical to the amino acid sequence of the antigen-binding site capable of binding siglec-6 and present as an antibody mimetic described herein.

In some embodiments, provided are antigen-binding sites capable of binding siglec-6, present as antibodies (e.g., bispecific antibodies). In some embodiments, provided are antigen-binding sites that are variants of an antigen-binding site capable of binding siglec-6 and present as an antibody described herein, in that such antigen-binding sites comprise an amino acid sequence that is at least 85%, at least 87.5%, at least 90%, at least 91%, at least 92%, at least 93%, at least 94%, at least 95%, at least 96%, at least 97%, at least 98%, or at least 99% identical to an amino acid sequence comprised in said antigen-binding site capable of binding siglec-6 and present as an antibody described herein.

In some embodiments, provided antigen-binding sites capable of binding siglec-6 comprise a heavy chain variable domain (VH) comprising a CDR-H1, CDR-H2, and CDR-H3, and/or a light chain variable domain (VL) comprising a CDR-L1, CDR-L2, and CDR-L3.

In some embodiments, provided antigen-binding sites capable of binding siglec-6 comprise a VH and a VL, wherein the VH comprises a CDR-H1, CDR-H2, and CDR-H3, and the VL comprises a CDR-L1, CDR-L2, and CDR-L3, wherein the CDR-H1, CDR-H2, CDR-H3, CDR-L1, CDR-L2, and CDR-L3 are each independently selected from those of a VH or VL described in Table 9. CDRs are determined under Kabat, AbM, Chothia, or any other CDR determination method known in the art, of the VH and VL. In certain embodiments, the antigen-binding site comprises the CDR-H1, CDR-H2, CDR-H3, CDR-L1, CDR-L2, and CDR-L3 each independently selected from those described in Table 9. In some embodiments, provided are antigen-binding sites that are variants of the antigen-binding sites capable of binding siglec-6 described herein, in that such antigen-binding sites have CDR sequences that each differ by no more than two amino acid residues (e.g., two or one amino acid residue(s)) from a CDR sequence described in Table 9. In some embodiments, provided are an antigen-binding sites that are variants of the antigen-binding sites capable of binding siglec-6 described herein, in that such antigen-binding sites have a set of six CDRs whose sequences collectively differ by no more than two amino acid residues (e.g., two or one amino acid residues) from a set of CDR-H1, CDR-H2, CDR-H3, CDR-L1, CDR-L2, and CDR-L3 selected from Table 9. In some embodiments, provided are an antigen-binding sites that are variants of the antigen-binding sites capable of binding siglec-6 described herein, in that such antigen-binding sites have a set of six CDRs whose sequences collectively differ by no more than two amino acid residues (e.g., two or one amino acid residues) from those of an anti-siglec-6 having a set of VH and VL selected from Table 9.

In some embodiments, provided antigen-binding sites capable of binding siglec-6 comprise a VH sequence as described in Table 9, and a VL sequence as described in Table 9. In some embodiments, provided are antigen-binding sites that are variants of the antigen-binding site capable of binding siglec-6 described herein, such as an anti-siglec-6 having a set of VH and VL selected from Table 9, in that such antigen-binding site have (1) a VH comprising an amino acid sequence that is at least 85%, at least 87.5%, at least 90%, at least 91%, at least 92%, at least 93%, at least 94%, at least 95%, at least 96%, at least 97%, at least 98%, or at least 99% identical to the amino acid sequence of a VH described in Table 9; and (2) a VL domain comprising an amino acid sequence that is at least 85%, at least 87.5%, at least 90%, at least 91%, at least 92%, at least 93%, at least 94%, at least 95%, at least 96%, at least 97%, at least 98%, or at least 99% identical to the amino acid sequence of a VL described in Table 9.

In some embodiments, provided antigen-binding sites capable of binding siglec-6 comprise a heavy chain sequence as described in Table 9, and a light chain sequence as described in Table 9. In some embodiments, provided are antigen-binding sites that are variants of the antigen-binding site capable of binding siglec-6 described herein, such as an anti-siglec-6 having a set of heavy chain and light chain selected from Table 9, in that such antigen-binding site have (1) a heavy chain comprising an amino acid sequence that is at least 85%, at least 87.5%, at least 90%, at least 91%, at least 92%, at least 93%, at least 94%, at least 95%, at least 96%, at least 97%, at least 98%, or at least 99% identical to the amino acid sequence of a heavy chain described in Table 9; and (2) a light chain comprising an amino acid sequence that is at least 85%, at least 87.5%, at least 90%, at least 91%, at least 92%, at least 93%, at least 94%, at least 95%, at least 96%, at least 97%, at least 98%, or at least 99% identical to the amino acid sequence of a light chain described in Table 9.

In certain embodiments, an antigen-binding site described in the present application is derived from murine RND. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 2, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 10. In certain embodiments, the antigen-binding site comprises the CDR-H1, CDR-H2, CDR-H3, CDR-L1, CDR-L2, and CDR-L3, determined under Kabat, AbM, Chothia, or any other CDR determination method known in the art, of the VH and VL sequences of SEQ ID NOs: 2 and 10, respectively. In certain embodiments, the VH comprises CDR-H1, CDR-H2, and CDR-H3, comprising the amino acid sequences of SEQ ID NOs: 3, 4, and 5, respectively. In certain embodiments, the VL comprises CDR-L1, CDR-L2, and CDR-L3 comprising the amino acid sequences of SEQ ID NOs: 11, 12, and 13, respectively. In certain embodiments, the antigen-binding site comprises (a) a VH that comprises CDR1, CDR2, and CDR3 comprising the amino acid sequences of SEQ ID NOs: 3, 4, and 5, respectively; and (b) a VL that comprises CDR1, CDR2, and CDR3 comprising the amino acid sequences of SEQ ID NOs: 11, 12, and 13, respectively.

In certain embodiments, an antigen-binding site described in the present application is derived from humanized RND-2. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 23, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 38. In certain embodiments, the antigen-binding site comprises the CDR-H1, CDR-H2, CDR-H3, CDR-L1, CDR-L2, and CDR-L3, determined under Kabat, AbM, Chothia, or any other CDR determination method known in the art, of the VH and VL sequences of SEQ ID NOs: 23 and 38, respectively. In certain embodiments, the VH comprises CDR-H1, CDR-H2, and CDR-H3, comprising the amino acid sequences of SEQ ID NOs: 3, 21, and 5, respectively. In certain embodiments, the VL comprises CDR-L1, CDR-L2, and CDR-L3 comprising the amino acid sequences of SEQ ID NOs: 32, 12, and 13, respectively. In certain embodiments, the antigen-binding site comprises (a) a VH that comprises CDR1, CDR2, and CDR3 comprising the amino acid sequences of SEQ ID NOs: 3, 21, and 5, respectively; and (b) a VL that comprises CDR1, CDR2, and CDR3 comprising the amino acid sequences of SEQ ID NOs: 32, 12, or 13, respectively.

In certain embodiments, an antigen-binding site described in the present application is derived from humanized RND-3. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 25, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 38. In certain embodiments, the antigen-binding site comprises the CDR-H1, CDR-H2, CDR-H3, CDR-L1, CDR-L2, and CDR-L3, determined under Kabat, AbM, Chothia, or any other CDR determination method known in the art, of the VH and VL sequences of SEQ ID NOs: 25 and 38, respectively. In certain embodiments, the VH comprises CDR-H1, CDR-H2, and CDR-H3, comprising the amino acid sequences of SEQ ID NOs: 3, 21, and 5, respectively.

In certain embodiments, an antigen-binding site described in the present application is derived from humanized RND-4. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 27, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 38. In certain embodiments, the antigen-binding site comprises the CDR-H1, CDR-H2, CDR-H3, CDR-L1, CDR-L2, and CDR-L3, determined under Kabat, AbM, Chothia, or any other CDR determination method known in the art, of the VH and VL sequences of SEQ ID NOs: 27 and 38, respectively. In certain embodiments, the VH comprises CDR-H1, CDR-H2, and CDR-H3, comprising the amino acid sequences of SEQ ID NOs: 3, 4, and 5, respectively. In certain embodiments, the VL comprises CDR-L1, CDR-L2, and CDR-L3 comprising the amino acid sequences of SEQ ID NOs: 32, 12, and 13, respectively. In certain embodiments, the antigen-binding site comprises (a) a VH that comprises CDR1, CDR2, and CDR3 comprising the amino acid sequences of SEQ ID NOs: 3, 4, and 5, respectively; and (b) a VL that comprises CDR1, CDR2, and CDR3 comprising the amino acid sequences of SEQ ID NOs: 32, 12, or 13, respectively.

In certain embodiments, an antigen-binding site described in the present application is derived from humanized RND-5. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 29, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 38. In certain embodiments, the antigen-binding site comprises the CDR-H1, CDR-H2, CDR-H3, CDR-L1, CDR-L2, and CDR-L3, determined under Kabat, AbM, Chothia, or any other CDR determination method known in the art, of the VH and VL sequences of SEQ ID NOs: 29 and 38, respectively.

In certain embodiments, an antigen-binding site described in the present application is derived from JML-1. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 121, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 126. In certain embodiments, the antigen-binding site comprises the CDR-H1, CDR-H2, CDR-H3, CDR-L1, CDR-L2, and CDR-L3, determined under Kabat, AbM, Chothia, or any other CDR determination method known in the art, of the VH and VL sequences of SEQ ID NOs: 121 and 126, respectively. In certain embodiments, the VH comprises CDR-H1, CDR-H2, and CDR-H3, comprising the amino acid sequences of SEQ ID NOs: 122, 123, and 124, respectively. In certain embodiments, the VL comprises CDR-L1, CDR-L2, and CDR-L3 comprising the amino acid sequences of SEQ ID NOs: 127, 128, and 129, respectively. In certain embodiments, the antigen-binding site comprises (a) a VH that comprises CDR1, CDR2, and CDR3 comprising the amino acid sequences of SEQ ID NOs: 122, 123, and 124, respectively; and (b) a VL that comprises CDR1, CDR2, and CDR3 comprising the amino acid sequences of SEQ ID NOs: 127, 128, and 129, respectively.

In certain embodiments, an antigen-binding site described in the present application is derived from murine M2. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 132, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 136. In certain embodiments, the antigen-binding site comprises the CDR-H1, CDR-H2, CDR-H3, CDR-L1, CDR-L2, and CDR-L3, determined under Kabat, AbM, Chothia, or any other CDR determination method known in the art, of the VH and VL sequences of SEQ ID NOs: 132 and 136, respectively. In certain embodiments, the VH comprises CDR-H1, CDR-H2, and CDR-H3, comprising the amino acid sequences of SEQ ID NOs: 133, 134, and 135, respectively. In certain embodiments, the VL comprises CDR-L1, CDR-L2, and CDR-L3 comprising the amino acid sequences of SEQ ID NOs: 137, 138, and 139, respectively. In certain embodiments, the antigen-binding site comprises (a) a VH that comprises CDR1, CDR2, and CDR3 comprising the amino acid sequences of SEQ ID NOs: 133, 134, and 135, respectively; and (b) a VL that comprises CDR1, CDR2, and CDR3 comprising the amino acid sequences of SEQ ID NOs: 137, 138, and 139, respectively.

In certain embodiments, an antigen-binding site described in the present application is derived from murine M7. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 140, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 144. In certain embodiments, the antigen-binding site comprises the CDR-H1, CDR-H2, CDR-H3, CDR-L1, CDR-L2, and CDR-L3, determined under Kabat, AbM, Chothia, or any other CDR determination method known in the art, of the VH and VL sequences of SEQ ID NOs: 140 and 144, respectively. In certain embodiments, the VH comprises CDR-H1, CDR-H2, and CDR-H3, comprising the amino acid sequences of SEQ ID NOs: 141, 142, and 143, respectively. In certain embodiments, the VL comprises CDR-L1, CDR-L2, and CDR-L3 comprising the amino acid sequences of SEQ ID NOs: 145, 146, and 147, respectively. In certain embodiments, the antigen-binding site comprises (a) a VH that comprises CDR1, CDR2, and CDR3 comprising the amino acid sequences of SEQ ID NOs: 141, 142, and 143, respectively; and (b) a VL that comprises CDR1, CDR2, and CDR3 comprising the amino acid sequences of SEQ ID NOs: 145, 146, and 147, respectively.

In certain embodiments, an antigen-binding site described in the present application is derived from murine M11. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 148, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 152. In certain embodiments, the antigen-binding site comprises the CDR-H1, CDR-H2, CDR-H3, CDR-L1, CDR-L2, and CDR-L3, determined under Kabat, AbM, Chothia, or any other CDR determination method known in the art, of the VH and VL sequences of SEQ ID NOs: 148 and 152, respectively. In certain embodiments, the VH comprises CDR-H1, CDR-H2, and CDR-H3, comprising the amino acid sequences of SEQ ID NOs: 149, 150, and 151, respectively. In certain embodiments, the VL comprises CDR-L1, CDR-L2, and CDR-L3 comprising the amino acid sequences of SEQ ID NOs: 153, 154, and 155, respectively. In certain embodiments, the antigen-binding site comprises (a) a VH that comprises CDR1, CDR2, and CDR3 comprising the amino acid sequences of SEQ ID NOs: 149, 150, and 151, respectively; and (b) a VL that comprises CDR1, CDR2, and CDR3 comprising the amino acid sequences of SEQ ID NOs: 153, 154, and 155, respectively.

In certain embodiments, an antigen-binding site described in the present application is derived from humanized M11-1. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 159, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 164. In certain embodiments, the antigen-binding site comprises the CDR-H1, CDR-H2, CDR-H3, CDR-L1, CDR-L2, and CDR-L3, determined under Kabat, AbM, Chothia, or any other CDR determination method known in the art, of the VH and VL sequences of SEQ ID NOs: 159 and 164, respectively. In certain embodiments, the VH comprises CDR-H1, CDR-H2, and CDR-H3, comprising the amino acid sequences of SEQ ID NOs: 149, 160, and 151, respectively. In certain embodiments, the VL comprises CDR-L1, CDR-L2, and CDR-L3 comprising the amino acid sequences of SEQ ID NOs: 165, 154, and 155, respectively. In certain embodiments, the antigen-binding site comprises (a) a VH that comprises CDR1, CDR2, and CDR3 comprising the amino acid sequences of SEQ ID NOs: 149, 160, and 151, respectively; and (b) a VL that comprises CDR1, CDR2, and CDR3 comprising the amino acid sequences of SEQ ID NOs: 165, 154, and 155, respectively.

In certain embodiments, an antigen-binding site described in the present application is derived from humanized M11-2. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 162, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 167. In certain embodiments, the antigen-binding site comprises the CDR-H1, CDR-H2, CDR-H3, CDR-L1, CDR-L2, and CDR-L3, determined under Kabat, AbM, Chothia, or any other CDR determination method known in the art, of the VH and VL sequences of SEQ ID NOs: 162 and 167, respectively.

In certain embodiments, an antigen-binding site described in the present application is derived from humanized M11-3. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO: 162, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 169. In certain embodiments, the antigen-binding site comprises the CDR-H1, CDR-H2, CDR-H3, CDR-L1, CDR-L2, and CDR-L3, determined under Kabat, AbM, Chothia, or any other CDR determination method known in the art, of the VH and VL sequences of SEQ ID NOs: 162 and 169, respectively.

In certain embodiments, antigen-binding sites disclosed herein bind human siglec-6 at a K_{D} value less than or equal to (affinity greater than or equal to) 900 nM, 800 nM, 700 nM, 600 nM, 500 nM, 400 nM, 300 nM, 200 nM, 100 nM, 90 nM, 80 nM, 70 nM, 60 nM, 50 nM, 45 nM, 40 nM, 35 nM, 30 nM, 25 nM, 20 nM, 15 nM, 10 nM, 9 nM, 8 nM, 7 nM, 6 nM, 5 nM, 4 nM, 3 nM, 2 nM, 1 nM, 0.5 nM, 0.4 nM, 0.3 nM, 0.2 nM, 0.1 nM, 90 pM, 80 pM, 70 pM, 60 pM, 50 pM, 40 pM, 30 pM, 20 pM, 10 pM, 9 pM, 8 pM, 7 pM, 6 pM, 5 pM, 4 pM, 3 pM, 2 pM, 1 pM, 0.9 pM, 0.8 pM, 0.7 pM, 0.6 pM, 0.5 pM, 0.4 pM, 0.3 pM, 0.2 pM, 0.1 pM, 90 fM, 80 fM, 70 fM, 60 fM, 50 fM, 40 fM, 30 fM, 20 fM, 10 fM, 9 fM, 8 fM, 7 fM, 6 fM, 5 fM, 4 fM, 3 fM, 2 fM, or 1 M. In certain embodiments, antigen-binding sites disclosed herein bind human siglec-6 at a K_{D} value less than or equal to (affinity greater than or equal to) 10 nM. In certain embodiments, antigen-binding sites disclosed herein bind human siglec-6 at a K_{D} value less than or equal to (affinity greater than or equal to) 0.1 nM, 0.2 nM, 0.3 nM, 0.4 nM, 0.5 nM, 0.6 nM, 0.7 nM, 0.8 nM, 0.9 nM, 1.0 nM, 1.1 nM, 1.2 nM, 1.3 nM, 1.4 nM, 1.5 nM, 1.6 nM, 1.7 nM, 1.8 nM, 1.9 nM, 2.0 nM, 2.1 nM, 2.2 nM, 2.3 nM, 2.4 nM, 2.5 nM, 2.6 nM, 2.7 nM, 2.8 nM, 2.9 nM, 3.0 nM, 3.1 nM, 3.2 nM, 3.3 nM, 3.4 nM, 3.5 nM, 3.6 nM, 3.7 nM, 3.8 nM, 3.9 nM, 4.0 nM, 4.1 nM, 4.2 nM, 4.3 nM, 4.4 nM, 4.5 nM, 4.6 nM, 4.7 nM, 4.8 nM, 4.9 nM, or 5.0 nM. In certain embodiments, the antigen binding site of the present application binds human siglec-6 at a K_{D} value in the range femtomolar to nanomolar. In certain embodiments, the antigen binding site of the present application binds human siglec-6 at a K_{D} value in the range of about 1 fM to about 900 nM, 1 fM to about 500 nM, 1 fM to about 100 nM, 1 fM to about 50 nM, 1 fM to about 10 nM, 1 fM to about 5 nM, 1 fM to about 1 nM, 1 fM to about 500 pM, 1 fM to about 100 pM, 1 fM to about 50 pM, 1 fM to about 10 pM, 1 fM to about 5 pM, 1 fM to about 1 pM, 1 fM to about 500 fM, 1 fM to about 100 fM, 1 fM to about 50 fM, 1 fM to about 10 fM, 1 fM to about 5 fM, 5 fM to about 900 nM, 5 fM to about 500 nM, 5 fM to about 100 nM, 5 fM to about 50 nM, 5 fM to about 10 nM, 5 fM to about 5 nM, 5 fM to about 1 nM, 5 fM to about 500 pM, 5 fM to about 100 pM, 5 fM to about 50 pM, 5 fM to about 10 pM, 5 fM to about 5 pM, 5 fM to about 1 pM, 5 fM to about 500 fM, 5 fM to about 100 fM, 5 fM to about 50 fM, 5 fM to about 10 fM, 10 fM to about 900 nM, 10 fM to about 500 nM, 10 fM to about 100 nM, 10 fM to about 50 nM, 10 fM to about 10 nM, 10 fM to about 5 nM, 10 fM to about 1 nM, 10 fM to about 500 pM, 10 fM to about 100 pM, 10 fM to about 50 pM, 10 fM to about 10 pM, 10 fM to about 5 pM, 10 fM to about 1 pM, 10 fM to about 500 fM, 10 fM to about 100 fM, 10 fM to about 50 fM, 50 fM to about 900 nM, 50 fM to about 500 nM, 50 fM to about 100 nM, 50 fM to about 50 nM, 50 fM to about 10 nM, 50 fM to about 5 nM, 50 fM to about 1 nM, 50 fM to about 500 pM, 50 fM to about 100 pM, 50 fM to about 50 pM, 50 fM to about 10 pM, 50 fM to about 5 pM, 50 fM to about 1 pM, 50 fM to about 500 fM, 50 fM to about 100 fM, 100 fM to about 900 nM, 100 fM to about 500 nM, 100 fM to about 100 nM, 100 fM to about 50 nM, 100 fM to about 10 nM, 100 fM to about 5 nM, 100 fM to about 1 nM, 100 fM to about 500 pM, 100 fM to about 100 pM, 100 fM to about 50 pM, 100 fM to about 10 pM, 100 fM to about 5 pM, 100 fM to about 1 pM, 100 fM to about 500 fM, 500 fM to about 900 nM, 500 fM to about 500 nM, 500 fM to about 100 nM, 500 fM to about 50 nM, 500 fM to about 10 nM, 500 fM to about 5 nM, 500 fM to about 1 nM, 500 fM to about 500 pM, 500 fM to about 100 pM, 500 fM to about 50 pM, 500 fM to about 10 pM, 500 fM to about 5 pM, 500 fM to about 1 pM, 1 pM to about 900 nM, 1 pM to about 500 nM, 1 pM to about 100 nM, 1 pM to about 50 nM, 1 pM to about 10 nM, 1 pM to about 5 nM, 1 pM to about 1 nM, 1 pM to about 500 pM, 1 pM to about 100 pM, 1 pM to about 50 pM, 1 pM to about 10 pM, 1 pM to about 5 pM, 5 pM to about 10 nM, 5 pM to about 5 nM, 5 pM to about 1 nM, 5 pM to about 500 pM, 5 pM to about 100 pM, 5 pM to about 50 pM, 5 pM to about 10 pM, 10 pM to about 10 nM, 10 pM to about 5 nM, 10 pM to about 1 nM, 10 pM to about 500 pM, 10 pM to about 100 pM, 10 pM to about 50 pM, 50 pM to about 10 nM, 50 pM to about 5 nM, 50 pM to about 1 nM, 50 pM to about 500 pM, 50 pM to about 100 pM, 100 pM to about 10 nM, 100 pM to about 5 nM, 100 pM to about 1 nM, 100 pM to about 500 pM, 500 pM to about 10 nM, 500 pM to about 5 nM, 500 pM to about 1 nM, 1 nM to about 10 nM, 1 nM to about 5 nM, or 5 nM to about 10 nM. In some embodiments, K_{D} values are measured using standard binding assays, for example, bio-layer interferometry.

In some embodiments, antigen-binding sites disclosed herein bind cynomolgus siglec-6 at a comparable affinity (K_{D} value) to that of binding human siglec-6. In some embodiments, the affinity of the antigen-binding site to cynomolgus siglec-6 relative to its affinity to human siglec-6, as measured in the same assay, is within a 2-fold, 3-fold, 5-fold, or 10-fold difference (either with greater affinity to human siglec-6 or with greater affinity to cynomolgus siglec-6).

In some embodiments, antigen-binding sites disclosed herein bind siglec-6 expressed on cell surface. In some embodiments, antigen-binding sites disclosed herein bind mast cells. In some embodiments, antigen-binding sites disclosed herein bind mast cells referentially to other immune cells (e.g., T cells, dendritic cells, macrophages, neutrophils, or basophils).

In some embodiments, antigen-binding sites disclosed herein does not significantly bind other siglec-6 family members. In some embodiments, antigen-binding sites disclosed herein does not significantly bind siglec 1-5, 7-12, 14, or 15.

In some embodiments, antigen-binding sites disclosed herein do not induce mass cell degranulation.

### Molecules Containing Antigen-Binding Sites

Also provided herein are molecules containing disclosed antigen-binding sites. Such molecules may be, but are not limited to, antibodies or antigen-binding fragments thereof, antibody fragments, nanobodies, antibody mimetics, etc.

In some embodiments, provided are antibodies or antigen-binding fragments thereof containing disclosed antigen-binding sites. In some embodiments, the antibodies or antigen-binding fragments thereof are multispecific, e.g., bispecific. In some embodiments, the antibodies are monoclonal antibodies. In some embodiments, the antibodies are humanized antibodies. In some embodiments, the antibodies are human antibodies. In some embodiments, the antibodies are non-fucosylated (afucosylated). In some embodiments, the antibodies are at most about 90%, at most about 80%, at most about 70%, at most about 60%, at most about 50%, at most about 40%, at most about 30%, at most about 25%, at most about 20%, at most about 15%, at most about 10%, at most about 5%, at most about 4%, at most about 3%, at most about 2%, at most about 1% fucosylated. In some embodiments, the antibodies are afucosylated. In some embodiments, the antibodies are at least about 30%, at least about 40%, at least about 50%, at least about 60%, at least about 70%, at least about 75%, at least about 80%, at least about 85%, at least about 90%, at least about 95%, at least about 96%, at least about 97%, at least about 98%, or at least about 99% afucosylated.

In some embodiments, the antibodies or antigen-binding fragments thereof are capable of binding c-Kit. In some embodiments, the antibodies or antigen-binding fragments thereof are capable of binding siglec-6. In some embodiments, the antibodies or antigen-binding fragments thereof are capable of binding c-Kit and siglec-6.

In some embodiments, provided are antibody fragments thereof containing disclosed antigen-binding sites. For example, the fragments may be, but not limited to, an Fab, an scFab (single-chain Fab), an F(ab′)₂, a Fab′, a single-chain Fv (scFv), an Fv fragment, or a diabody (a dimer of scFv). In some embodiments, the antibody fragments are multispecific, e.g., bispecific. In some embodiments, the antibody fragments are capable of binding c-Kit. In some embodiments, the antibody fragments are capable of binding siglec-6. In some embodiments, the antibody fragments are capable of binding c-Kit and siglec-6.

In some embodiments, provided are antibody mimetics containing disclosed antigen-binding sites. For example, the fragments may be, but not limited to, an Affibody, an Affilin, an Affimer, an afftin, an Alphabody, an Anticalin, an avimer, a Centyrin, a DARPin, a Fynomer, a monobody, or a nanoCLAMP. In some embodiments, the antibody mimetics are multispecific, e.g., bispecific. In some embodiments, the antibody mimetics are capable of binding c-Kit. In some embodiments, the antibody mimetics are capable of binding siglec-6. In some embodiments, the antibody mimetics are capable of binding c-Kit and siglec-6.

In some embodiments, provided are bispecific molecules comprising two antigen-binding sites disclosed herein.

In some embodiments, the bispecific molecule comprises an antigen-binding site capable of binding c-Kit described herein. In some embodiments, the bispecific molecule comprises an antigen-binding site capable of binding siglec-6 described herein. In some embodiments, the bispecific molecule comprises an antigen-binding site capable of binding c-Kit described herein and an antigen-binding site capable of binding siglec-6 described herein.

In some embodiments, provided are bispecific molecules comprising: (i) an antigen-binding site capable of binding siglec-6 described herein; and (ii) an antigen-binding site capable of binding c-Kit described herein. In some embodiments, the bispecific molecule comprises a first antigen-binding site comprising the VH region and the VL region of an antigen-binding site capable of binding siglec-6 described herein and a second antigen-binding site comprising the VH region and the VL region of an antigen-binding site capable of binding c-Kit described herein. In some embodiments, the first antigen-binding site (that is capable of binding siglec-6) comprises a VH region and a VL region which is, or which is derived from, the VH/VL region of murine RND, humanized RND, or JML-1, as described herein; and the second antigen-binding domain (that is capable of binding c-Kit) comprises a VH region and a VL region which is, or which is derived from, the VH/VL region of muthe SR1 or humanized SR1, as described herein.

In some embodiments, the bispecific molecule is capable of binding a cell expressing siglect-6. In some embodiments, the bispecific molecule is capable of binding a cell expressing c-Kit. In some embodiments, the bispecific molecule is capable of binding a mast cell. In some embodiments, the bispecific molecule binds a mast cell preferentially than another immune cell (e.g., T cell, dendritic cell, macrophage, neutrophil, or basophil). In some embodiments, the bispecific molecule has a comparable or lower internalization rate compared to a reference antigen-binding site capable of binding siglect-6 (e.g., an antibody comprising a VH and a VL of SEQ ID NOs: 2 and 10, respectively). In some embodiments, the bispecific molecule has a comparable or lower toxicity compared to a reference antigen-binding site capable of binding c-Kit (e.g., an antibody comprising a VH and a VL of SEQ ID NOs: 40 and 45, respectively).

Various formats and uses of bispecific molecules are known in the art. Bispecific molecules according to the present invention are not limited to any particular bispecific format or method of producing it.

In some embodiments, the bispecific molecule may be a bispecific antibody or a fragment or derivative thereof including, but not limited to, (i) a single antibody that has two arms, each comprising a different antigen-binding site; (ii) a bispecific scFv, e.g., via two scFvs linked by a peptide linker; (iii) a dual-variable-domain antibody (DVD-Ig), where each light chain and heavy chain contains two variable domains in tandem through a short peptide linkage; (iv) a bispecific (Fab′)₂ fragment; (v) a diabody; (vi) a tandem diabody (TandAb), a fusion of two diabodies; and (vii) a “dock-and-lock (DNL)-Fab3, a trivalent bispecific binding protein consisting of two identical Fab fragments linked to a different Fab fragment; (viii) IgG-like molecules with engineered Fc to force heterodimerization; (ix) recombinant IgG-like dual targeting molecules, wherein the two sides of the molecule each contain the Fab fragment or part of the Fab fragment of at least two different antibodies; (x) IgG fusion molecules, wherein full length IgG antibodies are fused to extra Fab fragment or parts of Fab fragment; (xi) Fc fusion molecules; (v) Fab fusion molecules; and (vi) scFv- and diabody-based and heavy chain antibodies (e.g., domain antibodies, nanobodies) wherein different scFvs, diabodies, or heavy-chain antibodies (e.g., domain antibodies, nanobodies) are fused to each other or to another protein or carrier molecule fused to heavy-chain constant-domains, Fc-regions or parts thereof.

Examples of IgG-like molecules with engineered Fc include, but are not limited, to the triomab, knobs-into-holes (kih) molecules (e.g., kih IgG with common light chain), CrossMAbs, orthoFab IgG molecules electrostatically-matched molecules, the LUZ-Y molecules, DIG-body and PIG-body molecules, the Strand Exchange Engineered Domain body (SEEDbody) molecules, the Biclonics molecules, FcAAdp molecules, bispecific IgG1 and IgG2 molecules, Azymetric scaffold molecules, and the DuoBody molecules. Examples of recombinant IgG-like dual targeting molecules include, but are not limited, to Dual Targeting (DT)-Ig molecules, two-in-one antibody, mAb², and Zybody. Examples of IgG fusion molecules include, but are not limited to, DVD-Ig molecules and IgG-scFv. Examples of Fc fusion molecules include, but are not limited to, scFv/Fc fusions, SCORPION molecules, and Fc-DART molecules. Examples of Fab fusion bispecific antibodies include, but are not limited to, F(ab)₂ molecules, DNL molecules, and Fab-Fv molecules. Examples of scFv- and diabody-based and domain antibodies include, but are not limited, to bispecific T cell engager (BiTE) molecules, tandem diabody molecules (TandAb), dual-affinity retargeting technology (DART) molecules, single-chain diabody molecules, TCR-like antibodies (AIT, ReceptorLogics), human serum albumin scfv fusion, COMBODY molecules, dual targeting nanobodies, and dual targeting heavy chain only domain antibodies.

In certain embodiments, disclosed bispecific molecules may be fusion proteins containing one or more antibody mimetics. In certain embodiments, disclosed bispecific molecules may be fusion proteins containing one or more antibodies or antigen-binding fragments thereof. In some embodiments, disclosed bispecific molecule may be fusion proteins containing one or more antibody mimetics and one or more antibodies or antigen-binding fragments thereof.

In some embodiments, molecules containing disclosed antigen-binding sites further comprise antibody constant regions or fragments or variants thereof. In some embodiments, the antibody constant region may be, e.g., a heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In certain embodiments, the antibody constant region or fragment or variant thereof has an amino acid sequence at least 90% (e.g., 90%, 91%, 92%, 93%, 94%, 95%, 96%, 97%, 98%, 99%, or 100%) identical to a heavy chain constant region of, e.g., the heavy chain constant region of IgG1, IgG2, IgG3, IgG4, IgM, IgA1, IgA2, IgD, or IgE, preferably, e.g., the heavy chain constant region of human IgG1, IgG2, IgG3, or IgG4. In certain embodiments, the antibody constant region may be a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment, the antibody has effector function and/or can fix complement. In other embodiments, the antibody does not recruit effector cells or fix complement. In another embodiment, the antibody has reduced or no ability to bind an Fc receptor. For example, it is an isotype or subtype, fragment or other mutant, which does not support binding to an Fc receptor, e.g., it has a mutagenized or deleted Fc receptor binding region.

In some embodiments, the constant region comprises one or more mutation(s) that reduce effector function. For example, in certain embodiments, the constant region comprises a heavy chain constant region of IgG1, comprising one or more of the following mutations, numbering based on EU index, (a) L234A and/or L235A; (b) A327G, A330S, and/or P331S; (c) E233P, L234V, L235A, and/or G236del; (d) E233P, L234V, and/or L235A; (e) E233P, L234V, L235A, G236del, A327G, A330S, and/or P331S; (f) E233P, L234V, L235A, A327G, A330S, and/or P331S; (g) N297A; (h) N297G; (i) N297Q; (j) L242C, N297C, and/or K334C; (k) A287C, N297G, and/or L306C; (l) R292C, N297G, and/or V302C; (m) N297G, V323C, and/or I332C; (n) V259C, N297G, and/or L306C; (o) L234F, L235Q, K322Q, M252Y, S254T, and/or T256E; (p) L234A, L235A, and/or P329G; or (q) L234A, L235Q, and K322Q. In certain embodiments, the constant region comprises a heavy chain constant region of IgG2, comprising one or more of the following mutations, numbering based on EU index, (a) A330S and/or P331S; (b) V234A, G237A, P238S, H268A, V309L, A330S, and/or P331S; or (c) V234A, G237A, H268Q, V309L, A330S, P331S, C232S, C233S, S267E, L328F, M252Y, S254T, and/or T256E. In certain embodiments, the constant region comprises a heavy chain constant region of IgG4, comprising one or more of the following mutations, numbering based on EU index, (a) E233P, F234V, L235A, and/or G236del; (b) E233P, F234V, and/or L235A; (c) S228P and/or L235E; or (d) S228P and/or L235A.

In some embodiments, the constant region comprises one or more mutation(s) that enhance effector function. For example, in certain embodiments, the constant region comprises a heavy chain constant region of IgG1, comprising one or more of the following mutations, numbering based on EU index, (a) F243L, R292P, Y300L, V305I, and/or P396L; (b) S239D and/or I332E; (c) S239D, I332E, and/or A330L; (d) S298A, E333A, and/or K334A; (e) G236A, S239D, and/or I332E; (f) K326W and/or E333S; (g) S267E, H268F, and/or S324T; or (h) E345R, E430G, and/or S440Y.

In some embodiments, a disclosed antigen-binding site is linked to an antibody constant region or fragment or variant thereof. In certain embodiments, the antigen-binding site is linked to an IgG constant region including hinge, CH₂ and CH₃ domains with or without a CH₁ domain.

In some embodiments, molecules containing disclosed antigen-binding sites further comprises a Fc region that is non-fucosylated. In some embodiments, the Fc region is non-fucosylated IgG1 Fc region.

### Amino Acid Sequence Modifications

Amino acid sequence modification(s) of the antigen-binding sites and molecules containing antigen-binding sites (e.g., antibodies or antigen-binding fragments thereof) disclosed herein are contemplated. For example, it may be desirable to improve the binding affinity and/or other biological properties of the antibodies or antigen-binding fragments. Amino acid sequence variants can be prepared, e.g., by introducing appropriate nucleotide changes into a nucleic acid sequence encoding the antigen-binding site or molecule containing antigen-binding site, or by peptide synthesis. Such modifications can include, for example, deletions from, and/or insertions into and/or substitutions of, residues within the amino acid sequences. Any combination of deletion, insertion, and substitution can be made, provided that the antigen-binding site or molecule containing antigen-binding site retains the desired characteristics and/or functions. In some embodiments, amino acid changes are introduced to alter post-translational processes, such as changing the number or position of glycosylation sites.

A useful method for identification of certain residues or regions that are preferred locations for mutagenesis is called “alanine scanning mutagenesis.” In this method, a residue or group of target residues are identified (e.g., charged residues such as Arg, Asp, His, Lys, and Glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis may be conducted at the target codon or region, and the expressed variants may be screened for a desired activity.

Examples of amino acid sequence insertions include, but are not limited to, amino- and/or carboxyl-terminal fusions ranging in length from one residue to polypeptides containing a hundred or more residues, as well as intrasequence insertions of single or multiple amino acid residues. An example of a terminal insertion includes, but are not limited to, N-terminal methionyl residues.

In some embodiments, the antigen-binding site or molecule containing antigen-binding site is fused at one terminus to another polypeptide, e.g., a cytotoxic polypeptide, an enzyme, or a polypeptide which increases the serum half-life of the antibody or antigen-binding fragment.

Another type of variant is an amino acid substitution variant. These variants have at least one amino acid residue in the amino acid sequence of the molecule replaced by a different residue. Sites of greatest interest for substitutional mutagenesis are typically the hypervariable regions, but framework region alterations are also contemplated. Examples of conservative substitutions are shown in Table 1 under the heading of “preferred substitutions.” More substantial changes, under the heading “exemplary substitutions” in Table 1, or as further described below in reference to amino acid classes, may be introduced and the resulting antibodies or antigen-binding fragments screened.

TABLE 1
Exemplary Amino acid substitutions

| Original | Exemplary | Preferred |
|---|---|---|
| Residue | Substitutions | Substitutions |
| Ala (A) | Val, Leu, Ile | Val |
| Arg (R) | Lys, Gln, Asn | Lys |
| Asn (N) | Gln, His, Asp, Lys, Arg | Gln |
| Asp (D) | Glu. Asn | Glu |
| Cys (C) | Ser, Ala | Ser |
| Gln (Q) | Asn, Glu | Asn |
| Glu (E) | Asp, Gln | Asp |
| Gly (G) | Ala | Ala |
| His (H) | Asn, Gln, Lys, Arg | Arg |
| Ile (I) | Leu, Val, Met, Ala, Phe, Norleucine | Leu |
| Leu (L) | Norleucine, Ile, Val, Met, Ala, Phe | Ile |
| Lys (K) | Arg, Gln, Asn | Arg |
| Met (M) | Leu Phe, Ile | Leu |
| Phe (F) | Leu, Val, Ile, Ala, Tyr | Tyr |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala, Norleucine | Leu |

Substantial modifications in the biological properties of the antibody may be accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are typically divided into groups based on common side-chain properties:
(1) hydrophobic: Norleucine, Met, Ala, Val, Leu, Ile;
(2) neutral hydrophilic: Cys, Ser, Thr;
(3) acidic: Asp, Glu;
(4) basic: Asn, Gln, His, Lys, Arg;
(5) residues that influence chain orientation: Gly, Pro; and
(6) aromatic: Trp, Tyr, Phe.

Non-conservative substitutions can entail exchanging a member of one of these classes for another class.

Additionally or alternatively, cysteine residues not involved in maintaining the proper conformation of the antibody or antigen-binding fragment may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

In some embodiments, a substitutional variant comprises a substitution within one or more hypervariable region residues of a parent antibody (e.g., a human antibody). Generally, the resulting variant(s) having improved biological properties relative to the parent antibody from which they are generated are selected for further development.

A method for generating such substitutional variants involves affinity maturation using phage display. In an example of such a method, several hypervariable region sites (e.g., 6-7 sites) are mutated to generate all possible amino substitutions at each site. Antibody variants thus generated are displayed in a monovalent fashion, e.g., from filamentous phage particles as fusions to the gene III product of M13 packaged within each particle. The phage-displayed variants are then screened for their biological activity (e.g., binding affinity).

To identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody or antigen-binding fragment and the antigen. Such contact residues and neighboring residues are candidates for substitution according to the techniques elaborated herein. Once such variants are generated, the panel of variants is subjected to screening, and antibodies with superior properties in one or more relevant assays may be selected for further development.

In some embodiments, the original glycosylation pattern of a parent antibody is altered. Such alteration(s) may comprise deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are recognition sequences for enzymatic attachment of the carbohydrate moiety to an asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody may be accomplished by altering the antibody or antigen-binding fragment's amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of antibodies or antigen-binding fragments may be prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody or antigen-binding fragment thereof.

In some embodiments, a modification that increases the serum half-life is used. For example, a salvage receptor binding epitope can be incorporated into a molecule containing disclosed antigen-binding site as described, e.g., in U.S. Pat. No. 5,739,277. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

### Methods of Making Antigen-Binding Sites and Molecules Containing Antigen-Binding Sites

The proteins herein above can be made using recombinant DNA technology well known to a skilled person in the art.

For example, one or more nucleic acid sequences encoding a protein containing the disclosed antigen-binding site can be cloned into one or more expression vectors; the expression vectors can be stably transfected into host cells capable of expressing the gene(s). After transfection, single clones can be isolated for cell bank generation using methods known in the art, such as limited dilution, ELISA, FACS, microscopy, or Clonepix. Clones can be cultured under conditions suitable for bio-reactor scale-up and maintained expression of a protein comprising an antigen-binding site disclosed herein. The protein can be isolated and purified using methods known in the art, such as centrifugation, depth filtration, cell lysis, homogenization, freeze-thawing, affinity purification, gel filtration, ion exchange chromatography, hydrophobic interaction exchange chromatography, and mixed-mode chromatography.

Accordingly, also provided herein are isolated nucleic acids encoding antigen-binding sites and molecules (e.g., proteins) containing antigen-binding sites, vectors and host cells comprising the nucleic acid, and recombinant techniques for the production.

In certain embodiments, provided are one or more isolated nucleic acids comprising sequences encoding an immunoglobulin heavy chain and/or immunoglobulin light chain variable region of any antibody disclosed herein, and one or more expression vectors that express the immunoglobulin heavy chain and/or immunoglobulin light chain variable region of any antibody disclosed herein. Also provided are host cells comprising one or more of the foregoing expression vectors and/or isolated nucleic acids.

In some embodiments, antigen-binding sites and molecules (e.g., proteins) containing antigen-binding sites of the present disclosure may be fused to another agent, e.g., another therapeutic agent. Construction of fusion proteins is within ordinary skill in the art.

### Monoclonal Antibodies

Monoclonal antibodies may be made using the hybridoma method first described by, or may be made by recombinant DNA methods (U.S. Pat. No. 4,816,567).

In the hybridoma method, a mouse or other appropriate host animal, such as a hamster, is immunized to elicit lymphocytes that produce or are capable of producing antibodies that will specifically bind to the protein used for immunization. Alternatively or additionally, lymphocytes may be immunized in vitro. After immunization, lymphocytes are isolated and then fused with a myeloma cell line using a suitable fusing agent, such as polyethylene glycol, to form a hybridoma cell.

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Examples of suitable fusion partner include, but are not limited to, myeloma cells are those that fuse efficiently, support stable high-level production of antibody by the selected antibody-producing cells, and are sensitive to a selective medium that selects against the unfused parental cells. Examples of suitable myeloma cell lines include, but are not limited to, are murine myeloma lines, such as those derived from MOPC-21 and MPC-11 mouse tumors available from the Salk Institute Cell Distribution Center, San Diego, Calif USA, and SP-2 and derivatives e.g., X63-Ag8-653 cells available from the American Type Culture Collection, Rockville, Md. USA. Human myeloma and mouse-human heteromyeloma cell lines also have been described for the production of human monoclonal antibodies.

According to the hybridoma method, culture medium in which hybridoma cells are growing is then assayed for production of monoclonal antibodies directed against the antigen. For example, the binding specificity of monoclonal antibodies produced by hybridoma cells may be determined by immunoprecipitation or by an in vitro binding assay, such as radioimmunoassay (RIA) or enzyme-linked immunosorbent assay (ELISA).

The binding affinity of the monoclonal antibody can, for example, be determined by a Scatchard analysis.

Once hybridoma cells that produce antibodies of the desired specificity, affinity, and/or activity are identified, clones may be subcloned, e.g., by limiting dilution procedures and grown by standard methods. Suitable culture media for this purpose include, for example, D-MEM or RPMI-1640 medium. In addition, the hybridoma cells may be grown in vivo as ascites tumors in an animal, e.g., by i.p. injection of the cells into mice.

Monoclonal antibodies secreted by the subclones are suitably separated from the culture medium, ascites fluid, or serum by conventional antibody purification procedures such as, for example, affinity chromatography (e.g., using protein A or protein G-Sepharose®) or ion-exchange chromatography, hydroxylapatite chromatography, gel electrophoresis, dialysis, etc.

DNA encoding the monoclonal antibodies can be readily isolated and sequenced using conventional procedures (e.g., by using oligonucleotide probes that are capable of binding specifically to genes encoding the heavy and light chains of murine antibodies). Hybridoma cells can serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells.

In certain embodiments, monoclonal antibodies or antibody fragments are isolated from antibody phage libraries. High affinity (nM range) human antibodies can be produced, e.g., by chain shuffling. Combinatorial infection and in vivo recombination may provide strategies for constructing very large phage libraries. These techniques are viable alternatives to traditional monoclonal antibody hybridoma techniques for isolation of monoclonal antibodies.

DNA that encodes the antibody may be modified to produce chimeric or fusion antibody polypeptides, for example, by substituting human heavy chain and light chain constant domain (CH and CL) sequences for the homologous murine sequences (see, e.g., U.S. Pat. No. 4,816,567), or by fusing the immunoglobulin coding sequence with all or part of the coding sequence for a non-immunoglobulin polypeptide (heterologous polypeptide).

### Human Antibodies and Phage Display Methodology

Human antibodies can be generated by methods known in the art, including methods described herein. For example, it is possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., U.S. Pat. Nos. 5,545,806, 5,569,825, 5,591,669; 5,545,807; and WO 97/17852.

Alternatively, phage display technology can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats. Several sources of V-gene segments can be used for phage display, e.g., from random combinatorial librarues of V genes such as libraries derived from the spleens of immunized mice. A repertoire of V genes from unimmunized human donors can be constructed and antibodies to a diverse array of antigens (including self-antigens) can be isolated essentially following methods described in the art. See, e.g., U.S. Pat. Nos. 5,565,332 and 5,573,905.

Human antibodies may also be generated by in vitro activated B cells (see, e.g., U.S. Pat. Nos. 5,567,610 and 5,229,275).

Competition assays for determining whether an antibody binds to the same epitope as, or competes for binding with a disclosed antibody are known in the art. Exemplary competition assays include immunoassays (e.g., ELISA assays, RIA assays), surface plasmon resonance (e.g., BIAcore analysis), bio-layer interferometry, and flow cytometry.

Typically, a competition assay involves the use of an antigen bound to a solid surface or expressed on a cell surface, a test antibody, and a reference antibody. The reference antibody is labeled and the test antibody is unlabeled. Competitive inhibition is measured by determining the amount of labeled reference antibody bound to the solid surface or cells in the presence of the test antibody. Usually the test antibody is present in excess (e.g., lx, 5×, 10×, 20× or 100×). Antibodies identified by competition assay (e.g., competing antibodies) include antibodies binding to the same epitope, or similar (e.g., overlapping) epitopes, as the reference antibody, and antibodies binding to an adjacent epitope sufficiently proximal to the epitope bound by the reference antibody for steric hindrance to occur.

A competition assay can be conducted in both directions to ensure that the presence of the label does not interfere or otherwise inhibit binding. For example, in the first direction the reference antibody is labeled and the test antibody is unlabeled, and in the second direction, the test antibody is labeled and the reference antibody is unlabeled.

A test antibody competes with the reference antibody for specific binding to the antigen if an excess of one antibody (e.g., 1×, 5×, 10×, 20× or 100×) inhibits binding of the other antibody, e.g., by at least 50%, 75%, 90%, 95% or 99% as measured in a competitive binding assay.

Two antibodies may be determined to bind to the same epitope if essentially all amino acid mutations in the antigen that reduce or eliminate binding of one antibody reduce or eliminate binding of the other. Two antibodies may be determined to bind to overlapping epitopes if only a subset of the amino acid mutations that reduce or eliminate binding of one antibody reduce or eliminate binding of the other.

The antibodies disclosed herein may be further optimized (e.g., affinity-matured) to improve biochemical characteristics including affinity and/or specificity, improve biophysical properties including aggregation, stability, precipitation and/or non-specific interactions, and/or to reduce immunogenicity. Affinity-maturation procedures are within ordinary skill in the art. For example, diversity can be introduced into an immunoglobulin heavy chain and/or an immunoglobulin light chain by DNA shuffling, chain shuffling, CDR shuffling, random mutagenesis and/or site-specific mutagenesis.

In certain embodiments, isolated human antibodies contain one or more somatic mutations. In these cases, antibodies can be modified to a human germline sequence to optimize the antibody (e.g., by a process referred to as germlining).

Generally, an optimized antibody has at least the same, or substantially the same, affinity for the antigen as the non-optimized (or parental) antibody from which it was derived. For example, in certain embodiments, an optimized antibody has a higher affinity for the antigen when compared to the parental antibody.

### Pharmaceutical Compositions

In certain embodiments, provided molecules containing disclosed antigen-binding sites are incorporated together with one or more pharmaceutically acceptable carriers into a pharmaceutical composition suitable for administration to a subject. As used herein, “pharmaceutically acceptable carrier” refers to any of a variety of solvents, dispersion media, coatings, antibacterial and antifungal agents, isotonic and absorption delaying agents, and the like that are physiologically compatible. Examples of pharmaceutically acceptable carriers include, but are not limited to, water, saline, phosphate buffered saline, dextrose, glycerol, ethanol and the like, as well as combinations thereof.

In some embodiments, pharmaceutical compositions comprise one or more tonicity agents or stabilizers. Non-limiting examples of such tonicity agents or stabilizers include sugars (e.g., sucrose), polyalcohols (e.g., mannitol or sorbitol), and sodium chloride.

In some embodiments, pharmaceutical compositions comprise one or more bulking agents and/or lyoprotectants (e.g., mannitol or glycine), buffers (e.g., phosphate, acetate, or histidine buffers), surfactants (e.g., polysorbates), antioxidants (e.g., methionine), and/or metal ions or chelating agents (e.g., ethylenediaminetetraacetic acid (EDTA)).

In some embodiments, pharmaceutical compositions comprise one or more auxiliary substances such as wetting or emulsifying agents, preservatives (e.g., benzyl alcohol) or buffers, which may enhance the shelf life and/or effectiveness of immunoconjugates disclosed herein.

Pharmaceutical compositions may be provided in any of a variety of forms. These include, for example, liquid, semi-solid and solid dosage forms, such as liquid solutions (e.g., injectable and infusible solutions), dispersions or suspensions, tablets, pills, powders, liposomes and suppositories. Suitability of certain forms may depend on the intended mode of administration and therapeutic application.

In some embodiments, pharmaceutical compositions are in the form of injectable or infusible solutions.

Pharmaceutical compositions are typically sterile and stable under conditions of manufacture, transport, and storage. Pharmaceutical compositions may be formulated as, for example, a solution, microemulsion, dispersion, liposome, or other ordered structure. In some embodiments, a pharmaceutical composition is formulated as a structure particularly suitable for high drug concentration. For example, sterile injectable solutions can be prepared by incorporating a therapeutic agent (e.g., immunoconjugate) in a desired amount in an appropriate solvent with one or a combination of ingredients enumerated herein, optionally followed by sterilization (e.g., filter sterilization). Generally, dispersions may be prepared by incorporating an immunoconjugate into a sterile vehicle that contains a basic dispersion medium and other ingredient(s) such as those additional ingredients mentioned herein. In the case of sterile powders for the preparation of sterile injectable solutions, examples of preparation methods include vacuum drying and freeze-drying to yield a powder of the immunoconjugate and any additional desired ingredient(s), e.g., from a previously sterile-filtered solution thereof.

Proper fluidity of a solution can be maintained, for example, by the use of a coating such as lecithin, by maintaining certain particle sizes (e.g., in the case of dispersions), and/or by using surfactants. Prolonged absorption of injectable compositions can be brought about, e.g., by including in the composition an agent that delays absorption (for example, monostearate salts and/or gelatin).

### Methods of Treatment

The present application provides methods for treating a disease or disorder using a molecule (e.g., protein, such as antibody or antigen-binding fragment thereof) or pharmaceutical composition described herein. Methods of treatment disclosed herein generally comprise a step of administering a therapeutically effective amount of a disclosed molecule containing antigen-binding site or a pharmaceutical composition thereof to a subject (e.g., a human subject) in need thereof.

In some embodiments, the disease or disorder is a mast cell associated disease or disorder. In some embodiments, the mast cell associated disease or disorder is related to undesirable mast cell activity, e.g., excessive proliferation of mast cells. Non-limiting examples of mast cell associated diseases or disorders include upper airway diseases such as allergic rhinitis and sinusitis, foreign body aspiration, glottic stenosis, tracheal stenosis, laryngotracheomalacia, vascular rings, chronic obstructive pulmonary disease (COPD), and congestive heart failure, eosinophilic bronchitis, polychondritis, sarcoidosis, papillomatosis, arthritis (e.g., rheumatoid arthritis), Wegener's granulomatosis, primary biliary cirrhosis, an allergy that is responsive to oral immunotherapy, secondary sclerosing cholangitis due to mast cell cholangiopathy, pruritus, itching caused by or associated with chronic kidney disease associated pruritis or primary biliary cirrohosis, bronchiolitis obliterans syndrome after lung transplantation or hematopoietic stem cell transplantation, inflammatory bowel syndrome, functional dyspepsia, bronchopulmonary dysplasia in infants, cerebral ischemia, seizures, pneumonia caused by influenza, sepsis, scleroderma, cluster headaches, cardiac fibrosis, primary sclerosing cholangitis (PSC), Morbihan disease, aspirin intolerant asthma, vulvodynia, chronic pelvic pain syndrome (prostatitis), and cancer (e.g., lung cancer).

In some embodiments, the disease or disorder is an autoimmune disease. In some embodiments, the disease or disorder is an allergy. In some embodiments, the disease or disorder is an inflammation. In some embodiments, the disease or disorder is pruritus. In some embodiments, the disease or disorder is a cancer.

Therapeutically effective amounts may be administered via a single dose or via multiple doses (e.g., at least two, at least three, at least four, at least five, at least six, at least seven, at least eight, at least nine, or at least ten doses). When administered via multiple doses, any of a variety of suitable therapeutic regimens may be used, including administration at regular intervals (e.g., once every other day, once every three days, once every four days, once every five days, thrice weekly, twice weekly, once a week, once every two weeks, once every three weeks, etc.).

The dosage regimen (e.g., amounts of each therapeutic, relative timing of therapies, etc.) that is effective in methods of treatment may depend on the severity of the disease or condition and the weight and general state of the subject. For example, the therapeutically effective amount of a particular composition comprising a therapeutic agent applied to mammals (e.g., humans) can be determined by the ordinarily-skilled artisan with consideration of individual differences in age, weight, and the condition of the mammal. Therapeutically effective and/or optimal amounts can also be determined empirically by those of skill in the art.

The molecule containing antigen-binding site or pharmaceutical composition thereof may be administered by any of a variety of suitable routes, including, but not limited to, systemic routes such as parenteral (e.g., intravenous or subcutaneous) or enteral routes.

### LIST OF EXEMPLARY EMBODIMENTS

The invention is further described by the following non-limiting exemplary embodiments:
Embodiment 1. An antigen-binding site that is capable of binding to FCER1A, FCER1B, and/or FCER1G.
Embodiment 2. The antigen-binding site of embodiment 1, wherein the antigen-binding site is capable of binding to FCER1A.
Embodiment 3. The antigen-binding site of embodiment 1, wherein the antigen-binding site is capable of binding to FCER1B.
Embodiment 4. The antigen-binding site of embodiment 1, wherein the antigen-binding site is capable of binding to FCER1G.
Embodiment 5. An antigen-binding site that is capable of binding to c-Kit.
Embodiment 6. An antigen-binding site that is capable of binding to siglec-6.
Embodiment 7. An antigen-binding site that is capable of binding to siglec-8.
Embodiment 8. The antigen-binding site of any one of embodiments 1-7, wherein the antigen-binding site is present as a single-chain fragment variable (scFv).
Embodiment 9. The antigen-binding site of any one of embodiments 1-7, wherein the antigen-binding site is present as an antibody mimetic (e.g., an Affibody, an Affilin, an Affimer, an afftin, an Alphabody, an Anticalin, an avimer, a Centyrin, a DARPin, a Fynomer, a monobody, or a nanoCLAMP).
Embodiment 10. The antigen-binding site of any one of embodiments 5 and 8-9, wherein the antigen-binding site comprises
(a) the complementarity-determining region 1 (CDR1), CDR2, and/or CDR3 of a heavy chain (HC) set forth in any one of SEQ ID NO: 39, 49, 51, 54, 56, or 58; and/or
(b) the CDR1, CDR2, and/or CDR3 of a light chain (LC) set forth in any one of SEQ ID NO: 44, 50, 86, or 88.
Embodiment 11. The antigen-binding site of any one of embodiments 5 and 8-10, wherein the antigen-binding site comprises
(a) a heavy chain variable domain (VH) comprising complementarity-determining regions:
   CDR-H1 comprising the amino acid sequence: SYNMH (SEQ ID NO: 41),
   CDR-H2 comprising the amino acid sequence: VIYSGNGDTSYNQKFKG (SEQ ID NO: 42) or IYSGNGDTSYNQKFKGK (SEQ ID NO: 33), and
   CDR-H3 comprising the amino acid sequence: ERDTRFGN (SEQ ID NO: 43); and/or
(b) a light chain variable domain (VL) comprising complementarity-determining regions:
   CDR-L1 comprising the amino acid sequence: RASESVDIYGNSFMH (SEQ ID NO: 46);
   CDR-L2 comprising the amino acid sequence: LASNLES (SEQ ID NO: 47); and
   CDR-L3 comprising the amino acid sequence: QQNNEDPYT (SEQ ID NO: 48).
Embodiment 12. The antigen-binding site of any one of embodiments 5 and 8-11, wherein the antigen-binding site comprises
(a) a VH comprising an amnio acid sequence having at least 85% identity to the VH of a HC set forth in SEQ ID NO: 39; and/or
   a VL comprising an amnio acid sequence having at least 85% identity to the VL of a LC set forth in SEQ ID NO: 44; or
(b) a VH comprising an amnio acid sequence having at least 85% identity to the VH of a HC set forth in SEQ ID NO: 49; and/or
   a VL comprising an amnio acid sequence having at least 85% identity to the VL of a LC set forth in SEQ ID NO: 50; or
(c) a VH comprising an amnio acid sequence having at least 85% identity to the VH of a HC set forth in any one of SEQ ID NOs: 51, 54, 56, and 58; and/or
   a VL comprising an amnio acid sequence having at least 85% identity to the VL of a LC set forth in SEQ ID NO: 86 or 88.
Embodiment 13. The antigen-binding site of any one of embodiments 5 and 8-12, wherein the antigen-binding site comprises
(a) a HC comprising CDR-H1, CDR-H2, and CDR-H3 of a HC set forth in SEQ ID NO: 39, and having at least 85% amino acid sequence identity to SEQ ID NO: 39; and/or a LC comprising CDR-L1, CDR-L2, and CDR-L3 of a LC set forth in SEQ ID NO: 44, and having at least 85% amino acid sequence identity to SEQ ID NO: 44; or
(b) a HC comprising CDR-H1, CDR-H2, and CDR-H3 of a HC set forth in SEQ ID NO: 49, and having at least 85% amino acid sequence identity to SEQ ID NO: 49; and/or a LC comprising CDR-L1, CDR-L2, and CDR-L3 of a LC set forth in SEQ ID NO: 50, and having at least 85% amino acid sequence identity to SEQ ID NO: 50; or
(c) a HC comprising CDR-H1, CDR-H2, and CDR-H3 of a HC set forth in any one of SEQ ID NOs: 51, 54, 56, and 58, and having at least 85% amino acid sequence identity thereof, and/or
   a LC comprising CDR-L1, CDR-L2, and CDR-L3 of a LC set forth in SEQ ID NO: 86 or 88, and having at least 85% amino acid sequence identity thereof.
Embodiment 14. The antigen-binding site of embodiment 12 or 13, wherein the sequence identity is at least 90%, 95%, 96%, 97%, 98%, or 99%.
Embodiment 15. The antigen-binding site of any one of embodiments 5 and 8-14, wherein the antigen-binding site comprises
(a) the VH of a HC set forth in SEQ ID NO: 39; and/or the VL of a LC set forth in SEQ ID NO: 44; or
(b) the VH of a HC set forth in SEQ ID NO: 49; and/or the VL of a LC set forth in SEQ ID NO: 50; or
(c) the VH of a HC set forth in any one of SEQ ID NOs: 51, 54, 56, and 58; and/or the VL of a LC set forth in SEQ ID NO: 86 or 88.
Embodiment 16. The antigen-binding site of any one of embodiments 6 and 8-9, wherein the antigen-binding site comprises
(a) the CDR1, CDR2, and/or CDR3 of a HC set forth in any one of SEQ ID NO: 1, 17, 19, 22, 24, 26, or 28; and/or
(b) the CDR1, CDR2, and/or CDR3 of a LC set forth in any one of SEQ ID NO: 9, 18, 30, 35, or 37.
Embodiment 17. The antigen-binding site of any one of embodiments 6, 8-9, and 16, wherein the antigen-binding site comprises
(a) a VH comprising complementarity-determining regions:
   CDR-H1 comprising the amino acid sequence: SYWMH (SEQ ID NO: 3),
   CDR-H2 comprising the amino acid sequence: EIYPTNGGTTYNEKFKR (SEQ ID NO: 4), and
   CDR-H3 comprising the amino acid sequence: EDFYAMDY (SEQ ID NO: 5); and/or
(b) a VL comprising complementarity-determining regions:
   CDR-L1 comprising the amino acid sequence: KSSQSLLDSDGKTCLN (SEQ ID NO: 11);
   CDR-L2 comprising the amino acid sequence: LVSKLDS (SEQ ID NO: 12); and
   CDR-L3 comprising the amino acid sequence: WQGTHFPYT (SEQ ID NO: 13).
Embodiment 18. The antigen-binding site of any one of embodiments 6, 8-9, and 16, wherein the antigen-binding site comprises
(a) a VH comprising complementarity-determining regions:
   CDR-H1 comprising the amino acid sequence: GYAFTSYWMH (SEQ ID NO: 6),
   CDR-H2 comprising the amino acid sequence: EIYPTNGGTT (SEQ ID NO: 7), and
   CDR-H3 comprising the amino acid sequence: EDFYAMDY (SEQ ID NO: 8); and/or
(b) a VL comprising complementarity-determining regions:
   CDR-L1 comprising the amino acid sequence: KSSQSLLDSDGKTCLN (SEQ ID NO: 14);
   CDR-L2 comprising the amino acid sequence: LVSKLDS (SEQ ID NO: 15); and
   CDR-L3 comprising the amino acid sequence: WQGTHFPYT (SEQ ID NO: 16).
Embodiment 19. The antigen-binding site of any one of embodiments 6, 8-9, and 16-18, wherein the antigen-binding site comprises
(a) a VH comprising an amnio acid sequence having at least 85% identity to the VH of a HC set forth in SEQ ID NO: 1; and/or
   a VL comprising an amnio acid sequence having at least 85% identity to the VL of a LC set forth in SEQ ID NO: 9; or
(b) a VH comprising an amnio acid sequence having at least 85% identity to the VH of a HC set forth in SEQ ID NO: 17; and/or
   a VL comprising an amnio acid sequence having at least 85% identity to the VL of a LC set forth in SEQ ID NO: 18; or
(c) a VH comprising an amnio acid sequence having at least 85% identity to the VH of a HC set forth in any one of SEQ ID NOs: 19, 22, 24, 26, and 28; and/or
   a VL comprising an amnio acid sequence having at least 85% identity to the VL of a LC set forth in any one of SEQ ID NOs: 30, 35, and 37.
Embodiment 20. The antigen-binding site of any one of embodiments 6, 8-9, and 16-19, wherein the antigen-binding site comprises
(a) a HC comprising CDR-H1, CDR-H2, and CDR-H3 of a HC set forth in SEQ ID NO: 1, and having at least 85% amino acid sequence identity to SEQ ID NO: 1; and/or
   a LC comprising CDR-L1, CDR-L2, and CDR-L3 of a LC set forth in SEQ ID NO: 9, and having at least 85% amino acid sequence identity to SEQ ID NO: 9; or
(b) a HC comprising CDR-H1, CDR-H2, and CDR-H3 of a HC set forth in SEQ ID NO: 17, and having at least 85% amino acid sequence identity to SEQ ID NO: 17; and/or
   a LC comprising CDR-L1, CDR-L2, and CDR-L3 of a LC set forth in SEQ ID NO: 18, and having at least 85% amino acid sequence identity to SEQ ID NO: 18; or
(c) a HC comprising CDR-H1, CDR-H2, and CDR-H3 of a HC set forth in any one of SEQ ID NOs: 19, 22, 24, 26, and 28, and having at least 85% amino acid sequence identity thereof, and/or
   a LC comprising CDR-L1, CDR-L2, and CDR-L3 of a LC set forth in any one of SEQ ID NOs: 30, 35, and 37, and having at least 85% amino acid sequence identity thereof.
Embodiment 21. The antigen-binding site of embodiment 19 or 20, wherein the sequence identity is at least 90%, 95%, 96%, 97%, 98%, or 99%.
Embodiment 22. The antigen-binding site of any one of embodiments 6, 8-9, and 16-21, wherein the antigen-binding site comprises
(a) the VH of a HC set forth in SEQ ID NO: 1; and/or the VL of a LC set forth in SEQ ID NO: 9; or
(b) the VH of a HC set forth in SEQ ID NO: 17; and/or the VL of a LC set forth in SEQ ID NO: 18; or
(c) the VH of a HC set forth in any one of SEQ ID NOs: 19, 22, 24, 26, and 28; and/or the VL of a LC set forth in any one of SEQ ID NOs: 30, 35, and 37.
Embodiment 23. The antigen-binding site of any one of embodiments 6-9, wherein the antigen-binding site comprises
(a) the CDR1, CDR2, and/or CDR3 of a VH set forth in SEQ ID NO: 121; and/or
(b) the CDR1, CDR2, and/or CDR3 of a LC set forth in SEQ ID NO: 126.
Embodiment 24. The antigen-binding site of any one of embodiments 6-9 and 23, wherein the antigen-binding site comprises
(a) a VH comprising complementarity-determining regions:
   CDR-H1 comprising the amino acid sequence: DYGMH (SEQ ID NO: 122),
   CDR-H2 comprising the amino acid sequence: GISWNSGSIGYADSVKG (SEQ ID NO: 123), and
   CDR-H3 comprising the amino acid sequence: GGQTIDI (SEQ ID NO: 124); and/or
(b) a VL comprising complementarity-determining regions:
   CDR-L1 comprising the amino acid sequence: RASQSISSYLN (SEQ ID NO: 127);
   CDR-L2 comprising the amino acid sequence: AASSLQS (SEQ ID NO: 128); and
   CDR-L3 comprising the amino acid sequence: QQSYSTPFT (SEQ ID NO: 129).
Embodiment 25. The antigen-binding site of any one of embodiments 6-9 and 23-24, wherein the antigen-binding site comprises
   a VH comprising an amnio acid sequence having at least 85% to SEQ ID NO: 121; and/or
   a VL comprising an amnio acid sequence having at least 85% to SEQ ID NO: 126.
Embodiment 26. The antigen-binding site of any one of embodiments 6-9 and 23-25, wherein the antigen-binding site comprises
   a HC comprising CDR-H1, CDR-H2, and CDR-H3 of a HC set forth in SEQ ID NO: 120, and having at least 85% amino acid sequence identity to SEQ ID NO: 120; and/or
   a LC comprising CDR-L1, CDR-L2, and CDR-L3 of a LC set forth in SEQ ID NO: 125, and having at least 85% amino acid sequence identity to SEQ ID NO: 125.
Embodiment 27. The antigen-binding site of embodiment 25 or 26, wherein the sequence identity is at least 90%, 95%, 96%, 97%, 98%, or 99%.
Embodiment 28. The antigen-binding site of any one of embodiments 6-9 and 23-27, wherein the antigen-binding site comprises a VH comprising an amnio acid sequence set forth in SEQ ID NO: 121; and/or a VL comprising an amnio acid sequence set forth in SEQ ID NO: 126.
Embodiment 29. An antibody or antigen-binding fragment thereof comprising an antigen-binding site of any one of embodiments 1-4.
Embodiment 30. An antibody or antigen-binding fragment thereof comprising an antigen-binding site of any one of embodiments 5 and 10-15.
Embodiment 31. The antibody or antigen-binding fragment thereof of embodiment 30, capable of blocking c-kit signaling.
Embodiment 32. An antibody or antigen-binding fragment thereof comprising an antigen-binding site of any one of embodiments 6 and 16-22.
Embodiment 33. The antibody or antigen-binding fragment thereof of embodiment 32, capable of agonizing siglec-6.
Embodiment 34. An antibody or antigen-binding fragment thereof comprising an antigen-binding site of embodiment 7.
Embodiment 35. An antibody or antigen-binding fragment thereof comprising an antigen-binding site of any one of embodiments 23-28.
Embodiment 36. The antibody or antigen-binding fragment thereof of any one of embodiments 29-35, wherein the antibody is a humanized antibody.
Embodiment 37. The antibody or antigen-binding fragment thereof of any one of embodiments 29-35, wherein the antibody is a human antibody.
Embodiment 38. The antibody or antigen-binding fragment thereof of any one of embodiments 29-35, wherein the antibody is a multispecific antibody.
Embodiment 39. The antibody or antigen-binding fragment thereof of any one of embodiments 29-35, wherein the antibody is a bispecific antibody.
Embodiment 40. The antibody or antigen binding fragment thereof of any one of embodiments 29-35, wherein the antigen binding fragment is a Fab, a F(ab′)2, a Fab′, an scFv, an Fv fragment, a Fd fragment, or a diabody.
Embodiment 41. The antibody or antigen binding fragment thereof of any one of embodiments 29-35, wherein the antibody or antigen binding fragment thereof comprises an antibody heavy chain constant region.
Embodiment 42. The antibody or antigen binding fragment thereof of embodiment 41, wherein the antibody heavy chain constant region is a human IgG heavy chain constant region.
Embodiment 43. The antibody or antigen binding fragment thereof of any one of embodiments 29-42, wherein the antibody or antigen binding fragment thereof has reduced or null effector function.
Embodiment 44. The antibody or antigen binding fragment thereof of any one of embodiments 29-43, wherein the antibody or antigen binding fragment thereof is afucosylated.
Embodiment 45. The antibody or antigen binding fragment thereof of embodiment 44, wherein the antibody or antigen binding fragment thereof is at least about 75% afucosylated.
Embodiment 46. The antibody or antigen-binding fragment thereof of embodiment 44 or 45, wherein the antibody or antigen binding fragment thereof is capable of depleting mast cell.
Embodiment 47. A protein comprising an antigen-binding site of any one of embodiments 1-4.
Embodiment 48. A protein comprising an antigen-binding site of any one of embodiments 5 and 10-15.
Embodiment 49. A protein comprising an antigen-binding site of any one of embodiments 6 and 16-22.
Embodiment 50. A protein comprising an antigen-binding site of embodiment 7.
Embodiment 51. A protein comprising an antigen-binding site of any one of embodiments 23-28
Embodiment 52. A protein comprising two or more antigen-binding sites selected from: (i) an antigen-binding site of any one of embodiments 1-4; (ii) an antigen-binding site of any one of embodiments 5 and 10-15; (iii) an antigen-binding site of any one of embodiments 6 and 16-22; (iv) an antigen-binding site of embodiment 7; and (v) an antigen-binding site of any one of embodiments 23-28.
Embodiment 53. A protein comprising two antigen-binding sites selected from: (i) an antigen-binding site of any one of embodiments 1-4; (ii) an antigen-binding site of any one of embodiments 5 and 10-15; (iii) an antigen-binding site of any one of embodiments 6 and 16-22; (iv) an antigen-binding site of embodiment 7; and (v) an antigen-binding site of any one of embodiments 23-28.
Embodiment 54. A protein comprising
(i) an antigen-binding site of any one of embodiments 5 and 10-15; and
(ii) an antigen-binding site selected from: (a) an antigen-binding site of embodiment 2, (b) an antigen-binding site of embodiment 3, or (c) an antigen-binding site of one of embodiments 6 and 16-22.
Embodiment 55. The protein of any one of embodiments 47-54, further comprising an antibody heavy chain constant region.
Embodiment 56. The protein of embodiment 55, wherein the antibody heavy chain constant region is a human IgG heavy chain constant region.
Embodiment 57. The protein of any one of embodiments 47-56, wherein the protein has reduced or null effector function.
Embodiment 58. The protein of any one of embodiments 47-57, wherein the protein is afucosylated.
Embodiment 59. The protein of embodiment 58, wherein the protein is at least about 75% afucosylated.
Embodiment 60. The protein of embodiment 58 or 59, wherein the protein is capable of depleting mast cell.
Embodiment 61. An isolated nucleic acid encoding an antigen-binding site of any one of embodiments 1-28, an antibody or antigen-binding fragment thereof of any one of embodiments 29-46, or a protein of any one of embodiments 47-60.
Embodiment 62. An expression vector comprising the isolated nucleic acid of embodiment 61.
Embodiment 63. A host cell comprising the isolated nucleic acid molecule of embodiment 61 or the expression vector of embodiment 62.
Embodiment 64. A pharmaceutical composition comprising an antigen-binding site of any one of embodiments 1-28, an antibody or antigen-binding fragment thereof of any one of embodiments 29-46, or a protein of any one of embodiments 47-60, and a pharmaceutically acceptable carrier.
Embodiment 65. A method of treating a disease or disorder in a subject in need thereof, comprising administering to the subject an effective amount of an antigen-binding site of any one of embodiments 1-28, an antibody or antigen-binding fragment thereof of any one of embodiments 29-46, a protein of any one of embodiments 47-60, or a pharmaceutical composition of embodiment 64.
Embodiment 66. The method of embodiment 65, wherein the disease or disorder is a mast cell associated disease or disorder.
Embodiment 67. The method of embodiment 65, wherein the disease or disorder is an autoimmune disease.
Embodiment 68. The method of embodiment 65, wherein the disease or disorder is an allergy.
Embodiment 69. The method of embodiment 65, wherein the disease or disorder is an inflammation.
Embodiment 70. The method of embodiment 65, wherein the disease or disorder is pruritus.
Embodiment 71. The method of embodiment 65, wherein the disease or disorder is a cancer.
Embodiment 72. The antibody or antigen-binding fragment thereof of embodiment 12 or 13 that comprises an amino acid sequence selected from any one of SEQ ID NOs: 1, 9, 17-19, 22, 24, 26, 28, 30, 35, 37, 120, 121, 125, and 126, or a fragment thereof.
Embodiment 73. The antibody or antigen-binding fragment thereof of embodiment 12 or 13 that comprises at least one amino acid sequence selected from any one of SEQ ID NOs: 1, 9, 17-19, 22, 24, 26, 28, 30, 35, 37, 120, 121, 125, and 126, or a fragment thereof.
Embodiment 74. The antibody or antigen-binding fragment thereof of embodiment 12 or 13 that comprises at least one CDR sequence found in sequences selected from any one of SEQ ID NOs: 1, 9, 17-19, 22, 24, 26, 28, 30, 35, 37, 120, 121, 125, and 126.
Embodiment 75. The antibody or antigen-binding fragment thereof of embodiment 12 or 13 that comprises at least two CDR sequences found in sequences selected from any one of SEQ ID NOs: 1, 9, 17-19, 22, 24, 26, 28, 30, 35, 37, 120, 121, 125, and 126.
Embodiment 76. The antibody or antigen-binding fragment thereof of embodiment 12 or 13 that comprises at least three CDR sequences found in sequences selected from any one of SEQ ID NOs: 1, 9, 17-19, 22, 24, 26, 28, 30, 35, 37, 120, 121, 125, and 126.
Embodiment 77. The antibody or antigen-binding fragment thereof of embodiment 11 that comprises an amino acid sequence selected from any one of SEQ ID NOs: 39, 44, 49-51, 54, 56, 58, 86, and 88, or a fragment thereof.
Embodiment 78. The antibody or antigen-binding fragment thereof of embodiment 11 that comprises at least one amino acid sequence selected from any one of SEQ ID NOs: 39, 44, 49-51, 54, 56, 58, 86, and 88, or a fragment thereof.
Embodiment 79. The antibody or antigen-binding fragment thereof of embodiment 11 that comprises at least two amino acid sequences selected from any one of SEQ ID NOs: 39, 44, 49-51, 54, 56, 58, 86, and 88, or a fragment thereof.
Embodiment 80. The antibody or antigen-binding fragment thereof of embodiment 11 that comprises at least one CDR found in a sequence selected from any one of SEQ ID NOs: 39, 44, 49-51, 54, 56, 58, 86, and 88, or a fragment thereof.
Embodiment 81. The antibody or antigen-binding fragment thereof of embodiment 11 that comprises at least two CDRs found in sequences selected from any one of SEQ ID NOs: 39, 44, 49-51, 54, 56, 58, 86, and 88, or a fragment thereof.
Embodiment 82. The antibody or antigen-binding fragment thereof of embodiment 11 that comprises at least three CDRs found in sequences selected from any one of SEQ ID NOs: 39, 44, 49-51, 54, 56, 58, 86, and 88, or a fragment thereof.
Embodiment 83. An antibody or antigen-binding fragment that comprises an amino acid sequence selected any one of SEQ ID NOs: 1, 9, 17-19, 22, 24, 26, 28, 30, 35, 37, 39, 44, 49-51, 54, 56, 58, 86, 88, 120, 121, 125, and 126, or a fragment thereof.

### EXAMPLES

### Example 1. Antibody Production

FCER1, c-Kit, and siglec (e.g., siglec-6 and siglec 8) targeting human antibodies are produced by methods known in the art.

In some embodiments, transgenic mice that are incapable of expressing functional endogenous immunoglobulins but express human immunoglobulin genes are used. In particular, the human heavy and light chain immunoglobulin gene complexes are introduced randomly or by homologous recombination into mouse embryonic stem cells. Alternatively, the human variable region, constant region, and diversity region are introduced into mouse embryonic stem cells in addition to the human heavy and light chain genes. The mouse heavy and light chain immunoglobulin genes are rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of an antigen (e.g., FCER1, c-Kit, or sigle). Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation.

In some other embodiments, FCER1, c-Kit, and siglec (e.g., siglec-6 and siglec 8) targeting antibodies are produced by screening yeast display libraries.

Further, c-Kit/siglec-6 bispecific antibodies are produced by methods known in the art. In some embodiments, c-Kit/siglec-6 bispecific antibodies are in CrossMab format.

### Example 2. Binding of Antibodies Toward c-Kit

Target binding of c-Kit antibodies to c-Kit-expressing cells was assessed by flow cytometry. Cultured M-07e cells were resuspended in FACS Blocking Buffer (PBS+10% normal human serum) for 30 minutes. Following washing, cells were incubated with c-Kit antibodies in FACS Buffer (PBS+0.5% bovine serum albumin+0.09% sodium azide) and incubated at 4° C. for 30 minutes, washed and incubated with PE-labeled anti-human secondary antibody. Cells were incubated at 4° C. for 30 minutes in the dark. Following washing, cytofluorimetric analysis was conducted using a BD FACS Symphony A3 (BD Biosciences). Cells were sorted by scatter properties and separated for single cell analysis using side scatter width vs side scatter area. Antibody binding to cells was quantitated by measuring fluorescent emissions of excitation wavelength of 561 nm using a 586/15 nm filter. Data analysis was performed using FlowJo Software and summarized in Table 2. All tested c-Kit antibodies were capable of binding to cells expressing c-Kit.

TABLE 2
Binding of c-Kit antibodies to cell surface
expressed c-Kit on M-07e cells

| | SEQ | Percent Positive for | Measuring Fluorescent |
|---|---|---|---|
| Antibody | ID NO | c-Kit on M-07e cells | Intensity (MFI) |
| hSR1-1 | 51 and 86 | 99.5 | 5021 |
| hSR1-2 | 54 and 86 | 99.7 | 5199 |
| hSR1-3 | 56 and 86 | 99.8 | 5187 |
| hSR1-4 | 58 and 86 | 99.8 | 4962 |
| hSR1-5 | 51 and 88 | 99.5 | 4029 |
| hSR1-6 | 54 and 88 | 99.4 | 3591 |
| chSR1 | 49 and 50 | 99.7 | 5344 |
| hIgG1 control | | 2.3 | 85 |

Further, binding affinities of c-Kit antibodies to human c-Kit and cynomolgus c-Kit were measured by biolayer interferometry (BLA) using an Octect instrument. Briefly, capture sensors were loaded with test antibodies with 3-fold dilutions. The association and dissociation rates to were determined and used to calculate the equilibrium dissociation constant K_{D} (Table 3). Affinities to c-Kit measured by K_{D} values were observed in the subnanomolar range for tested humanized c-Kit antibodies (hSR-1-1 to hSR1-6) and in the single digit nanomolar range for tested chimeric c-Kit antibody (chSR1).

TABLE 3
Binding affinities for human and cynonolgus
c-Kit of determined by BLI

Affinity (KD)

| Antibody | Human c-Kit | Cynomoguls c-Kit |
|---|---|---|
| hSR1-1 | 1.99E−10 | 2.320E−10 |
| hSR1-2 | 2.45E−10 | 3.885E−10 |
| hSR1-3 | 2.56E−10 | 3.323E−10 |
| hSR1-4 | 3.86E−10 | 5.633E−10 |
| hSR1-5 | 2.56E−10 | 4.045E−10 |
| hSR1-6 | 2.47E−10 | 2.959E−10 |
| chSR1 | 1.89E−09 | 3.338E−09 |

### Example 3. Cross-Reactivity Non-Specific Binding of c-Kit Antibodies Analyzed by ELISA

An ELISA-based assay was employed to assess the cross-reactivity and non-specific binding of c-Kit antibodies toward human c-Kit, cynomongus c-Kit, and c-Kit family members.

His-tagged human c-Kit, His-tagged cynomolgus c-Kit, and His-tagged PDGFRa, PDGFRb, CSF1R, and FLT3 extracellular domains were immobilized on 96 well ELISA plates. Plates were washed with PBST (Phosphate buffered saline+0.05% Tween 20) and blocked for 1 hour with 1% BSA in PBS. Following washing with PBST, mAbs were added to the coated plate and incubated for 2 hours at room temperature. Plates were washed and incubated with HRP-conjugated goat anti-human IgG secondary antibody for 1 hour at room temperature. Following washing, antibody binding was detected colorimetrically by adding 3,3′,5,5′-tetramethylbenzidine (TMB) and incubated at room temperature for 10-20 minutes before adding 2.5N sulfuric acid stopping solution. Absorbance at 450 nm was measured on a plate reader (PerkinElmer). Data was analyzed using GraphPad Prism.

Results for exemplary c-Kit antibodies are depicted in Table 4. The c-Kit antibodies were capable of binding to both human and cynomolgus c-Kit but did not cross react with c-Kit family members PDGFRa, PDGFRb, CSF1R, or FLT3.

TABLE 4
Cross-reactivity of humanized and chimeric c-Kit antibodies

| | Cross Reactivity | | |
|---|---|---|---|
| Antibody | Human c-Kit | Cyno c-Kit | c-Kit Family members |
| hSR1-1 | + | + | − |
| hSR1-2 | + | + | − |
| hSR1-3 | + | + | − |
| hSR1-4 | + | + | − |
| hSR1-5 | + | + | − |
| hSR1-6 | + | + | − |
| chSR1 | + | + | − |

### Example 4. Binding of Antibodies Toward Siglec-6

Target binding of siglec-6 antibodies to siglec-6-expressing cells was assessed by flow cytometry. Cultured HMC1.2 cells were resuspended in FACS Blocking Buffer (PBS+10% normal human serum) for 30 minutes. Following washing, cells were incubated with siglec-6 antibodies in FACS Buffer (PBS+0.5% bovine serum albumin+0.09% sodium azide) and incubated at 4° C. for 30 minutes, washed and incubated with PE-labeled anti-human or anti-mouse secondary antibodies. Cells were incubated at 4° C. for 30 minutes in the dark. Following washing, cytofluorimetric analysis was conducted using a BD FACS Symphony A3 (BD Biosciences). Cells were sorted by scatter properties and separated for single cell analysis using side scatter width vs side scatter area. Antibody binding to cells was quantitated by measuring fluorescent emissions of excitation wavelength of 561 nm using a 586/15 nm filter. Data analysis was performed using FlowJo Software and summarized in Table 5. All tested siglec-6 antibodies were capable of binding to cells expressing siglec-6.

TABLE 5
Binding of humanized and chimeric siglec-6 antibodies
to cell surface expressed siglec-6 on HMC1.2 cells

| | SEQ | Percent Positive for Siglec-6 | |
|---|---|---|---|
| Antibody | ID NO | on HMC1.2 cells | MFI |
| hRND-2 | 22 and 37 | 99.9 | 3134 |
| hRND-3 | 24 and 37 | 99.9 | 3695 |
| hRND-4 | 26 and 37 | 100.0 | 3203 |
| hRND-5 | 28 and 37 | 99.9 | 1942 |
| chRND | 17 and 37 | 100.0 | 3608 |
| mRND | 1 and 9 | 99.9 | 1974 |
| hIgG1 control | | 0.1 | 212 |

Further, binding affinities of siglec-6 antibodies to human siglec-6 were measured by BLA using an Octect instrument. Briefly, capture sensors were loaded with test antibodies with 3-fold dilutions. The association and dissociation rates to were determined and used to calculate the equilibrium dissociation constant K_{D} (Table 6). Affinities to siglec-6 measured by K_{D} values were observed in the subnanomolar range for tested humanized and chimeric siglec-6 antibodies.

TABLE 6
Binding affinities for human siglect-6 of determined by BLI

| | Affinity (KD) |
|---|---|
| Antibody | Human siglec-6 |
| hRND-2 | 1.70E−10 |
| hRND-3 | 1.37E−10 |
| hRND-4 | 3.15E−10 |
| hRND-5 | 1.66E−10 |
| chRND | 1.01E−10 |

### Example 5. Cross-Reactivity Non-Specific Binding of Siglec-6 Antibodies Analyzed by ELISA

An ELISA-based assay was employed to assess the cross-reactivity and non-specific binding of siglec-6 antibodies toward human siglec-6, cynomongus siglec-6, and siglec-6 family members siglec 1-5, 7-12, 14, and 15.

Cultured HEK293 cells expressing human siglec-6, cynomolgus siglec-6, or siglec 6 family members were resuspended in FACS Blocking Buffer (PBS+10% normal human serum) for 30 minutes. Following washing, cells were incubated with siglec-6 antibodies in FACS Buffer (PBS+0.5% bovine serum albumin+0.09% sodium azide) and incubated at 4° C. for 30 minutes, washed and incubated with PE-labeled anti-human secondary antibody. Cells were incubated at 4° C. for 30 minutes in the dark. Following washing, cytofluorimetric analysis was conducted using a BD FACS Symphony A3 (BD Biosciences). Cells were sorted by scatter properties and separated for single cell analysis using side scatter width vs side scatter area. Antibody binding to cells was quantitated by measuring fluorescent emissions of excitation wavelength of 561 nm using a 586/15 nm filter. Data analysis was performed using FlowJo Software.

Results for exemplary siglec-6 antibodies are depicted in Table 7. The siglec-6 antibodies were capable of binding to both human and cynomolgus siglec-6 but did not cross react with siglec-6 family members siglec 1-5, 7-12, 14, or 15.

TABLE 7
Cross-reactivity of humanized and chimeric siglec-6 antibodies

| | Cross Reactivity | | |
|---|---|---|---|
| Antibody | Human siglec-6 | Cyno siglec-6 | Siglec-6 Family members |
| hRND-2 | + | + | − |
| hRND-3 | + | + | − |
| hRND-4 | + | + | − |
| hRND-5 | + | + | − |
| hRND-6 | + | + | − |
| chRND | + | + | − |

### Example 6. Inhibition of Cell Proliferation by c-Kit Antibodies

The ability of c-Kit antibodies to inhibit stem cell factor (SCF)-dependent cell proliferation was assessed.

M-07e cells were grown according to distributor's recommendation. Cells were starved of SCF overnight (16 hours). SCF starved cells were plated at a density of 15,000 cells per well of a 96-well plate in media without SCF. C-Kit antibodies (SR1 antibodies or CDX-0159) or controls were titrated onto cells and the plate was incubated for 2 hours at 37° C., 5% CO₂. Following the 2-hour incubation, SCF was added to a final concentration of 50 ng/mL and plates were incubated for 72 hours at 37° C., 5% CO₂. Cell viability was measured using Cell Titer Glo (Promega). Cell Titer Glo was added to each well and the plate was incubated for 2 minutes protected from light on a benchtop shaker. Luminescence was measured on a plate reader (PerkinElmer). Data were analyzed using GraphPad Prism.

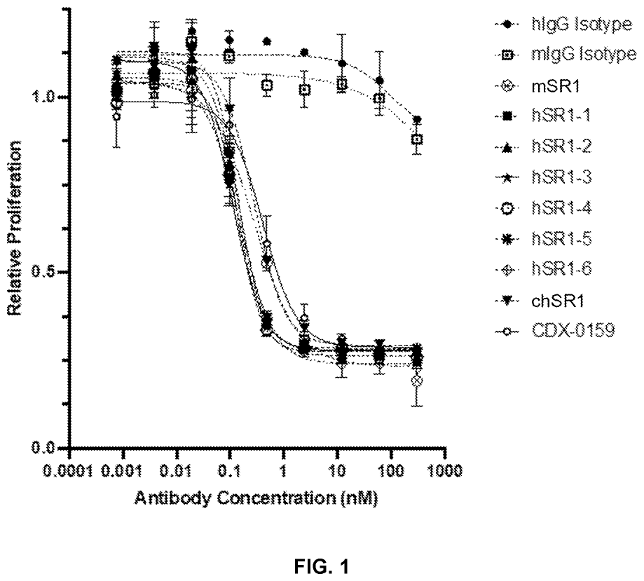

Exemplary results are depicted in FIG. 1, in which antibody concentrations were plotted as a function of relative luminescence and curve fitting was performed using a four-parameter non-linear regression algorithm. IC₅₀ values were determined and summarized in

TABLE 8
Inhibition of SCF-dependent proliferation of M-07e cells

| Antibody | IC₅₀ (nM) |
|---|---|
| muSR1 | 0.2828 |
| hSR1-1 | 0.1415 |
| hSR1-2 | 0.1417 |
| hSR1-3 | 0.1129 |
| hSR1-4 | 0.1385 |
| hSR1-5 | 0.1415 |
| hSR1-6 | 0.1517 |
| chSR1 | 0.2769 |
| CDX-0159 | 0.4082 |

### Example 7. Inhibition of Cell Proliferation by c-Kit/Siglec 6 Bispecific Antibodies

The ability of c-Kit/siglec-6 antibodies to inhibit SCF-dependent cell proliferation was assessed.

M-07e cells were starved of SCF overnight (16 hours). SCF starved cells were plated at a density of 15,000 cells per well of a 96-well plate in media without SCF. Anti-siglec-6 JML-1 and hRND-3, anti-c-Kit chSR1 and CDX-0159, and anti-siglec/anti-c-Kit bispecific JML-1/hSR1-1 and hRND-3/hSR1-1 were titrated onto cells and incubated for 2 hours at 37° C., 5% CO₂. Following the 2-hour incubation, SCF was added to a final concentration of 50 ng/mL and plates were incubated for 72 hours at 37° C., 5% CO₂. Cell viability was measured using Cell Titer Glo (Promega). Cell Titer Glo was added to each well and the plate was incubated for 2 minutes protected from light on a benchtop shaker. Luminescence was measured on a plate reader (PerkinElmer). Data were analyzed using GraphPad Prism with curve fitting performed using a four-parameter non-linear regression algorithm. Relative proliferation was calculated by comparing the mean relative fluorescence units (RFU) from each sample (cells+antibody+SCF) to mean RFU of cells incubated with SCF alone.

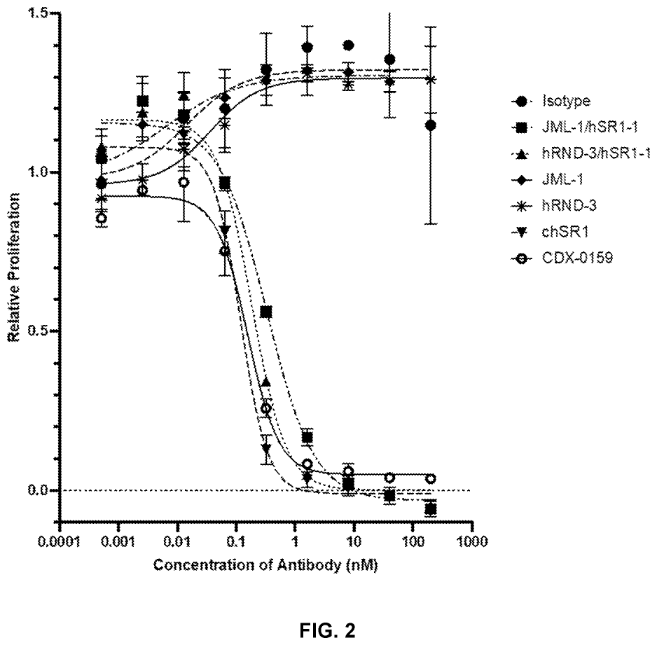

Results are shown in FIG. 2. Tested c-Kit/siglec-6 bispecific antibodies inhibited SCF-dependent proliferation in M-07e cells; siglect-6 monospecific antibodies did not inhibit proliferation.

### Example 8. Mast Cell Degranulation Assay

Experiment was performed to evaluate whether disclosed siglect-6 antibodies induce mast cell degranulation.

CD34+ progenitor cells from whole blood were cultured in Mast Cell Media containing cytokines and growth factors. Cultures matured into mast cells in 6-8 weeks from the start of the differentiation process.

To induce FcγRI expression in mast cells, cells were treated with IFN-7 at 100 UI/mL for 72 hours. Mast cells with and without IFN-7 treatment were starved of cytokines for 4 hours. Cells were washed in Tyrode's buffer and plated at 35,000 cells per well in a 96-well plate. The plate was incubated for 30 minutes at 37° C. and 5% CO₂. Siglec 6 antibodies with normal human IgG1 Fc, effector silent IgG1 Fc (containing L234A/L235A/P329G mutations), or effector enhanced IgG1 Fc (afucosylated) and PMA/Ionomycin were added to appropriate wells along with SCF and the plate incubated for 30 minutes at 37° C. (in air). Media from the treated mast cell cultures was added to a 96 well plate containing PNAG solution (0.35% p-nitrophenyl N-acetyl-b-D-glucosamide) and incubated for 90 minutes at 37° C. (in air). Similarly, the remaining mast cells were lysed in 0.1% triton X-100 and added to PNAG solution for 90 minutes. Glycine buffer (400 mM, pH10.7) was added to all wells and absorbance at 405 nm was read in a plate reader. β-Hexosaminidase was measured by calculating the percent β-hexosaminidase released from the β-hexosaminidase contained by mast cells.

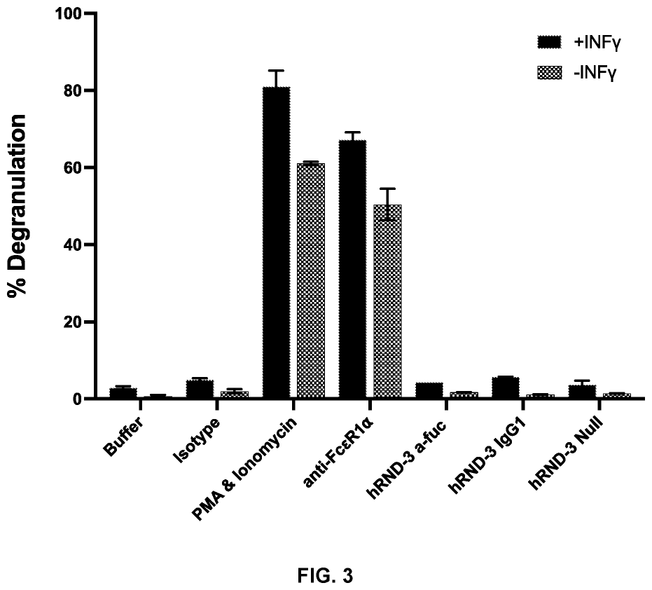

Results are shown in FIG. 3. All three Siglec-6 hRND-3 antibodies with modifications in their Fc domains did not induce degranulation of CD34 derived Mast cells

### SEQUENCE ANNEX

In some sequences, variable regions are bolded, and CDRs are underlined.

TABLE 9
Sequences of exemplary antigen-binding sites capable of binding siglect-6

| | | |
|---|---|---|
| SEQ | | |
| ID NO | Description | Sequence |

Murine anti-siglec-6 RND (mRND)

| | | |
|---|---|---|
| 1 | Heavy chain | QVQLQQPGAELVKPGASVKLSCKASGYAFTSYWMHWVKLRPGQGFEWI |
| | | GEIYPTNGGTTYNEKFKRKATLTVDKSSSTAYMQLSSLTSEDSAVYYC |
| | | TIEDFYAMDYWGQGTSVTVSSAKTTAPSVYPLAPVCGDTTGSSVTLGC |
| | | LVKGYFPEPVTLTWNSGSLSSGVHTFPAVLQSDLYTLSSSVTVTSSTW |
| | | PSQSITCNVAHPASSTKVDKKIEPRGPTIKPCPPCKCPAPNLLGGPSV |
| | | FIFPPKIKDVLMISLSPIVTCVVVDVSEDDPDVQISWFVNNVEVHTAQ |
| | | TQTHREDYNSTLRVVSALPIQHQDWMSGKEFKCKVNNKDLPAPIERTI |
| | | SKPKGSVRAPQVYVLPPPEEEMTKKQVTLTCMVTDEMPEDIYVEWINN |
| | | GKTELNYKNTEPVLDSDGSYFMYSKLRVEKKNWVERNSYSCSVVHEGL |
| | | HNHHTTKSFSRTPGK |
| 2 | Heavy chain variable | QVQLQQPGAELVKPGASVKLSCKASGYAFTSYWMHWVKLRPGQGFEWI |
| | region | GEIYPTNGGTTYNEKFKRKATLTVDKSSSTAYMQLSSLTSEDSAVYYC |
| | | TIEDFYAMDYWGQGTSVTVSS |
| 3 | HCDR1 (Kabat) | SYWMH |
| 4 | HCDR2 (Kabat) | EIYPTNGGTTYNEKFKR |
| 5 | HCDR3 (Kabat) | EDFYAMDY |
| 6 | HCDR1 (AbM) | GYAFTSYWMH |
| 7 | HCDR2 (AbM) | EIYPTNGGTT |
| 8 | HCDR3 (AbM) | EDFYAMDY |
| 9 | Light chain | DVVMTQTPLTLSVTIGQPASISCKSSQSLLDSDGKTCLNWLLQRPGQS |
| | | PKRLIYLVSKLDSGVPDRFTGSGSGTDFTLKISRVEAEDLGVYYCWQG |
| | | THEPYTFGGGTKLEIKRADAAPTVSIFPPSSEQLTSGGASVVCFINNE |
| | | YPKDINVKWKIDGSERQNGVLNSWTDQDSKDSTYSMSSTLTLTKDEYE |
| | | RHNSYTCEATHKTSTSPIVKSENRNEC |
| 10 | Light chain variable | DVVMTQTPLTLSVTIGQPASISCKSSQSLLDSDGKTCLNWLLQRPGQS |
| | region | PKRLIYLVSKLDSGVPDRFTGSGSGTDFTLKISRVEAEDLGVYYCWQG |
| | | THEPYTFGGGTKLEIKR |
| 11 | LCDR1 (Kabat) | KSSQSLLDSDGKTCLN |
| 12 | LCDR2 (Kabat) | LVSKLDS |
| 13 | LCDR3 (Kabat) | WQGTHEPYT |
| 14 | LCDR1 (AbM) | KSSQSLLDSDGKTCLN |
| 15 | LCDR2 (AbM) | LVSKLDS |
| 16 | LCDR3 (AbM) | WQGTHEPYT |

Chimeric anti-siglec-6 RND (chRND)

| | | |
|---|---|---|
| 17 | Heavy chain | QVQLQQPGAELVKPGASVKLSCKASGYAFTSYWMHWVKLRPGQGFEWI |
| | (Fc or constant region | GETYPTNGGTTYNEKFKRKATLTVDKSSSTAYMQLSSLTSEDSAVYYC |
| | may be of any IgG | TIEDFYAMDYWGQGTSVTVSSASTKGPSVFPLAPSSKSTSGGTAALGC |
| | isotype) | LVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPELLGGPSV |
| | | FLFPPKPKDTLMISRTPEVTCVVVDVSHEDPEVKENWYVDGVEVHNAK |
| | | TKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEAL |
| | | HNHYTQKSLSLSPGK |

Humanized anti-siglec-6 RND heavy chain 1 (hRND-H1)

| | | |
|---|---|---|
| 18 | Light chain | DVVMTQTPLTLSVTIGQPASISCKSSQSLLDSDGKTCLNWLLQRPGQS |
| | | PKRLIYLVSKLDSGVPDRFTGSGSGTDFTLKISRVEAEDLGVYYCWQG |
| | | THFPYTFGGGTKLEIKRTVAAPSVFIFPPSDEQLKSGTASVVCLLNNE |
| | | YPREAKVQWKVDNALQSGNSQESVTEQDSKDSTYSLSSTLTLSKADYE |
| | | KHKVYACEVTHQGLSSPVTKSENRGEC |
| 19 | hRND-H1, IgG1 | QVQLVQSGAEVKKPGASVKVSCKASGYAFTSYWMHWVRQAPGQRLEWM |
| | (Fc or constant region | GETYPTNGGTTYAQKFQGRVTMTRDTSTSTVYMELSRLRSDDTAVYYC |
| | may be of any IgG | AREDFYAMDYWGQGTTVTVSSASTKGPSVFPLAPSSKSTSGGTAALGC |
| | isotype) | LVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPELLGGPSV |
| | | FLFPPKPKDTLMISRTPEVTCVVVDVSHEDPEVKENWYVDGVEVHNAK |
| | | TKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEAL |
| | | HNHYTQKSLSLSPG |
| 20 | Heavy chain variable | QVQLVQSGAEVKKPGASVKVSCKASGYAFTSYWMHWVRQAPGQRLEWM |
| | region | GEIYPTNGGTTYAQKFQGRVTMTRDTSTSTVYMELSRLRSDDTAVYYC |
| | | AREDFYAMDYWGQGTTVTVSS |
| 3 | HCDR1 (Kabat) | SYWMH |
| 21 | HCDR2 (Kabat) | EIYPTNGGTTYAQKFQG |
| 5 | HCDR3 (Kabat) | EDFYAMDY |
| 6 | HCDR1 (AbM) | GYAFTSYWMH |
| 7 | HCDR2 (AbM) | EIYPTNGGTT |
| 8 | HCDR3 (AbM) | EDFYAMDY |

Humanized anti-siglec-6 RND heavy chain 2 (hRND-H2)

| | | |
|---|---|---|
| 22 | hRND-H2, IgG1 | QVQLVQSGAEVKKPGASVKVSCKASGYAFTSYWMHWVRQAPGQRLEWM |
| | (Fc or constant region | GETYPTNGGTTYAQKFQGRVTMTVDKSTSTAYMELSRLRSDDTAVYYC |
| | may be of any IgG | TIEDFYAMDYWGQGTTVTVSSASTKGPSVFPLAPSSKSTSGGTAALGC |
| | isotype) | LVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPELLGGPSV |
| | | FLFPPKPKDTLMISRTPEVTCVVVDVSHEDPEVKENWYVDGVEVHNAK |
| | | TKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESN |
| | | GQPENNYKTTPPVLDSDGSFFLYSKLTVDKSRWQQGNVESCSVMHEAL |
| | | HNHYTQKSLSLSPG |
| 23 | Heavy chain variable | QVQLVQSGAEVKKPGASVKVSCKASGYAFTSYWMHWVRQAPGQRLEWM |
| | region | GETYPTNGGTTYAQKFQGRVTMTVDKSTSTAYMELSRLRSDDTAVYYC |
| | | TIEDFYAMDYWGQGTTVTVSS |
| 3 | HCDR1 (Kabat) | SYWMH |
| 21 | HCDR2 (Kabat) | EIYPTNGGTTYAQKFQG |
| 5 | HCDR3 (Kabat) | EDFYAMDY |
| 6 | HCDR1 (AbM) | GYAFTSYWMH |
| 7 | HCDR2 (AbM) | EIYPTNGGTT |
| 8 | HCDR3 (AbM) | EDFYAMDY |

Humanized anti-siglec-6 RND heavy chain 3 (hRND-H3)

| | | |
|---|---|---|
| 24 | huRND-H3, IgG1 | QVQLVQSGAEVKKPGASVKLSCKASGYAFTSYWMHWVRQAPGQRLEWI |
| | (Fc or constant region | GEIYPTNGGTTYAQKFQGRATLTVDKSTSTAYMELSRLRSDDTAVYYC |
| | may be of any IgG | TIEDFYAMDYWGQGTTVTVSSASTKGPSVFPLAPSSKSTSGGTAALGC |
| | isotype) | LVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPELLGGPSV |
| | | FLFPPKPKDTLMISRTPEVTCVVVDVSHEDPEVKENWYVDGVEVHNAK |
| | | TKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEAL |
| | | HNHYTQKSLSLSPG |
| 25 | Heavy chain variable | QVQLVQSGAEVKKPGASVKLSCKASGYAFTSYWMHWVRQAPGQRLEWI |
| | region | GETYPTNGGTTYAQKFQGRATLTVDKSTSTAYMELSRLRSDDTAVYYC |
| | | TIEDFYAMDYWGQGTTVTVSS |
| 3 | HCDR1 (Kabat) | SYWMH |
| 21 | HCDR2 (Kabat) | EIYPTNGGTTYAQKFQG |
| 5 | HCDR3 (Kabat) | EDFYAMDY |
| 6 | HCDR1 (AbM) | GYAFTSYWMH |
| 7 | HCDR2 (AbM) | EIYPTNGGTT |
| 8 | HCDR3 (AbM) | EDFYAMDY |

Humanized anti-siglec-6 RND heavy chain 2b (hRND-H2b)

| | | |
|---|---|---|
| 26 | hRND-H2b, IgG1 | QVQLVQSGAEVKKPGASVKVSCKASGYAFTSYWMHWVRQAPGQRLEWM |
| | (Fc or constant region | GEIYPTNGGTTYNEKFKRRVTMTVDKSTSTAYMELSRLRSDDTAVYYC |
| | may be of any IgG | TIEDFYAMDYWGQGTTVTVSSASTKGPSVFPLAPSSKSTSGGTAALGC |
| | isotype) | LVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPELLGGPSV |
| | | FLFPPKPKDTLMISRTPEVTCVVVDVSHEDPEVKENWYVDGVEVHNAK |
| | | TKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEAL |
| | | HNHYTQKSLSLSPG |
| 27 | Heavy chain variable | QVQLVQSGAEVKKPGASVKVSCKASGYAFTSYWMHWVRQAPGQRLEWM |
| | region | GEIYPTNGGTTYNEKFKRRVTMTVDKSTSTAYMELSRLRSDDTAVYYC |
| | | TIEDFYAMDYWGQGTTVTVSS |
| 3 | HCDR1 (Kabat) | SYWMH |
| 4 | HCDR2 (Kabat) | EIYPTNGGTTYNEKFKR |
| 5 | HCDR3 (Kabat) | EDFYAMDY |
| 6 | HCDR1 (AbM) | GYAFTSYWMH |
| 7 | HCDR2 (AbM) | EIYPTNGGTT |
| 8 | HCDR3 (AbM) | EDFYAMDY |

Humanized anti-siglec-6 RND heavy chain 3b (hRND-H3b)

| | | |
|---|---|---|
| 28 | hRND-H3b, IgG1 | QVQLVQSGAEVKKPGASVKLSCKASGYAFTSYWMHWVRQAPGQRLEWI |
| | (Fc or constant region | GEIYPTNGGTTYNEKFKRRATLTVDKSTSTAYMELSRLRSDDTAVYYC |
| | may be of any IgG | TIEDFYAMDYWGQGTTVTVSSASTKGPSVFPLAPSSKSTSGGTAALGC |
| | isotype) | LVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPELLGGPSV |
| | | FLFPPKPKDTLMISRTPEVTCVVVDVSHEDPEVKENWYVDGVEVHNAK |
| | | TKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEAL |
| | | HNHYTQKSLSLSPG |
| 29 | Heavy chain variable | QVQLVQSGAEVKKPGASVKLSCKASGYAFTSYWMHWVRQAPGQRLEWI |
| | region | GETYPTNGGTTYNEKFKRRATLTVDKSTSTAYMELSRLRSDDTAVYYC |
| | | TIEDFYAMDYWGQGTTVTVSS |
| 3 | HCDR1 (Kabat) | SYWMH |
| 4 | HCDR2 (Kabat) | EIYPTNGGTTYNEKFKR |
| 5 | HCDR3 (Kabat) | EDFYAMDY |
| 6 | HCDR1 (AbM) | GYAFTSYWMH |
| 7 | HCDR2 (AbM) | EIYPTNGGTT |
| 8 | HCDR3 (AbM) | EDFYAMDY |

Humanized anti-siglec-6 RND light chain 1 (hRND-L1)

| | | |
|---|---|---|
| 30 | hRND-L1 | DIVMTQTPLSLSVTPGQPASISCKSSQSLLDSDGKTSLNWYLQKPGQS |
| | | PQLLIYLVSKLDSGVPDRFSGSGSGTDFTLKISRVEAEDVGVYYCWQG |
| | | THEPYTFGQGTKLEIKRTVAAPSVFIFPPSDEQLKSGTASVVCLLNNE |
| | | YPREAKVQWKVDNALQSGNSQESVTEQDSKDSTYSLSSTLTLSKADYE |
| | | KHKVYACEVTHQGLSSPVTKSENRGEC |
| 31 | Light chain variable | DIVMTQTPLSLSVTPGQPASISCKSSQSLLDSDGKTSLNWYLQKPGQS |
| | region | PQLLIYLVSKLDSGVPDRFSGSGSGTDFTLKISRVEAEDVGVYYCWQG |
| | | THEPYTFGQGTKLEIK |
| 32 | LCDR1 (Kabat) | KSSQSLLDSDGKTSLN |
| 12 | LCDR2 (Kabat) | LVSKLDS |
| 13 | LCDR3 (Kabat) | WQGTHEPYT |
| 34 | LCDR1 (AbM) | KSSQSLLDSDGKTSLN |
| 15 | LCDR2 (AbM) | LVSKLDS |
| 16 | LCDR3 (AbM) | WQGTHEPYT |

Humanized anti-siglec-6 RND light chain 2 (hRND-L2)

| | | |
|---|---|---|
| 35 | hRND-L2 | DIVMTQTPLSLSVTPGQPASISCKSSQSLLDSDGKTSLNWLLQKPGQS |
| | | PQLLIYLVSKLDSGVPDRFSGSGSGTDFTLKISRVEAEDVGVYYCWQG |
| | | THEPYTFGQGTKLEIKRTVAAPSVFIFPPSDEQLKSGTASVVCLLNNE |
| | | YPREAKVQWKVDNALQSGNSQESVTEQDSKDSTYSLSSTLTLSKADYE |
| | | KHKVYACEVTHQGLSSPVTKSENRGEC |
| 36 | Light chain variable | DIVMTQTPLSLSVTPGQPASISCKSSQSLLDSDGKTSLNWLLQKPGQS |
| | region | PQLLIYLVSKLDSGVPDRFSGSGSGTDFTLKISRVEAEDVGVYYCWQG |
| | | THEPYTFGQGTKLEIK |
| 32 | LCDR1 (Kabat) | KSSQSLLDSDGKTSLN |
| 12 | LCDR2 (Kabat) | LVSKLDS |
| 13 | LCDR3 (Kabat) | WQGTHFPYT |
| 34 | LCDR1 (AbM) | KSSQSLLDSDGKTSLN |
| 15 | LCDR2 (AbM) | LVSKLDS |
| 16 | LCDR3 (AbM) | WQGTHEPYT |

Humanized anti-siglec-6 RND light chain 3 (hRND-L3)

| | | |
|---|---|---|
| 37 | hRND-L3 | DIVMTQTPLSLSVTPGQPASISCKSSQSLLDSDGKTSLNWLLQKPGQS |
| | | PQRLIYLVSKLDSGVPDRFSGSGSGTDFTLKISRVEAEDVGVYYCWQG |
| | | THEPYTFGQGTKLEIKRTVAAPSVFIFPPSDEQLKSGTASVVCLLNNE |
| | | YPREAKVQWKVDNALQSGNSQESVTEQDSKDSTYSLSSTLTLSKADYE |
| | | KHKVYACEVTHQGLSSPVTKSENRGEC |
| 38 | Light chain variable | DIVMTQTPLSLSVTPGQPASISCKSSQSLLDSDGKTSLNWLLQKPGQS |
| | region | PQRLIYLVSKLDSGVPDRFSGSGSGTDFTLKISRVEAEDVGVYYCWQG |
| | | THEPYTFGQGTKLEIK |
| 32 | LCDR1 (Kabat) | KSSQSLLDSDGKTSLN |
| 12 | LCDR2 (Kabat) | LVSKLDS |
| 13 | LCDR3 (Kabat) | WQGTHEPYT |
| 34 | LCDR1 (AbM) | KSSQSLLDSDGKTSLN |
| 15 | LCDR2 (AbM) | LVSKLDS |
| 16 | LCDR3 (AbM) | WQGTHEPYT |

Human anti-siglec JML-1

| | | |
|---|---|---|
| 120 | Heavy chain, IgG1 | KVQLLESGGGLVQPGRSLRLSCAASGFTEDDYGMHWVRQAPGKGLEWV |
| | (Fc or constant region | SGISWNSGSIGYADSVKGRFTISRDNSKNTLYLQMNSLRAEDTAVYYC |
| | may be of any IgG | ARGGQTIDIWGQGTMVTVSSASTKGPSVFPLAPSSKSTSGGTAALGCL |
| | isotype) | VKDYFPEPVTVSWNSGALTSGVHTFPAVLQSSGLYSLSSVVTVPSSSL |
| | | GTQTYICNVNHKPSNTKVDKRVEPKSCDKTHTCPPCPAPELLGGPSVE |
| | | LFPPKPKDTLMISRTPEVTCVVVDVSHEDPEVKENWYVDGVEVHNAKT |
| | | KPREEQYNSTYRVVSVLTVLHQDWLNGKEYKCKVSNKALPAPIEKTIS |
| | | KAKGQPREPQVYTLPPSREEMTKNQVSLTCLVKGFYPSDIAVEWESNG |
| | | QPENNYKTTPPVLDSDGSFFLYSKLTVDKSRWQQGNVFSCSVMHEALH |
| | | NHYTQKSLSLSPG |
| 121 | Heavy chain variable | KVQLLESGGGLVQPGRSLRLSCAASGFTEDDYGMHWVRQAPGKGLEWV |
| | region | SGISWNSGSIGYADSVKGRFTISRDNSKNTLYLQMNSLRAEDTAVYYC |
| | | ARGGQTIDIWGQGTMVTVSS |
| 122 | HCDR1 (Kabat) | DYGMH |
| 123 | HCDR2 (Kabat) | GISWNSGSIGYADSVKG |
| 124 | HCDR3 (Kabat) | GGQTIDI |
| 125 | Light chain | DIQMTQSPSSLSASVGDRVTITCRASQSISSYLNWYQQKPGKAPKLLI |
| | | YAASSLQSGVPSRFSGSGSGTDFTLTISSLQPEDFATYYCQQSYSTPF |
| | | TFGPGTKVDIKRTVAAPSVFIFPPSDEQLKSGTASVVCLLNNFYPREA |
| | | KVQWKVDNALQSGNSQESVTEQDSKDSTYSLSSTLTLSKADYEKHKVY |
| | | ACEVTHQGLSSPVTKSENRGEC |
| 126 | Light chain variable | DIQMTQSPSSLSASVGDRVTITCRASQSISSYLNWYQQKPGKAPKLLI |
| | region | YAASSLQSGVPSRFSGSGSGTDFTLTISSLQPEDFATYYCQQSYSTPF |
| | | TFGPGTKVDIK |
| 127 | LCDR1 (Kabat) | RASQSISSYLN |
| 128 | LCDR2 (Kabat) | AASSLQS |
| 129 | LCDR3 (Kabat) | QQSYSTPFT |

Murine anti-siglec-6 M2 (mM2)

| | | |
|---|---|---|
| 132 | Heavy chain variable | EVQLQQSVAELVRPGASVKLSCTGSGENIENTYTHWVKQRPEQGLEWI |
| | region | GRIDPANTNTKYAPKFQGKATITADTSSNTAFLQLSSLTSEDTAIYYC |
| | | ALYDEDFWGPGTTLTVSS |
| 133 | HCDR1 (Kabat) | NTYTH |
| 134 | HCDR2 (Kabat) | RIDPANTNTKYAPKFQG |
| 135 | HCDR3 (Kabat) | YDEDF |
| 136 | Light chain variable | QIVLTQSPAIMSASPGEKVTMTCSASSSLTYMHWYQQKSGTSPKRWIY |
| | region | DTSELASGVPARFSGSGSGTSYSLTISSMEAEDAATYYCQQWNSDPPT |
| | | FGGGTKLEIK |
| 137 | LCDR1 (Kabat) | SASSSLTYMH |
| 138 | LCDR2 (Kabat) | DTSELAS |
| 139 | LCDR3 (Kabat) | QQWNSDPPT |

Murine anti-siglec-6 M7 (mM7)

| | | |
|---|---|---|
| 140 | Heavy chain variable | EFQLQQSGPELVKPGASVKISCKASDYSFTDYNMNWVKQSNGKSLEWI |
| | region | GVINPNYGTTSYNQKFKGKATLTVDQSSSTAYMQLNSLTSEDSAVYYC |
| | | ARSGGFSWGQGTLVTVSA |
| 141 | HCDR1 (Kabat) | DYNMN |
| 142 | HCDR2 (Kabat) | VINPNYGTTSYNQKFKG |
| 143 | HCDR3 (Kabat) | SGGFS |
| 144 | Light chain variable | DIVMTQSHTFMSTSVGDRVSITCKASQDVSTAVAWYQQKPGQSPKLLI |
| | region | FSASYRYTGVPDRFTGSGSGTDFTFTISSVQAEDLAVYYCQQHYSTPT |
| | | WTFGGGTKLEIK |
| 145 | LCDR1 (Kabat) | KASQDVSTAVA |
| 146 | LCDR2 (Kabat) | SASYRYT |
| 147 | LCDR3 (Kabat) | QQHYSTPTWT |

Murine anti-siglec-6 M11 (mM11)

| | | |
|---|---|---|
| 148 | Heavy chain variable | EVQLVESGGGLVQPKGSLKLSCAASGFTFKNYVMHWVRQTPGKGLEWV |
| | region | ARIRSKNSNYATYYADSVKDRFTISRDDSQSMLYLQMNNLKTEDTAMY |
| | | YCMRALVAYWGQGTLVTVSA |
| 149 | HCDR1 (Kabat) | NYVMH |
| 150 | HCDR2 (Kabat) | RIRSKNSNYATYYADSVKD |
| 151 | HCDR3 (Kabat) | ALVAY |
| 152 | Light chain variable | QLVLTQSPAIMSTSPGEKVTMTCSASSSVSYIYWYQQKPGSSPRLLIY |
| | region | DTSNLASGVPVRFSGSGSGTSYSLTISRMEAEDAATYYCQQWSSYPYT |
| | | FGGGTKLEIK |
| 153 | LCDR1 (Kabat) | SASSSVSYIY |
| 154 | LCDR2 (Kabat) | DTSNLAS |
| 155 | LCDR3 (Kabat) | QQWSSYPYT |

Chimeric anti-siglec-6 M11 (chM11)

| | | |
|---|---|---|
| 156 | Heavy chain | EVQLVESGGGLVQPKGSLKLSCAASGFTFKNYVMHWVRQTPGKGLEWV |
| | (Fc or constant region | ARIRSKNSNYATYYADSVKDRFTISRDDSQSMLYLQMNNLKTEDTAMY |
| | may be of any IgG | YCMRALVAYWGQGTLVTVSAASTKGPSVFPLAPSSKSTSGGTAALGCL |
| | isotype) | VKDYFPEPVTVSWNSGALTSGVHTFPAVLQSSGLYSLSSVVTVPSSSL |
| | | GTQTYICNVNHKPSNTKVDKKVEPKSCDKTHTCPPCPAPELLGGPSVE |
| | | LFPPKPKDTLMISRTPEVTCVVVDVSHEDPEVKENWYVDGVEVHNAKT |
| | | KPREEQYNSTYRVVSVLTVLHQDWLNGKEYKCKVSNKALPAPIEKTIS |
| | | KAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALH |
| | | NHYTQKSLSLSPGK |
| 157 | Light chain | QLVLTQSPAIMSTSPGEKVTMTCSASSSVSYIYWYQQKPGSSPRLLIY |
| | | DTSNLASGVPVRESGSGSGTSYSLTISRMEAEDAATYYCQQWSSYPYT |
| | | FGGGTKLEIKRTVAAPSVFIFPPSDEQLKSGTASVVCLLNNFYPREAK |
| | | VQWKVDNALQSGNSQESVTEQDSKDSTYSLSSTLTLSKADYEKHKVYA |
| | | CEVTHQGLSSPVTKSENRGEC |

Humanized anti-siglec-6 M11 heavy chain 1 (hM11-H1)

| | | |
|---|---|---|
| 158 | hM11-H1, IgG1 | EVQLVESGGGLVQPGGSLRLSCAASGFTFKNYVMHWVRQAPGKGLEWV |
| | (Fc or constant region | SRIRSKNSNYATYYADSVKGRFTISRDNSKNTLYLQMNSLRAEDTAVY |
| | may be of any IgG | YCAKALVAYWGQGTLVTVSSASTKGPSVFPLAPSSKSTSGGTAALGCL |
| | isotype) | VKDYFPEPVTVSWNSGALTSGVHTFPAVLQSSGLYSLSSVVTVPSSSL |
| | | GTQTYICNVNHKPSNTKVDKKVEPKSCDKTHTCPPCPAPELLGGPSVE |
| | | LFPPKPKDTLMISRTPEVTCVVVDVSHEDPEVKENWYVDGVEVHNAKT |
| | | KPREEQYNSTYRVVSVLTVLHQDWLNGKEYKCKVSNKALPAPIEKTIS |
| | | KAKGQPREPQVYTLPPSRDELTKNQVSLTCLVKGFYPSDIAVEWESNG |
| | | QPENNYKTTPPVLDSDGSFFLYSKLTVDKSRWQQGNVESCSVMHEALH |
| | | NHYTQKSLSLSPGK |
| 159 | Heavy chain variable | EVQLVESGGGLVQPGGSLRLSCAASGFTFKNYVMHWVRQAPGKGLEWV |
| | region | SRIRSKNSNYATYYADSVKGRFTISRDNSKNTLYLQMNSLRAEDTAVY |
| | | YCAKALVAYWGQGTLVTVSS |
| 149 | HCDR1 (Kabat) | NYVMH |
| 160 | HCDR2 (Kabat) | RIRSKNSNYATYYADSVKG |
| 151 | HCDR3 (Kabat) | ALVAY |

Humanized anti-siglec-6 M11 heavy chain 2 (hM11-H2)

| | | |
|---|---|---|
| 161 | hM11-H2, IgG1 | EVQLVESGGGLVQPGGSLRLSCAASGFTFKNYVMHWVRQAPGKGLEWV |
| | (Fc or constant region | ARIRSKNSNYATYYADSVKGRFTISRDDSKNTLYLQMNSLRAEDTAVY |
| | may be of any IgG | YCMRALVAYWGQGTLVTVSSASTKGPSVFPLAPSSKSTSGGTAALGCL |
| | isotype) | VKDYFPEPVTVSWNSGALTSGVHTFPAVLQSSGLYSLSSVVTVPSSSL |
| | | GTQTYICNVNHKPSNTKVDKKVEPKSCDKTHTCPPCPAPELLGGPSVE |
| | | LFPPKPKDTLMISRTPEVTCVVVDVSHEDPEVKENWYVDGVEVHNAKT |
| | | KPREEQYNSTYRVVSVLTVLHQDWLNGKEYKCKVSNKALPAPIEKTIS |
| | | KAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALH |
| | | NHYTQKSLSLSPGK |
| 162 | Heavy chain variable | EVQLVESGGGLVQPGGSLRLSCAASGFTFKNYVMHWVRQAPGKGLEWV |
| | region | ARIRSKNSNYATYYADSVKGRFTISRDDSKNTLYLQMNSLRAEDTAVY |
| | | YCMRALVAYWGQGTLVTVSS |
| 149 | HCDR1 (Kabat) | NYVMH |
| 160 | HCDR2 (Kabat) | RIRSKNSNYATYYADSVKG |
| 151 | HCDR3 (Kabat) | ALVAY |

Humanized anti-siglec-6 M11 light chain 1 (hM11-L1)

| | | |
|---|---|---|
| 163 | hM11-L1 | DIQMTQSPSSLSASVGDRVTITCRASSSVSYIYWYQQKPGKAPKLLIY |
| | | DTSNLASGVPSRFSGSGSGTDFTLTISSLQPEDFATYYCQQWSSYPYT |
| | | FGQGTKLEIKRTVAAPSVFIFPPSDEQLKSGTASVVCLLNNFYPREAK |
| | | VQWKVDNALQSGNSQESVTEQDSKDSTYSLSSTLTLSKADYEKHKVYA |
| | | CEVTHQGLSSPVTKSENRGEC |
| 164 | Light chain variable | DIQMTQSPSSLSASVGDRVTITCRASSSVSYIYWYQQKPGKAPKLLIY |
| | region | DTSNLASGVPSRFSGSGSGTDFTLTISSLQPEDFATYYCQQWSSYPYT |
| | | FGQGTKLEIK |
| 165 | LCDR1 (Kabat) | RASSSVSYIY |
| 154 | LCDR2 (Kabat) | DTSNLAS |
| 155 | LCDR3 (Kabat) | QQWSSYPYT |

Humanized anti-siglec-6 M11 light chain 2 (hM11-L2)

| | | |
|---|---|---|
| 166 | hM11-L2 | DIQMTQSPSSLSASVGDRVTITCRASSSVSYIYWYQQKPGKAPKLLIY |
| | | DTSNLASGVPSRFSGSGSGTDYTLTISSLQPEDFATYYCQQWSSYPYT |
| | | FGQGTKLEIKRTVAAPSVFIFPPSDEQLKSGTASVVCLLNNFYPREAK |
| | | VQWKVDNALQSGNSQESVTEQDSKDSTYSLSSTLTLSKADYEKHKVYA |
| | | CEVTHQGLSSPVTKSENRGEC |
| 167 | Light chain variable | DIQMTQSPSSLSASVGDRVTITCRASSSVSYIYWYQQKPGKAPKLLIY |
| | region | DTSNLASGVPSRFSGSGSGTDYTLTISSLQPEDFATYYCQQWSSYPYT |
| | | FGQGTKLEIK |
| 165 | LCDR1 (Kabat) | RASSSVSYIY |
| 154 | LCDR2 (Kabat) | DTSNLAS |
| 155 | LCDR3 (Kabat) | QQWSSYPYT |

Humanized anti-siglec-6 M11 light chain 3 (hM11-L3)

| | | |
|---|---|---|
| 168 | hM11-L3 | DIQLTQSPSSLSASVGDRVTMTCRASSSVSYIYWYQQKPGKAPKLLIY |
| | | DTSNLASGVPSRFSGSGSGTDYTLTISSMQPEDFATYYCQQWSSYPYT |
| | | FGQGTKLEIKRTVAAPSVFIFPPSDEQLKSGTASVVCLLNNFYPREAK |
| | | VQWKVDNALQSGNSQESVTEQDSKDSTYSLSSTLTLSKADYEKHKVYA |
| | | CEVTHQGLSSPVTKSENRGEC |
| 169 | Light chain variable | DIQLTQSPSSLSASVGDRVTMTCRASSSVSYIYWYQQKPGKAPKLLIY |
| | region | DTSNLASGVPSRFSGSGSGTDYTLTISSMQPEDFATYYCQQWSSYPYT |
| | | FGQGTKLEIK |
| 165 | LCDR1 (Kabat) | RASSSVSYIY |
| 154 | LCDR2 (Kabat) | DTSNLAS |
| 155 | LCDR3 (Kabat) | QQWSSYPYT |

TABLE 10
Sequences of exemplary antigen-binding sites capable of binding c-Kit

| | | |
|---|---|---|
| SEQ ID NO | Description | Sequence |

Murine anti-c-Kit SR1 (mSR1)

| | | |
|---|---|---|
| 39 | Heavy chain | QVQLQQPGAELVKPGASVKMSCKASGYTFTSYNMHWVKQTPGQGLEWI |
| | | GVIYSGNGDTSYNQKFKGKATLTADKSSSTAYMQINSLTSEDSAVYYC |
| | | ARERDTRFGNWGQGTLVTVSAAKTTAPSVYPLAPVCGDTTGSSVTLGC |
| | | LVKGYFPEPVTLTWNSGSLSSGVHTFPAVLQSDLYTLSSSVTVTSSTW |
| | | PSQSITCNVAHPASSTKVDKKIEPRGPTIKPCPPCKCPAPNLLGGPSV |
| | | FIFPPKIKDVLMISLSPIVTCVVVDVSEDDPDVQISWFVNNVEVHTAQ |
| | | TQTHREDYNSTLRVVSALPIQHQDWMSGKEFKCKVNNKDLPAPIERTI |
| | | SKPKGSVRAPQVYVLPPPEEEMTKKQVTLTCMVTDEMPEDIYVEWTNN |
| | | GKTELNYKNTEPVLDSDGSYFMYSKLRVEKKNWVERNSYSCSVVHEGL |
| | | HNHHTTKSFSRTPGK |
| 40 | Heavy chain variable | QVQLQQPGAELVKPGASVKMSCKASGYTFTSYNMHWVKQTPGQGLEWI |
| | region | GVIYSGNGDTSYNQKFKGKATLTADKSSSTAYMQINSLTSEDSAVYYC |
| | | ARERDTRFGNWGQGTLVTVSA |
| 41 | HCDR1 (Kabat) | SYNMH |
| 42 | HCDR2 (Kabat) | VIYSGNGDTSYNQKFKG |
| 43 | HCDR3 (Kabat) | ERDTRFGN |
| 44 | Light chain | NIVLTQSPASLAVSLGLRATISCRASESVDIYGNSFMHWYQQKPGQPP |
| | | KLLIYLASNLESGVPARFSGSGSRTDFTLTIDPVEADDAATYYCQQNN |
| | | EDPYTFGGGTKLEIKRADAAPTVSIFPPSSEQLTSGGASVVCFLNNFY |
| | | PKDINVKWKIDGSERQNGVLNSWTDQDSKDSTYSMSSTLTLTKDEYER |
| | | HNSYTCEATHKTSTSPIVKSFNRNEC |
| 45 | Light chain variable | NIVLTQSPASLAVSLGLRATISCRASESVDIYGNSFMHWYQQKPGQPP |
| | region | KLLIYLASNLESGVPARFSGSGSRTDFTLTIDPVEADDAATYYCQQNN |
| | | EDPYTFGGGTKLEIK |
| 46 | LCDR1 (Kabat) | RASESVDIYGNSFMH |
| 47 | LCDR2 (Kabat) | LASNLES |
| 48 | LCDR3 (Kabat) | QQNNEDPYT |

Chimeric anti-c-Kit SR1 (chSR1)

| | | |
|---|---|---|
| 49 | Heavy chain | QVQLQQPGAELVKPGASVKMSCKASGYTFTSYNMHWVKQTPGQGLEWI |
| | (Fc or constant region | GVIYSGNGDTSYNQKFKGKATLTADKSSSTAYMQINSLTSEDSAVYYC |
| | may be of any IgG | ARERDTRFGNWGQGTLVTVSAASTKGPSVFPLAPSSKSTSGGTAALGC |
| | isotype) | LVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPELLGGPSV |
| | | FLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEAL |
| | | HNHYTQKSLSLSPGK |
| 50 | Light chain | NIVLTQSPASLAVSLGLRATISCRASESVDIYGNSFMHWYQQKPGQPP |
| | | KLLIYLASNLESGVPARFSGSGSRTDFTLTIDPVEADDAATYYCQQNN |
| | | EDPYTFGGGTKLEIKRTVAAPSVFIFPPSDEQLKSGTASVVCLLNNFY |
| | | PREAKVQWKVDNALQSGNSQESVTEQDSKDSTYSLSSTLTLSKADYEK |
| | | HKVYACEVTHQGLSSPVTKSFNRGEC |

Humanized anti-c-Kit SR1 heavy chain 2 (hSR1-H2)

| | | |
|---|---|---|
| 51 | hSR1-H2, IgG1 | QVQLVQSGAEVKKPGASVKVSCKASGYTFTSYNMHWVRQAPGQGLEWM |
| | (Fc or constant region | GVIYSGNGDTSYAQKFQGRVTMTADTSTSTAYMELSSLRSEDTAVYYC |
| | may be of any IgG | ARERDTRFGNWGQGTLVTVSSASTKGPSVFPLAPSSKSTSGGTAALGC |
| | isotype) | LVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPELLGGPSV |
| | | FLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEAL |
| | | HNHYTQKSLSLSPGK |
| 52 | Heavy chain variable | QVQLVQSGAEVKKPGASVKVSCKASGYTFTSYNMHWVRQAPGQGLEWM |
| | region | GVIYSGNGDTSYAQKFQGRVTMTADTSTSTAYMELSSLRSEDTAVYYC |
| | | ARERDTRFGNWGQGTLVTVSS |
| 41 | HCDR1 (Kabat) | SYNMH |
| 53 | HCDR2 (Kabat) | VIYSGNGDTSYAQKFQG |
| 43 | HCDR3 (Kabat) | ERDTRFGN |

Humanized anti-c-Kit SR1 heavy chain 3 (hSR1-H3)

| | | |
|---|---|---|
| 54 | hSR1-H3, IgG1 | QVQLVQSGAEVKKPGASVKMSCKASGYTFTSYNMHWVRQAPGQGLEWI |
| | (Fc or constant region | GVIYSGNGDTSYAQKFQGRATLTADTSTSTAYMELSSLRSEDTAVYYC |
| | may be of any IgG | ARERDTRFGNWGQGTLVTVSSASTKGPSVFPLAPSSKSTSGGTAALGC |
| | isotype) | LVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPELLGGPSV |
| | | FLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESN |
| | | GQPENNYKTTPPVLDSDGSFFLYSKLTVDKSRWQQGNVESCSVMHEAL |
| | | HNHYTQKSLSLSPGK |
| 55 | Heavy chain variable | QVQLVQSGAEVKKPGASVKMSCKASGYTFTSYNMHWVRQAPGQGLEWI |
| | region | GVIYSGNGDTSYAQKFQGRATLTADTSTSTAYMELSSLRSEDTAVYYC |
| | | ARERDTRFGNWGQGTLVTVSS |
| 41 | HCDR1 (Kabat) | SYNMH |
| 53 | HCDR2 (Kabat) | VIYSGNGDTSYAQKFQG |
| 43 | HCDR3 (Kabat) | ERDTRFGN |

Humanized anti-c-Kit SR1 heavy chain 2b (hSR1-H2b)

| | | |
|---|---|---|
| 56 | hSR1-H2b, IgG1 | QVQLVQSGAEVKKPGASVKVSCKASGYTFTSYNMHWVRQAPGQGLEWM |
| | (Fc or constant region | GVIYSGNGDTSYAQKFQGRVTMTADKSTSTAYMELSSLRSEDTAVYYC |
| | may be of any IgG | ARERDTRFGNWGQGTLVTVSSASTKGPSVFPLAPSSKSTSGGTAALGC |
| | isotype) | LVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPELLGGPSV |
| | | FLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEAL |
| | | HNHYTQKSLSLSPGK |
| 57 | Heavy chain variable | QVQLVQSGAEVKKPGASVKVSCKASGYTFTSYNMHWVRQAPGQGLEWM |
| | region | GVIYSGNGDTSYAQKFQGRVTMTADKSTSTAYMELSSLRSEDTAVYYC |
| | | ARERDTRFGNWGQGTLVTVSS |
| 41 | HCDR1 (Kabat) | SYNMH |
| 53 | HCDR2 (Kabat) | VIYSGNGDTSYAQKFQG |
| 43 | HCDR3 (Kabat) | ERDTRFGN |

Humanized anti-c-Kit SR1 heavy chain 3b (hSR1-H3b)

| | | |
|---|---|---|
| 58 | hSR1-H3b, IgG1 | QVQLVQSGAEVKKPGASVKMSCKASGYTFTSYNMHWVRQAPGQGLEWI |
| | (Fc or constant region | GVIYSGNGDTSYAQKFQGRATLTADKSTSTAYMELSSLRSEDTAVYYC |
| | may be of any IgG | ARERDTRFGNWGQGTLVTVSSASTKGPSVFPLAPSSKSTSGGTAALGC |
| | isotype) | LVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPELLGGPSV |
| | | FLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEAL |
| | | HNHYTQKSLSLSPGK |
| 59 | Heavy chain variable | QVQLVQSGAEVKKPGASVKMSCKASGYTFTSYNMHWVRQAPGQGLEWI |
| | region | GVIYSGNGDTSYAQKFQGRATLTADKSTSTAYMELSSLRSEDTAVYYC |
| | | ARERDTRFGNWGQGTLVTVSS |
| 41 | HCDR1 (Kabat) | SYNMH |
| 53 | HCDR2 (Kabat) | VIYSGNGDTSYAQKFQG |
| 43 | HCDR3 (Kabat) | ERDTRFGN |

hSR1-H2 anti-c-Kit variants

| | | |
|---|---|---|
| 60 | HCDR1 (Kabat) variant 1 | SYTMH |
| 61 | HCDR1 (Kabat) variant 2 | SYQMH |
| 62 | HCDR1 (Kabat) variant 3 | NYNMH |
| 63 | HCDR2 (Kabat) variant 1 | VIYSGSGDTSYAQKFQG |
| 64 | HCDR2 (Kabat) variant 2 | VIYSGQGDTSYAQKFQG |
| 65 | HCDR2 (Kabat) variant 3 | VIYSGNGSTSYAQKFQG |
| 66 | HCDR2 (Kabat) variant 4 | VIYSGNGETSYAQKFQG |
| 67 | HCDR2 (Kabat) variant 5 | VIFSGNGDTSYAQKFQG |
| 68 | HCDR2 (Kabat) variant 6 | VIHSGNGDTSYAQKFQG |
| 69 | HCDR3 (Kabat) variant 1 | ERETRFGN |
| 70 | HCDR3 (Kabat) variant 2 | ERSTRFGN |
| 71 | HCDR3 (Kabat) variant 3 | EKDTRFGN |
| 72 | HCDR3 (Kabat) variant 4 | ERDTKFGN |
| 73 | hSR1-H2 N33T variable | QVQLVQSGAEVKKPGASVKVSCKASGYTFTSYTMHWVRQAPGQGLEWM |
| | region | GVIYSGNGDTSYAQKFQGRVTMTADTSTSTAYMELSSLRSEDTAVYYC |
| | | ARERDTRFGNWGQGTLVTVSS |
| 74 | hSR1-H2 N33Q variable | QVQLVQSGAEVKKPGASVKVSCKASGYTFTSYQMHWVRQAPGQGLEWM |
| | region | GVIYSGNGDTSYAQKFQGRVTMTADTSTSTAYMELSSLRSEDTAVYYC |
| | | ARERDTRFGNWGQGTLVTVSS |
| 75 | hSR1-H2 N3IN variable | QVQLVQSGAEVKKPGASVKVSCKASGYTFTNYNMHWVRQAPGQGLEWM |
| | region | GVIYSGNGDTSYAQKFQGRVTMTADTSTSTAYMELSSLRSEDTAVYYC |
| | | ARERDTRFGNWGQGTLVTVSS |
| 76 | hSR1-H2 N54Svariable | QVQLVQSGAEVKKPGASVKVSCKASGYTFTSYNMHWVRQAPGQGLEWM |
| | region | GVIYSGSGDTSYAQKFQGRVTMTADTSTSTAYMELSSLRSEDTAVYYC |
| | | ARERDTRFGNWGQGTLVTVSS |
| 77 | hSR1-H2 N54Q variable | QVQLVQSGAEVKKPGASVKVSCKASGYTFTSYNMHWVRQAPGQGLEWM |
| | region | GVIYSGQGDTSYAQKFQGRVTMTADTSTSTAYMELSSLRSEDTAVYYC |
| | | ARERDTRFGNWGQGTLVTVSS |
| 78 | hSR1-H2 N56S variable | QVQLVQSGAEVKKPGASVKVSCKASGYTFTSYNMHWVRQAPGQGLEWM |
| | region | GVIYSGNGSTSYAQKFQGRVTMTADTSTSTAYMELSSLRSEDTAVYYC |
| | | ARERDTRFGNWGQGTLVTVSS |
| 79 | hSR1-H2 N56E variable | QVQLVQSGAEVKKPGASVKVSCKASGYTFTSYNMHWVRQAPGQGLEWM |
| | region | GVIYSGNGETSYAQKFQGRVTMTADTSTSTAYMELSSLRSEDTAVYYC |
| | | ARERDTRFGNWGQGTLVTVSS |
| 80 | hSR1-H2 N52F variable | QVQLVQSGAEVKKPGASVKVSCKASGYTFTSYNMHWVRQAPGQGLEWM |
| | region | GVIFSGNGDTSYAQKFQGRVTMTADTSTSTAYMELSSLRSEDTAVYYC |
| | | ARERDTRFGNWGQGTLVTVSS |
| 81 | hSR1-H2 N52H variable | QVQLVQSGAEVKKPGASVKVSCKASGYTFTSYNMHWVRQAPGQGLEWM |
| | region | GVIHSGNGDTSYAQKFQGRVTMTADTSTSTAYMELSSLRSEDTAVYYC |
| | | ARERDTRFGNWGQGTLVTVSS |
| 82 | hSR1-H2 N97E variable | QVQLVQSGAEVKKPGASVKVSCKASGYTFTSYNMHWVRQAPGQGLEWM |
| | region | GVIYSGNGDTSYAQKFQGRVTMTADTSTSTAYMELSSLRSEDTAVYYC |
| | | ARERETREGNWGQGTLVTVSS |
| 83 | hSR1-H2 N97S ariable | QVQLVQSGAEVKKPGASVKVSCKASGYTFTSYNMHWVRQAPGQGLEWM |
| | region | GVIYSGNGDTSYAQKFQGRVTMTADTSTSTAYMELSSLRSEDTAVYYC |
| | | ARERSTRFGNWGQGTLVTVSS |
| 84 | hSR1-H2 N96K variable | QVQLVQSGAEVKKPGASVKVSCKASGYTFTSYNMHWVRQAPGQGLEWM |
| | region | GVIYSGNGDTSYAQKFQGRVTMTADTSTSTAYMELSSLRSEDTAVYYC |
| | | AREKDTRFGNWGQGTLVTVSS |
| 85 | hSR1-H2 N99K variable | QVQLVQSGAEVKKPGASVKVSCKASGYTFTSYNMHWVRQAPGQGLEWM |
| | region | GVIYSGNGDTSYAQKFQGRVTMTADTSTSTAYMELSSLRSEDTAVYYC |
| | | ARERDTKFGNWGQGTLVTVSS |

Humanized anti-c-Kit SR1 light chain 2 (hSR1-L2)

| | | |
|---|---|---|
| 86 | hSR1-L2 | DIQLTQSPSSLSASVGDRATITCRASESVDIYGNSFMHWYQQKPGKAP |
| | | KLLIYLASNLESGVPSRFSGSGSGTDFTLTISSVQPEDFATYYCQQNN |
| | | EDPYTFGQGTKLEIKRTVAAPSVFIFPPSDEQLKSGTASVVCLLNNFY |
| | | PREAKVQWKVDNALQSGNSQESVTEQDSKDSTYSLSSTLTLSKADYEK |
| | | HKVYACEVTHQGLSSPVTKSFNRGEC |
| 87 | Light chain variable | DIQLTQSPSSLSASVGDRATITCRASESVDIYGNSFMHWYQQKPGKAP |
| | region | KLLIYLASNLESGVPSRFSGSGSGTDFTLTISSVQPEDFATYYCQQNN |
| | | EDPYTFGQGTKLEIK |
| 46 | LCDR1 (Kabat) | RASESVDIYGNSFMH |
| 47 | LCDR2 (Kabat) | LASNLES |
| 48 | LCDR3 (Kabat) | QQNNEDPYT |

Humanized anti-c-Kit SR1 light chain 2r (hSR1-L2r)

| | | |
|---|---|---|
| 88 | hSR1-L2r | DIQLTQSPSSLSASVGDRATITCRASESVDIYGNSFMHWYQQKPGKAP |
| | | KLLIYLASNLESGVPSRFSGSGSRTDFTLTISSVQPEDFATYYCQQNN |
| | | EDPYTFGQGTKLEIKRTVAAPSVFIFPPSDEQLKSGTASVVCLLNNFY |
| | | PREAKVQWKVDNALQSGNSQESVTEQDSKDSTYSLSSTLTLSKADYEK |
| | | HKVYACEVTHQGLSSPVTKSFNRGEC |
| 89 | Light chain variable | DIQLTQSPSSLSASVGDRATITCRASESVDIYGNSFMHWYQQKPGKAP |
| | region | KLLIYLASNLESGVPSRFSGSGSRTDFTLTISSVQPEDFATYYCQQNN |
| | | EDPYTFGQGTKLEIK |
| 46 | LCDR1 (Kabat) | RASESVDIYGNSFMH |
| 47 | LCDR2 (Kabat) | LASNLES |
| 48 | LCDR3 (Kabat) | QQNNEDPYT |

hSR1-L2 variants

| | | |
|---|---|---|
| 90 | LCDR1 (Kabat) variant 1 | RASESVDSYGNSFMH |
| 91 | LCDR1 (Kabat) variant 2 | RASESVDNYGNSFMH |
| 92 | LCDR1 (Kabat) variant 3 | RASESVDIFGNSFMH |
| 93 | LCDR1 (Kabat) variant 4 | RASESVDIHGNSFMH |
| 94 | LCDR1 (Kabat) variant 5 | RASESVDIYGSSFMH |
| 95 | LCDR1 (Kabat) variant 6 | RASESVDIYGQSFMH |
| 96 | LCDR2 (Kabat) variant 1 | AASNLES |
| 97 | LCDR2 (Kabat) variant 2 | LASSLES |
| 98 | LCDR3 (Kabat) variant 1 | QQNQEDPYT |
| 99 | LCDR3 (Kabat) variant 2 | QQNSEDPYT |
| 100 | LCDR3 (Kabat) variant 3 | QQNNDDPYT |
| 101 | LCDR3 (Kabat) variant 4 | QQNNSDPYT |
| 102 | LCDR3 (Kabat) variant 5 | QQNNEEPYT |
| 103 | LCDR3 (Kabat) variant 6 | QQNNESPYT |
| 104 | LCDR3 (Kabat) variant 7 | QQNNEDPFT |
| 105 | hSR1-L2 I30aS variable | DIQLTQSPSSLSASVGDRATITCRASESVDSYGNSFMHWYQQKP |
| | region | GKAPKLLIYLASNLESGVPSRFSGSGSGTDFTLTISSVQPEDFA |
| | | TYYCQQNNEDPYTFGQGTKLEIK |
| 106 | hSR1-L2 I30aN variable | DIQLTQSPSSLSASVGDRATITCRASESVDNYGNSFMHWYQQKPGKAP |
| | region | KLLIYLASNLESGVPSRFSGSGSGTDFTLTISSVQPEDFATYYCQQNN |
| | | EDPYTFGQGTKLEIK |
| 107 | hSR1-L2 Y30bF variable | DIQLTQSPSSLSASVGDRATITCRASESVDIFGNSFMHWYQQKPGKAP |
| | region | KLLIYLASNLESGVPSRFSGSGSGTDFTLTISSVQPEDFATYYCQQNN |
| | | EDPYTFGQGTKLEIK |
| 108 | hSR1-L2 Y30bH variable | DIQLTQSPSSLSASVGDRATITCRASESVDIHGNSFMHWYQQKPGKAP |
| | region | KLLIYLASNLESGVPSRFSGSGSGTDFTLTISSVQPEDFATYYCQQNN |
| | | EDPYTFGQGTKLEIK |
| 109 | hSR1-L2 N30dS variable | DIQLTQSPSSLSASVGDRATITCRASESVDIYGSSFMHWYQQKPGKAP |
| | region | KLLIYLASNLESGVPSRFSGSGSGTDFTLTISSVQPEDFATYYCQQNN |
| | | EDPYTFGQGTKLEIK |
| 110 | hSR1-L2 N30dQ variable | DIQLTQSPSSLSASVGDRATITCRASESVDIYGQSEMHWYQQKPGKAP |
| | region | KLLIYLASNLESGVPSRESGSGSGTDFTLTISSVQPEDFATYYCQQNN |
| | | EDPYTFGQGTKLEIK |
| 111 | hSR1-L2 L50A variable | DIQLTQSPSSLSASVGDRATITCRASESVDIYGNSFMHWYQQKP |
| | region | GKAPKLLIYAASNLESGVPSRESGSGSGTDFTLTISSVQPEDFA |
| | | TYYCQQNNEDPYTFGQGTKLEIK |
| 112 | hSR1-L2 N53S variable | DIQLTQSPSSLSASVGDRATITCRASESVDIYGNSFMHWYQQKPGKAP |
| | region | KLLIYLASSLESGVPSRFSGSGSGTDFTLTISSVQPEDFATYYCQQNN |
| | | EDPYTFGQGTKLEIK |
| 113 | hSR1-L2 N92Q variable | DIQLTQSPSSLSASVGDRATITCRASESVDIYGNSFMHWYQQKPGKAP |
| | region | KLLIYLASNLESGVPSRFSGSGSGTDFTLTISSVQPEDFATYYCQQNQ |
| | | EDPYTFGQGTKLEIK |
| 114 | hSR1-L2 N92S variable | DIQLTQSPSSLSASVGDRATITCRASESVDIYGNSFMHWYQQKPGKAP |
| | region | KLLIYLASNLESGVPSRFSGSGSGTDFTLTISSVQPEDFATYYCQQNS |
| | | EDPYTFGQGTKLEIK |
| 115 | hSR1-L2 E93D variable | DIQLTQSPSSLSASVGDRATITCRASESVDIYGNSEMHWYQQKPGKAP |
| | region | KLLIYLASNLESGVPSRESGSGSGTDFTLTISSVQPEDFATYYCQQNN |
| | | DDPYTFGQGTKLEIK |
| 116 | hSR1-L2 E93S variable | DIQLTQSPSSLSASVGDRATITCRASESVDIYGNSFMHWYQQKPGKAP |
| | region | KLLIYLASNLESGVPSRESGSGSGTDETLTISSVQPEDFATYYCQQNN |
| | | SDPYTFGQGTKLEIK |
| 117 | hSR1-L2 D94E variable | DIQLTQSPSSLSASVGDRATITCRASESVDIYGNSFMHWYQQKPGKAP |
| | region | KLLIYLASNLESGVPSRFSGSGSGTDFTLTISSVQPEDFATYYCQQNN |
| | | EEPYTFGQGTKLEIK |
| 118 | hSR1-L2 D94S variable | DIQLTQSPSSLSASVGDRATITCRASESVDIYGNSFMHWYQQKPGKAP |
| | region | KLLIYLASNLESGVPSRFSGSGSGTDFTLTISSVQPEDFATYYCQQNN |
| | | ESPYTFGQGTKLEIK |
| 119 | hSR1-L2 Y96F variable | DIQLTQSPSSLSASVGDRATITCRASESVDIYGNSFMHWYQQKPGKAP |
| | region | KLLIYLASNLESGVPSRFSGSGSGTDFTLTISSVQPEDFATYYCQQNN |
| | | EDPFTFGQGTKLEIK |

### INCORPORATION BY REFERENCE

TABLE 11
Other sequences

| SEQ | | |
|---|---|---|
| ID | | |
| NO | Description | Sequence |
| 130 | Human immunoglobulin | ASTKGPSVFPLAPSSKST |
| | G1 (IgG1) heavy chain | SGGTAALGCLVKDYFPEP |
| | constant (CH) region | VTVSWNSGALTSGVHTFP |
| | | AVLQSSGLYSLSSVVTVP |
| | | SSSLGTQTYICNVNHKPS |
| | | NTKVDKKVEPKSCDKTHT |
| | | CPPCPAPELLGGPSVELF |
| | | PPKPKDTLMISRTPEVTC |
| | | VVVDVSHEDPEVKENWYV |
| | | DGVEVHNAKTKPREEQYN |
| | | STYRVVSVLTVLHQDWLN |
| | | GKEYKCKVSNKALPAPIE |
| | | KTISKAKGQPREPQVYTL |
| | | PPSRDELTKNQVSLTCLV |
| | | KGFYPSDIAVEWESNGQP |
| | | ENNYKTTPPVLDSDGSFF |
| | | LYSKLTVDKSRWQQGNVF |
| | | SCSVMHEALHNHYTQKSL |
| | | SLSPGK |
| 131 | Human immunoglobulin | RTVAAPSVFIFPPSDEQL |
| | kappa constant region | KSGTASVVCLLNNFYPRE |
| | | AKVQWKVDNALQSGNSQE |
| | | SVTEQDSKDSTYSLSSTL |
| | | TLSKADYEKHKVYACEVT |
| | | HQGLSSPVTKSENRGEC |

Unless stated to the contrary, the entire disclosure of each of the patent documents and scientific articles referred to herein is incorporated by reference for all purposes.

### EQUIVALENTS/OTHER EMBODIMENTS

Those skilled in the art will recognize or be able to ascertain using no more than routine experimentation, many equivalents to the specific embodiments described herein. Such equivalents are intended to be encompassed by the following claims.

What is claimed is:

1. An antibody or antigen-binding fragment thereof, comprising a first antigen-binding domain that is capable of binding siglec-6 and a second antigen-binding domain that is capable of binding c-Kit.

2. The antibody or antigen-binding fragment thereof of claim 1, wherein the first antigen-binding domain is capable of binding siglec-6 with a higher affinity than the second antigen-binding domain is capable of binding c-Kit.

3. The antibody or antigen-binding fragment thereof of claim 1, wherein the first antigen-binding domain is capable of binding siglec-6 with an affinity characterized by a K_{D} value that is lower than about 100 uM, lower than about 100 nM, lower than about 10 nM, lower than about 1 nM, lower than about 100 pM, lower than about 10 pM, lower than about 1 pM, lower than about 100 fM, or lower than about 10 fM.

4. The antibody or antigen-binding fragment thereof of claim 3, wherein the first antigen-binding domain is capable of binding c-Kit with an affinity characterized by a K_{D} value that is between about 1 nM and about 900 uM, between about 5 nM and about 500 uM, between about 10 nM and about 100 uM, between about 20 nM and about 10 uM, or between about 25 and about 500 nM.

5. The antibody or antigen-binding fragment thereof of any one of claims 1-4, wherein the antibody or antigen-binding fragment is capable of binding a cell expressing siglect-6.

6. The antibody or antigen-binding fragment thereof of any one of claims 1-5, wherein the antibody or antigen-binding fragment is capable of binding a mast cell.

7. The antibody or antigen-binding fragment thereof of claim 6, wherein the antibody or antigen-binding fragment binds a mast cell preferentially than another immune cell.

8. The antibody or antigen-binding fragment thereof of claim 6, wherein the immune cell is T cell, dendritic cell, macrophage, neutrophil, or basophil.

9. The antibody or antigen-binding fragment thereof of any one of claims 1-8, wherein the antibody or antigen-binding fragment thereof does not induce mast cell degranulation.

10. The antibody or antigen-binding fragment thereof of any one of claims 1-9, wherein the antibody or antigen-binding fragment thereof has a comparable or lower internalization rate compared to a reference antibody capable of binding siglect-6.

11. The antibody or antigen-binding fragment thereof of claim 10, wherein the reference antibody capable of binding siglect-6 comprises a heavy chain variable (VH) region comprising the amino acid sequence of SEQ ID NO: 2, and a light chain variable (VL) region comprising the amino acid sequence of SEQ ID NO: 10.

12. The antibody or antigen-binding fragment thereof of any one of claims 1-11, wherein the antibody or antigen-binding fragment thereof has a comparable or lower toxicity compared to a reference antibody capable of binding c-Kit.

13. The antibody or antigen-binding fragment thereof of claim 12, wherein the reference antibody capable of binding c-Kit comprises a VH region comprising the amino acid sequence of SEQ ID NO: 40, and a VL region comprising the amino acid sequence of SEQ ID NO: 45.

14. The antibody or antigen-binding fragment thereof of any one of claims 1-13, wherein the first antigen-binding domain comprises:
(a) a VH region comprising:
heavy chain complementarity-determining region 1 (HCDR1) having the amino acid sequence of SEQ ID NO: 3, or a sequence differing in 1 or 2 amino acids therefrom,
HCDR2 having the amino acid sequence of SEQ ID NO: 4 or 21, or a sequence differing in 1 or 2 amino acids therefrom, and
HCDR3 having the amino acid sequence of SEQ ID NO: 5, or a sequence differing in 1 or 2 amino acids therefrom; and
(b) a VL region comprising:
heavy chain complementarity-determining region 1 (LCDR1) having the amino acid sequence of SEQ ID NO: 11 or 32, or a sequence differing in 1 or 2 amino acids therefrom,
LCDR2 having the amino acid sequence of SEQ ID NO: 12, or a sequence differing in 1 or 2 amino acids therefrom, and
LCDR3 having the amino acid sequence of SEQ ID NO: 13, or a sequence differing in 1 or 2 amino acids therefrom.

15. The antibody or antigen-binding fragment thereof of claim 14, wherein the first antigen-binding domain comprises:
(a) a VH region comprising:
HCDR1 having the amino acid sequence of SEQ ID NO: 3,
HCDR2 having the amino acid sequence of SEQ ID NO: 4 or 21, and
HCDR3 having the amino acid sequence of SEQ ID NO: 5; and
(b) a VL region comprising:
LCDR1 having the amino acid sequence of SEQ ID NO: 11 or 32,
LCDR2 having the amino acid sequence of SEQ ID NO: 12, and
LCDR3 having the amino acid sequence of SEQ ID NO: 13.

16. The antibody or antigen-binding fragment thereof of claim 14, wherein the first antigen-binding domain comprises:
(a) a VH region comprising:
(i) HCDR1, HCDR2, and HCDR3 having the amino acid sequence of SEQ ID NOs: 3, 4, and 5, respectively; or
(ii) HCDR1, HCDR2, and HCDR3 having the amino acid sequence of SEQ ID NOs: 3, 21, and 5, respectively; and
(b) a VL region comprising:
(i) LCDR1, LCDR2, and LCDR3 having the amino acid sequence of SEQ ID NOs: 11, 12, and 13, respectively; or
(ii) LCDR1, LCDR2, and LCDR3 having the amino acid sequence of SEQ ID NOs: 32, 12, and 13, respectively.

17. The antibody or antigen-binding fragment thereof of any one of claims 14-16, wherein the first antigen-binding domain comprises:
(a) a VH region comprising an amino acid sequence having at least 70%, at least 80%, at least 90%, or at least 95% sequence identity to the amino acid sequence of SEQ ID NO: 2, 20, 23, 25, 27, or 29; and
(b) a VL region comprising an amino acid sequence having at least 70%, at least 80%, at least 90%, or at least 95% sequence identity to the amino acid sequence of SEQ ID NO: 10, 31, 36, or 38.

18. The antibody or antigen-binding fragment thereof of claim 17, wherein the first antigen-binding domain comprises:
(a) a VH region having the amino acid sequence of SEQ ID NO: 2, 20, 23, 25, 27, or 29; and
(b) a VL region having the amino acid sequence of SEQ ID NO: 10, 31, 36, or 38.

19. The antibody or antigen-binding fragment thereof of claim 17 or 18, wherein the first antigen-binding domain comprises:
(a) a VH region having the amino acid sequence of SEQ ID NO: 2, and a VL region having the amino acid sequence of SEQ ID NO: 10;
(b) a VH region having the amino acid sequence of SEQ ID NO: 23, and a VL region having the amino acid sequence of SEQ ID NO: 38;
(c) a VH region having the amino acid sequence of SEQ ID NO: 25, and a VL region having the amino acid sequence of SEQ ID NO: 38;
(d) a VH region having the amino acid sequence of SEQ ID NO: 27, and a VL region having the amino acid sequence of SEQ ID NO: 38; or
(e) a VH region having the amino acid sequence of SEQ ID NO: 29, and a VL region having the amino acid sequence of SEQ ID NO: 38.

20. The antibody or antigen-binding fragment thereof of claim 19, wherein the first antigen-binding domain comprises:
a VH region having the amino acid sequence of SEQ ID NO: 25, and a VL region having the amino acid sequence of SEQ ID NO: 38.

21. The antibody or antigen-binding fragment thereof of any one of claims 1-13, wherein the first antigen-binding domain comprises:
(a) a VH region comprising:
HCDR1 having the amino acid sequence of SEQ ID NO: 122, or a sequence differing in 1 or 2 amino acids therefrom,
HCDR2 having the amino acid sequence of SEQ ID NO: 123, or a sequence differing in 1 or 2 amino acids therefrom, and
HCDR3 having the amino acid sequence of SEQ ID NO: 124, or a sequence differing in 1 or 2 amino acids therefrom; and
(b) a VL region comprising:
LCDR1 having the amino acid sequence of SEQ ID NO: 127, or a sequence differing in 1 or 2 amino acids therefrom,
LCDR2 having the amino acid sequence of SEQ ID NO: 128, or a sequence differing in 1 or 2 amino acids therefrom, and
LCDR3 having the amino acid sequence of SEQ ID NO: 129, or a sequence differing in 1 or 2 amino acids therefrom.

22. The antibody or antigen-binding fragment thereof of claim 21, wherein the first antigen-binding domain comprises:
(a) a VH region comprising:
HCDR1 having the amino acid sequence of SEQ ID NO: 122,
HCDR2 having the amino acid sequence of SEQ ID NO: 123, and
HCDR3 having the amino acid sequence of SEQ ID NO: 124; and
(b) a VL region comprising:
LCDR1 having the amino acid sequence of SEQ ID NO: 127,
LCDR2 having the amino acid sequence of SEQ ID NO: 128, and
LCDR3 having the amino acid sequence of SEQ ID NO: 139.

23. The antibody or antigen-binding fragment thereof of claim 21 or 22, wherein the first antigen-binding domain comprises:
(a) a VH region comprising an amino acid sequence having at least 70%, at least 80%, at least 90%, or at least 95% sequence identity to the amino acid sequence of SEQ ID NO: 121; and
(b) a VL region comprising an amino acid sequence having at least 70%, at least 80%, at least 90%, or at least 95% sequence identity to the amino acid sequence of SEQ ID NO: 126.

24. The antibody or antigen-binding fragment thereof of claim 23, wherein the first antigen-binding domain comprises:
(a) a VH region having the amino acid sequence of SEQ ID NO: 121; and
(b) a VL region having the amino acid sequence of SEQ ID NO: 126.

25. The antibody or antigen-binding fragment thereof of any one of claims 1-13, wherein the first antigen-binding domain comprises:
(a) a VH region comprising:
HCDR1 having the amino acid sequence of SEQ ID NO: 133, 141, or 149, or a sequence differing in 1 or 2 amino acids therefrom,
HCDR2 having the amino acid sequence of SEQ ID NO: 134, 142, 150, or 160, or a sequence differing in 1 or 2 amino acids therefrom, and
HCDR3 having the amino acid sequence of SEQ ID NO: 135, 143, or 151, or a sequence differing in 1 or 2 amino acids therefrom; and
(b) a VL region comprising:
LCDR1 having the amino acid sequence of SEQ ID NO: 137, 145, 153, or 165, or a sequence differing in 1 or 2 amino acids therefrom,
LCDR2 having the amino acid sequence of SEQ ID NO: 138, 146, or 154, or a sequence differing in 1 or 2 amino acids therefrom, and
LCDR3 having the amino acid sequence of SEQ ID NO: 139, 147, or 155, or a sequence differing in 1 or 2 amino acids therefrom.

26. The antibody or antigen-binding fragment thereof of claim 25, wherein the first antigen-binding domain comprises:
(a) a VH region comprising:
HCDR1 having the amino acid sequence of SEQ ID NO: 133, 141, or 149,
HCDR2 having the amino acid sequence of SEQ ID NO: 134, 142, 150, or 160, and
HCDR3 having the amino acid sequence of SEQ ID NO: 135, 143, or 151; and
(b) a VL region comprising:
LCDR1 having the amino acid sequence of SEQ ID NO: 137, 145, 153, or 165,
LCDR2 having the amino acid sequence of SEQ ID NO: 138, 146, or 154, and
LCDR3 having the amino acid sequence of SEQ ID NO: 139, 147, or 155.

27. The antibody or antigen-binding fragment thereof of claim 25 or 26, wherein the first antigen-binding domain comprises:
(a) a VH region comprising:
(i) HCDR1, HCDR2, and HCDR3 having the amino acid sequence of SEQ ID NOs: 133, 134, and 135, respectively;
(ii) HCDR1, HCDR2, and HCDR3 having the amino acid sequence of SEQ ID NOs: 141, 142, and 143, respectively;
(iii) HCDR1, HCDR2, and HCDR3 having the amino acid sequence of SEQ ID NOs: 149, 150, and 151, respectively; or
(iv) HCDR1, HCDR2, and HCDR3 having the amino acid sequence of SEQ ID NOs: 149, 160, and 151, respectively; and
(b) a VL region comprising:
(i) LCDR1, LCDR2, and LCDR3 having the amino acid sequence of SEQ ID NOs: 137, 138, and 139, respectively;
(ii) LCDR1, LCDR2, and LCDR3 having the amino acid sequence of SEQ ID NOs: 145, 146, and 147, respectively;
(iii) LCDR1, LCDR2, and LCDR3 having the amino acid sequence of SEQ ID NOs: 153, 154, and 155, respectively; or
(iv) LCDR1, LCDR2, and LCDR3 having the amino acid sequence of SEQ ID NOs: 165, 154, and 155, respectively.

28. The antibody or antigen-binding fragment thereof of any one of claims 25-27, wherein the first antigen-binding domain comprises:
(a) a VH region comprising an amino acid sequence having at least 70%, at least 80%, at least 90%, or at least 95% sequence identity to the amino acid sequence of SEQ ID NO: 132, 140, 148, 159, or 162; and
(b) a VL region comprising an amino acid sequence having at least 70%, at least 80%, at least 90%, or at least 95% sequence identity to the amino acid sequence of SEQ ID NO: 136, 144, 152, 164, 167, or 169.

29. The antibody or antigen-binding fragment thereof of claim 28, wherein the first antigen-binding domain comprises:
(a) a VH region having the amino acid sequence of SEQ ID NO: 132, 140, 148, 159, or 162; and
(b) a VL region having the amino acid sequence of SEQ ID NO: 136, 144, 152, 164, 167, or 169.

30. The antibody or antigen-binding fragment thereof of claim 28 or 29, wherein the first antigen-binding domain comprises:
(a) a VH region having the amino acid sequence of SEQ ID NO: 132, and a VL region having the amino acid sequence of SEQ ID NO: 139;
(b) a VH region having the amino acid sequence of SEQ ID NO: 140, and a VL region having the amino acid sequence of SEQ ID NO: 144; or
(c) a VH region having the amino acid sequence of SEQ ID NO: 148, and a VL region having the amino acid sequence of SEQ ID NO: 152.

31. The antibody or antigen-binding fragment thereof of claim 28 or 29, wherein the first antigen-binding domain comprises:
(a) a VH region having the amino acid sequence of SEQ ID NO: 159, and a VL region having the amino acid sequence of SEQ ID NO: 164;
(6) a VH region having the amino acid sequence of SEQ ID NO: 162, and a VL region having the amino acid sequence of SEQ ID NO: 167; or
(c) a VH region having the amino acid sequence of SEQ ID NO: 162, and a VL region having the amino acid sequence of SEQ ID NO: 169.

32. The antibody or antigen-binding fragment thereof of any one of claims 1-31, wherein the second antigen-binding domain comprises:
(a) a VH region comprising:
HCDR1 having the amino acid sequence of SEQ ID NO: 41, or a sequence differing in 1 or 2 amino acids therefrom,
HCDR2 having the amino acid sequence of SEQ ID NO: 42 or 53, or a sequence differing in 1 or 2 amino acids therefrom, and
HCDR3 having the amino acid sequence of SEQ ID NO: 43, or a sequence differing in 1 or 2 amino acids therefrom; and
(b) a VL region comprising:
LCDR1 having the amino acid sequence of SEQ ID NO: 46, or a sequence differing in 1 or 2 amino acids therefrom,
LCDR2 having the amino acid sequence of SEQ ID NO: 47, or a sequence differing in 1 or 2 amino acids therefrom, and
LCDR3 having the amino acid sequence of SEQ ID NO: 48, or a sequence differing in 1 or 2 amino acids therefrom.

33. The antibody or antigen-binding fragment thereof of claim 32, wherein the second antigen-binding domain comprises:
(a) a VH region comprising:
HCDR1 having the amino acid sequence of SEQ ID NO: 41,
HCDR2 having the amino acid sequence of SEQ ID NO: 42 or 53, and
HCDR3 having the amino acid sequence of SEQ ID NO: 43; and
(b) a VL region comprising:
LCDR1 having the amino acid sequence of SEQ ID NO: 46,
LCDR2 having the amino acid sequence of SEQ ID NO: 47, and
LCDR3 having the amino acid sequence of SEQ ID NO: 48.

34. The antibody or antigen-binding fragment thereof of claim 32, wherein the second antigen-binding domain comprises a VH region comprising:
(a) HCDR1 having the amino acid sequence of SEQ ID NO: 60, 61, or 62,
(b) HCDR2 having the amino acid sequence of SEQ ID NO: 63, 64, 65, 66, 67, or 68, and
(c) HCDR3 having the amino acid sequence of SEQ ID NO: 69, 70, 71, or 72.

35. The antibody or antigen-binding fragment thereof of claim 32, wherein the second antigen-binding domain comprises a VH region comprising:
(a) LCDR1 having the amino acid sequence of SEQ ID NO: 90, 91, 92, 93, 94, or 95,
(b) LCDR2 having the amino acid sequence of SEQ ID NO: 96 or 97, and
(c) LCDR3 having the amino acid sequence of SEQ ID NO: 98, 99, 100, 101, 102, 103, or 104.

36. The antibody or antigen-binding fragment thereof of any one of claims 32-35, wherein the second antigen-binding domain comprises:
(a) a VH region comprising:
(i) HCDR1, HCDR2, and HCDR3 having the amino acid sequence of SEQ ID NOs: 41, 42, and 43, respectively;
(ii) HCDR1, HCDR2, and HCDR3 having the amino acid sequence of SEQ ID NOs: 41, 53, and 43, respectively;
(iii) HCDR1, HCDR2, and HCDR3 having the amino acid sequence of SEQ ID NOs: 60, 53, and 43, respectively;
(iv) HCDR1, HCDR2, and HCDR3 having the amino acid sequence of SEQ ID NOs: 61, 53, and 43, respectively;
(v) HCDR1, HCDR2, and HCDR3 having the amino acid sequence of SEQ ID NOs: 62, 53, and 43, respectively;
(vi) HCDR1, HCDR2, and HCDR3 having the amino acid sequence of SEQ ID NOs: 41, 63, and 43, respectively;
(vii) HCDR1, HCDR2, and HCDR3 having the amino acid sequence of SEQ ID NOs: 41, 64, and 43, respectively;
(viii) HCDR1, HCDR2, and HCDR3 having the amino acid sequence of SEQ ID NOs: 41, 65, and 43, respectively;
(ix) HCDR1, HCDR2, and HCDR3 having the amino acid sequence of SEQ ID NOs: 41, 66, and 43, respectively;
(x) HCDR1, HCDR2, and HCDR3 having the amino acid sequence of SEQ ID NOs: 41, 67, and 43, respectively;
(xi) HCDR1, HCDR2, and HCDR3 having the amino acid sequence of SEQ ID NOs: 41, 68, and 43, respectively;
(xii) HCDR1, HCDR2, and HCDR3 having the amino acid sequence of SEQ ID NOs: 41, 53, and 69, respectively;
(xiii) HCDR1, HCDR2, and HCDR3 having the amino acid sequence of SEQ ID NOs: 41, 53, and 70, respectively;
(xiv) HCDR1, HCDR2, and HCDR3 having the amino acid sequence of SEQ ID NOs: 41, 53, and 71, respectively; or
(xv) HCDR1, HCDR2, and HCDR3 having the amino acid sequence of SEQ ID NOs: 41, 53, and 72, respectively; and
(b) a VL region comprising:
(i) LCDR1, LCDR2, and LCDR3 having the amino acid sequence of SEQ ID NOs: 46, 47, and 48, respectively;
(ii) LCDR1, LCDR2, and LCDR3 having the amino acid sequence of SEQ ID NOs: 90, 47, and 48, respectively;
(iii) LCDR1, LCDR2, and LCDR3 having the amino acid sequence of SEQ ID NOs: 91, 47, and 48, respectively;
(iv) LCDR1, LCDR2, and LCDR3 having the amino acid sequence of SEQ ID NOs: 92, 47, and 48, respectively;
(v) LCDR1, LCDR2, and LCDR3 having the amino acid sequence of SEQ ID NOs: 93, 47, and 48, respectively;
(vi) LCDR1, LCDR2, and LCDR3 having the amino acid sequence of SEQ ID NOs: 94, 47, and 48, respectively;
(vii) LCDR1, LCDR2, and LCDR3 having the amino acid sequence of SEQ ID NOs: 95, 47, and 48, respectively;
(viii) LCDR1, LCDR2, and LCDR3 having the amino acid sequence of SEQ ID NOs: 46, 96, and 48, respectively;
(ix) LCDR1, LCDR2, and LCDR3 having the amino acid sequence of SEQ ID NOs: 46, 97, and 48, respectively;
(x) LCDR1, LCDR2, and LCDR3 having the amino acid sequence of SEQ ID NOs: 46, 47, and 98, respectively;
(xi) LCDR1, LCDR2, and LCDR3 having the amino acid sequence of SEQ ID NOs: 46, 47, and 99, respectively;
(xii) LCDR1, LCDR2, and LCDR3 having the amino acid sequence of SEQ ID NOs: 46, 47, and 100, respectively;
(xiii) LCDR1, LCDR2, and LCDR3 having the amino acid sequence of SEQ ID NOs: 46, 47, and 101, respectively;
(xiv) LCDR1, LCDR2, and LCDR3 having the amino acid sequence of SEQ ID NOs: 46, 47, and 102, respectively;
(xv) LCDR1, LCDR2, and LCDR3 having the amino acid sequence of SEQ ID NOs: 46, 47, and 103, respectively; or
(xvi) LCDR1, LCDR2, and LCDR3 having the amino acid sequence of SEQ ID NOs: 46, 47, and 104, respectively.

37. The antibody or antigen-binding fragment thereof of any one of claims 32-36, wherein the second antigen-binding domain comprises:
(a) a VH region comprising an amino acid sequence having at least 70%, at least 80%, at least 90%, or at least 95% sequence identity to the amino acid sequence of SEQ ID NO: 40, 52, 55, 57, 59, 73, 74, 75, 76, 77, 78, 79, 80, 81, 82, 83, 84, or 85; and
(b) a VL region comprising an amino acid sequence having at least 70%, at least 80%, at least 90%, or at least 95% sequence identity to the amino acid sequence of SEQ ID NO: 45, 87, 89, 105, 106, 107, 108, 109, 110, 111, 112, 113, 114, 115, 116, 117, 118, or 119.

38. The antibody or antigen-binding fragment thereof of claim 37, wherein the second antigen-binding domain comprises:
(a) a VH region having the amino acid sequence of SEQ ID NO: 40, 52, 55, 57, 59, 73, 74, 75, 76, 77, 78, 79, 80, 81, 82, 83, 84, or 85; and
(b) a VL region having the amino acid sequence of SEQ ID NO: 45, 87, 89, 105, 106, 107, 108, 109, 110, 111, 112, 113, 114, 115, 116, 117, 118, or 119.

39. The antibody or antigen-binding fragment thereof of claim 37 or 38, wherein the second antigen-binding domain comprises:
(a) a VH region having the amino acid sequence of SEQ ID NO: 40, and a VL region having the amino acid sequence of SEQ ID NO: 45;
(b) a VH region having the amino acid sequence of SEQ ID NO: 52, and a VL region having the amino acid sequence of SEQ ID NO: 87;
(c) a VH region having the amino acid sequence of SEQ ID NO: 55, and a VL region having the amino acid sequence of SEQ ID NO: 87;
(d) a VH region having the amino acid sequence of SEQ ID NO: 57, and a VL region having the amino acid sequence of SEQ ID NO: 87;
(e) a VH region having the amino acid sequence of SEQ ID NO: 59, and a VL region having the amino acid sequence of SEQ ID NO: 87;
(f) a VH region having the amino acid sequence of SEQ ID NO: 52, and a VL region having the amino acid sequence of SEQ ID NO: 89; or
(g) a VH region having the amino acid sequence of SEQ ID NO: 55, and a VL region having the amino acid sequence of SEQ ID NO: 89.

40. The antibody or antigen-binding fragment thereof of claim 39, wherein the second antigen-binding domain comprises:
a VH region having the amino acid sequence of SEQ ID NO: 52, and a VL region having the amino acid sequence of SEQ ID NO: 87.

41. The antibody or antigen-binding fragment thereof of any one of the claims 1-40, wherein the antibody or antigen-binding fragment thereof comprises:
(a) a first heavy chain, comprising the VH region of the first antigen-binding domain, and a first heavy chain constant (CH) region or a fragment thereof;
(b) a first light chain, comprising the VL region of the first antigen-binding domain, and a first light chain constant (CL) region or a fragment thereof;
(c) a second heavy chain, comprising the VH region of the second antigen-binding domain, and a second CH region or a fragment thereof, and
(d) a second light chain, comprising the VL region of the second antigen-binding domain, and a second CL region or a fragment thereof.

42. The antibody or antigen-binding fragment thereof of claim 41, wherein the first and second CH region each comprise an amino acid sequence having at least 70%, at least 80%, at least 90%, or at least 95% sequence identity to the amino acid sequence of SEQ ID NO: 130.

43. The antibody or antigen-binding fragment thereof of claim 34 or 35, wherein the first and second heavy chain form a heterodimer, optionally wherein one of the first and second heavy chains comprises a Serine residue at position 366, an Alanine residue at position 368 and a Valine residue at position 407; and the other heavy chain comprises a Tryptophan residue at position 366, wherein the numbering of the constant region is as per the EU index.

44. The antibody or antigen-binding fragment thereof of any one of claims 41-43, wherein the first and/or second CH region comprise one or more mutations that increase serum half-life of the antibody or antigen-binding fragment.

45. The antibody or antigen-binding fragment thereof of claim 44, wherein the first and/or second CH region comprises is a human IgG1 CH comprising the following mutations: M252Y, S254T, and T256E, wherein the numbering of the constant region is as per the EU index.

46. The antibody or antigen-binding fragment thereof of any one of claims 43-45, wherein the first and/or second CH region comprise one or more mutations that reduce or abrogate effector function.

47. The antibody or antigen-binding fragment thereof of claim 46, wherein the first and/or second CH region is a human IgG1 CH comprising one of the following mutation(s), wherein the numbering of the constant region is as per the EU index:
(a) L234A and/or L235A;
(b) A327G, A330S, and/or P331S;
(c) E233P, L234V, L235A, and/or G236del;
(d) E233P, L234V, and/or L235A;
(e) E233P, L234V, L235A, G236del, A327G, A330S, and/or P331S;
(f) E233P, L234V, L235A, A327G, A330S, and/or P331S;
(g) N297A;
(h) N297G;
(i) N297Q;
(j) L242C, N297C, and/or K334C;
(k) A287C, N297G, and/or L306C;
(l) R292C, N297G, and/or V302C;
(m) N297G, V323C, and/or I332C;
(n) V259C, N297G, and/or L306C;
(o) L234F, L235Q, K322Q, M252Y, S254T, and/or T256E;
(p) L234A, L235A, and/or P329G; or
(q) L234A, L235Q, and K322Q.

48. The antibody or antigen-binding fragment thereof of claim 46, wherein the first and/or second CH region is a human IgG2 CH comprising one of the following mutation(s), wherein the numbering of the constant region is as per the EU index:
(a) A330S and/or P331S;
(b) V234A, G237A, P238S, H268A, V309L, A330S, and/or P331S; or
(c) V234A, G237A, H268Q, V309L, A330S, P331S, C232S, C233S, S267E, L328F, M252Y, S254T, and/or T256E.

49. The antibody or antigen-binding fragment thereof of claim 46, wherein the first and/or second CH region is a human IgG4 CH comprising one of the following mutation(s), wherein the numbering of the constant region is as per the EU index:
(a) E233P, F234V, L235A, and/or G236del;
(b) E233P, F234V, and/or L235A;
(c) S228P and/or L235E; or
(d) S228P and/or L235A.

50. The antibody or antigen-binding fragment thereof of any one of claims 41-45, wherein the first and/or second CH region comprise one or more mutations that enhance effector function.

51. The antibody or antigen-binding fragment thereof of claim 50, wherein the first and/or second CH region is a human IgG1 CH comprising one of the following mutation(s), wherein the numbering of the constant region is as per the EU index:
(a) F243L, R292P, Y300L, V305I, and/or P396L;
(b) S239D and/or 1332E;
(c) S239D, 1332E, and/or A330L;
(d) S298A, E333A, and/or K334A;
(e) G236A, S239D, and/or 1332E;
(f) K326W and/or E333S;
(g) S267E, H268F, and/or S324T; or
(h) E345R, E430G, and/or S440Y.

52. The antibody or antigen-binding fragment thereof of any one of claims 43-51, wherein at least one of the first and second heavy chains is non-fucosylated.

53. The antibody or antigen-binding fragment thereof of claim 50, wherein the antibody or antigen-binding fragment thereof is produced in a cell line
(a) having an alpha-1,6-fucosyltransferase (Fut8) knockout; or
(b) line overexpressing β1,4-N-acetylglucosaminyltransferase III (GnT-III) and optionally overexpressing Golgi μ-mannosidase II (ManII).

54. The antibody or antigen-binding fragment thereof of any one of claims 43-53, wherein the first and/or second CL region is a human kappa constant region.

55. The antibody or antigen-binding fragment thereof of any one of claims 43-54, wherein:
(a) (i) the first antigen-binding domain comprises:
a VH region comprising HCDR1, HCDR2, and HCDR3 having the amino acid sequence of SEQ ID NOs: 3, 21, and 5, respectively; and
a VL region comprising LCDR1, LCDR2, and LCDR3 having the amino acid sequence of SEQ ID NOs: 32, 12, and 13, respectively; and
(ii) the second antigen-binding domain comprises:
a VH region comprising HCDR1, HCDR2, and HCDR3 having the amino acid sequence of SEQ ID NOs: 41, 53, and 43, respectively; and
a VL region comprising LCDR1, LCDR2, and LCDR3 having the amino acid sequence of SEQ ID NOs: 46, 47, and 48, respectively; or
(b) (i) the first antigen-binding domain comprises:
a VH region comprising HCDR1, HCDR2, and HCDR3 having the amino acid sequence of SEQ ID NOs: 122, 123, and 124, respectively; and
a VL region comprising LCDR1, LCDR2, and LCDR3 having the amino acid sequence of SEQ ID NOs: 127, 128, and 129, respectively; and
(ii) the second antigen-binding domain comprises:
a VH region comprising HCDR1, HCDR2, and HCDR3 having the amino acid sequence of SEQ ID NOs: 41, 53, and 43, respectively; and
a VL region comprising LCDR1, LCDR2, and LCDR3 having the amino acid sequence of SEQ ID NOs: 46, 47, and 48, respectively.

56. The antibody or antigen-binding fragment thereof of claim 55, wherein:
(a) the first antigen-binding domain comprises a VH region having the amino acid sequence of SEQ ID NO: 25, and a VL region having the amino acid sequence of SEQ ID NO: 38; and
the second antigen-binding domain comprises a VH region having the amino acid sequence of SEQ ID NO: 52, and a VL region having the amino acid sequence of SEQ ID NO: 87; or
(b) the first antigen-binding domain comprises a VH region having the amino acid sequence of SEQ ID NO: 121, and a VL region having the amino acid sequence of SEQ ID NO: 126; and
the second antigen-binding domain comprises a VH region having the amino acid sequence of SEQ ID NO: 52, and a VL region having the amino acid sequence of SEQ ID NO: 87.

57. The antibody or antigen-binding fragment thereof of any one of claims 1-56, wherein the antigen-binding fragment is a Fab, a Fab′, a Fab2, a F(ab′)2, Fv, a single-chain Fv (scFv), or a diabody.

58. The antibody or antigen-binding fragment thereof of any one of claims 1-57, wherein the antibody or antigen-binding fragment thereof is conjugated to an agent.

59. The antibody or antigen-binding fragment thereof of claim 58, wherein the agent is a cytotoxic agent or label.

60. A composition comprising the antibody or antigen-binding fragment thereof of any one of claims 1-59.

61. The composition of claim 60, wherein the antibody or antigen-binding fragment comprises an Fc region and N-glycoside-linked carbohydrate chains linked to the Fc region, optionally wherein less than 50% of the N-glycoside-linked carbohydrate chains contain a fucose residue.

62. The composition of claim 61, wherein substantially none of the N-glycoside-linked carbohydrate chains contain a fucose residue.

63. An antibody or antigen binding fragment thereof that is capable of binding to siglec-6, comprising:
(a) a heavy chain variable (VH) region comprising:
heavy chain complementarity-determining region 1 (HCDR1) having the amino acid sequence of SEQ ID NO: 133, 141, or 149, or a sequence differing in 1 or 2 amino acids therefrom,
HCDR2 having the amino acid sequence of SEQ ID NO: 134, 142, 150, or 160, or a sequence differing in 1 or 2 amino acids therefrom, and
HCDR3 having the amino acid sequence of SEQ ID NO: 135, 143, or 151, or a sequence differing in 1 or 2 amino acids therefrom; and
(b) a light chain variable (VL) region comprising:
light chain complementarity-determining region 1 (LCDR1) having the amino acid sequence of SEQ ID NO: 137, 145, 153, or 165, or a sequence differing in 1 or 2 amino acids therefrom,
LCDR2 having the amino acid sequence of SEQ ID NO: 138, 146, or 154, or a sequence differing in 1 or 2 amino acids therefrom, and
LCDR3 having the amino acid sequence of SEQ ID NO: 139, 147, or 155, or a sequence differing in 1 or 2 amino acids therefrom.

64. The antibody or antigen binding fragment thereof of claim 63, comprising:
(a) a VH region comprising:
HCDR1 having the amino acid sequence of SEQ ID NO: 133, 141, or 149,
HCDR2 having the amino acid sequence of SEQ ID NO: 134, 142, 150, or 160, and
HCDR3 having the amino acid sequence of SEQ ID NO: 135, 143, or 151; and
(b) a VL region comprising:
LCDR1 having the amino acid sequence of SEQ ID NO: 137, 145, 153, or 165,
LCDR2 having the amino acid sequence of SEQ ID NO: 138, 146, or 154, and
LCDR3 having the amino acid sequence of SEQ ID NO: 139, 147, or 155.

65. The antibody or antigen binding fragment thereof of claim 63, comprising:
(a) (i) a VH region comprising:
HCDR1 having the amino acid sequence of SEQ ID NO: 133, or a sequence differing in 1 or 2 amino acids therefrom,
HCDR2 having the amino acid sequence of SEQ ID NO: 134, or a sequence differing in 1 or 2 amino acids therefrom, and
HCDR3 having the amino acid sequence of SEQ ID NO: 135, or a sequence differing in 1 or 2 amino acids therefrom; and
(ii) a VL region comprising:
LCDR1 having the amino acid sequence of SEQ ID NO: 137, or a sequence differing in 1 or 2 amino acids therefrom,
LCDR2 having the amino acid sequence of SEQ ID NO: 138, or a sequence differing in 1 or 2 amino acids therefrom, and
LCDR3 having the amino acid sequence of SEQ ID NO: 139, or a sequence differing in 1 or 2 amino acids therefrom; or
(b) (i) a VH region comprising:
HCDR1 having the amino acid sequence of SEQ ID NO: 141, or a sequence differing in 1 or 2 amino acids therefrom,
HCDR2 having the amino acid sequence of SEQ ID NO: 142, or a sequence differing in 1 or 2 amino acids therefrom, and
HCDR3 having the amino acid sequence of SEQ ID NO: 143, or a sequence differing in 1 or 2 amino acids therefrom; and
(ii) a VL region comprising:
LCDR1 having the amino acid sequence of SEQ ID NO: 145, or a sequence differing in 1 or 2 amino acids therefrom,
LCDR2 having the amino acid sequence of SEQ ID NO: 146, or a sequence differing in 1 or 2 amino acids therefrom, and
LCDR3 having the amino acid sequence of SEQ ID NO: 147, or a sequence differing in 1 or 2 amino acids therefrom; or
(c) (i) a VH region comprising:
HCDR1 having the amino acid sequence of SEQ ID NO: 149, or a sequence differing in 1 or 2 amino acids therefrom,
HCDR2 having the amino acid sequence of SEQ ID NO: 150 or 160, or a sequence differing in 1 or 2 amino acids therefrom, and
HCDR3 having the amino acid sequence of SEQ ID NO: 151, or a sequence differing in 1 or 2 amino acids therefrom; and
(ii) a VL region comprising:
LCDR1 having the amino acid sequence of SEQ ID NO: 153 or 165, or a sequence differing in 1 or 2 amino acids therefrom,
LCDR2 having the amino acid sequence of SEQ ID NO: 154, or a sequence differing in 1 or 2 amino acids therefrom, and
LCDR3 having the amino acid sequence of SEQ ID NO: 155, or a sequence differing in 1 or 2 amino acids therefrom.

66. The antibody or antigen binding fragment thereof of claim 65, comprising:
(a) (i) a VH region comprising:
HCDR1 having the amino acid sequence of SEQ ID NO: 133,
HCDR2 having the amino acid sequence of SEQ ID NO: 134, and
HCDR3 having the amino acid sequence of SEQ ID NO: 135; and
(ii) a VL region comprising:
LCDR1 having the amino acid sequence of SEQ ID NO: 137,
LCDR2 having the amino acid sequence of SEQ ID NO: 138, and
LCDR3 having the amino acid sequence of SEQ ID NO: 139; or
(b) (i) a VH region comprising:
HCDR1 having the amino acid sequence of SEQ ID NO: 141,
HCDR2 having the amino acid sequence of SEQ ID NO: 142, and
HCDR3 having the amino acid sequence of SEQ ID NO: 143; and
(ii) a VL region comprising:
LCDR1 having the amino acid sequence of SEQ ID NO: 145,
LCDR2 having the amino acid sequence of SEQ ID NO: 146, and
LCDR3 having the amino acid sequence of SEQ ID NO: 147; or
(c) (i) a VH region comprising:
HCDR1 having the amino acid sequence of SEQ ID NO: 149,
HCDR2 having the amino acid sequence of SEQ ID NO: 150 or 160, and
HCDR3 having the amino acid sequence of SEQ ID NO: 151; and
(ii) a VL region comprising:
LCDR1 having the amino acid sequence of SEQ ID NO: 153 or 165,
LCDR2 having the amino acid sequence of SEQ ID NO: 154, and
LCDR3 having the amino acid sequence of SEQ ID NO: 155.

67. The antibody or antigen binding fragment thereof of any one of claims 63-66, comprising:
(a) a VH region comprising:
(i) HCDR1, HCDR2, and HCDR3 having the amino acid sequence of SEQ ID NOs: 133, 134, and 135, respectively;
(ii) HCDR1, HCDR2, and HCDR3 having the amino acid sequence of SEQ ID NOs: 141, 142, and 143, respectively;
(iii) HCDR1, HCDR2, and HCDR3 having the amino acid sequence of SEQ ID NOs: 149, 150, and 151, respectively; or
(iv) HCDR1, HCDR2, and HCDR3 having the amino acid sequence of SEQ ID NOs: 149, 160, and 151, respectively; and
(b) a VL region comprising:
(i) LCDR1, LCDR2, and LCDR3 having the amino acid sequence of SEQ ID NOs: 137, 138, and 139, respectively;
(ii) LCDR1, LCDR2, and LCDR3 having the amino acid sequence of SEQ ID NOs: 145, 146, and 147, respectively;
(iii) LCDR1, LCDR2, and LCDR3 having the amino acid sequence of SEQ ID NOs: 153, 154, and 155, respectively; or
(iv) LCDR1, LCDR2, and LCDR3 having the amino acid sequence of SEQ ID NOs: 165, 154, and 155, respectively.

68. The antibody or antigen binding fragment thereof of any one of claims 63-67, wherein
the VH region comprises an amino acid sequence having at least 70%, at least 80%, at least 90%, or at least 95% sequence identity to the amino acid sequence of SEQ ID NO: 132, 140, 148, 159, or 162; and
the VL region comprises an amino acid sequence having at least 70%, at least 80%, at least 90%, or at least 95% sequence identity to the amino acid sequence of SEQ ID NO: 136, 144, 152, 164, 167, or 169.

69. The antibody or antigen binding fragment thereof of any one of claims 63-68, wherein
the VH region comprises the amino acid sequence of SEQ ID NO: 132, 140, 148, 159, or 162; and
the VL region comprises the amino acid sequence of SEQ ID NO: 136, 144, 152, 164, 167, or 169.

70. The antibody or antigen-binding fragment thereof of any one of claims 63-69, wherein
(a) the VH region comprises the amino acid sequence of SEQ ID NO: 132, and the VL region comprises the amino acid sequence of SEQ ID NO: 139;
(b) the VH region comprises the amino acid sequence of SEQ ID NO: 140, and the VL region comprises the amino acid sequence of SEQ ID NO: 144; or
(c) the VH region comprises the amino acid sequence of SEQ ID NO: 148, and the VL region comprises the amino acid sequence of SEQ ID NO: 152.

71. The antibody or antigen-binding fragment thereof of any one of claims 63-69, wherein
(a) the VH region comprises the amino acid sequence of SEQ ID NO: 159, and the VL region comprises the amino acid sequence of SEQ ID NO: 164;
(6) the VH region comprises the amino acid sequence of SEQ ID NO: 162, and the VL region comprises the amino acid sequence of SEQ ID NO: 167; or
(c) the VH region comprises the amino acid sequence of SEQ ID NO: 162, and the VL region comprises the amino acid sequence of SEQ ID NO: 169.

72. The antibody or antigen-binding fragment thereof of any one of claims 63-71, wherein the antibody is a humanized antibody.

73. A humanized antibody or antigen binding fragment thereof that is capable of binding to siglec-6, comprising:
(a) a heavy chain variable domain (VH) comprising:
heavy chain complementarity-determining region 1 (HCDR1) having the amino acid sequence of SEQ ID NO: 3,
HCDR2 having the amino acid sequence of SEQ ID NO: 21, and
HCDR3 having the amino acid sequence of SEQ ID NO: 5; and
(a) a light chain variable domain (VL) comprising:
light chain complementarity-determining region 1 (LCDR1) having the amino acid sequence of SEQ ID NO: 32,
LCDR2 having the amino acid sequence of SEQ ID NO: 12, and
LCDR3 having the amino acid sequence of SEQ ID NO: 13.

74. The antibody or antigen binding fragment thereof of claim 73, wherein
the VH region comprises an amino acid sequence having at least 70%, at least 80%, at least 90%, or at least 95% sequence identity to the amino acid sequence of SEQ ID NO: 20, 23, 25, 27, or 29; and
the VL region comprises an amino acid sequence having at least 70%, at least 80%, at least 90%, or at least 95% sequence identity to the amino acid sequence of SEQ ID NO: 31, 36, or 38.

75. The antibody or antigen-binding fragment thereof of claim 73 or 74, wherein
the VH region comprises the amino acid sequence of SEQ ID NO: 20, 23, 25, 27, or 29; and
the VL region comprises the amino acid sequence of SEQ ID NO: 31, 36, or 38.

76. The antibody or antigen-binding fragment thereof of any one of claims 73-75, wherein
(a) the VH region comprises the amino acid sequence of SEQ ID NO: 23, and the VL region comprises the amino acid sequence of SEQ ID NO: 38;
(b) the VH region comprises the amino acid sequence of SEQ ID NO: 25, and the VL region comprises the amino acid sequence of SEQ ID NO: 38;
(c) the VH region comprises the amino acid sequence of SEQ ID NO: 27, and the VL region comprises the amino acid sequence of SEQ ID NO: 38; or
(d) the VH region comprises the amino acid sequence of SEQ ID NO: 29, and the VL region comprises the amino acid sequence of SEQ ID NO: 38.

77. The antibody or antigen-binding fragment thereof of claim 76, wherein the VH region comprises the amino acid sequence of SEQ ID NO: 25, and the VL region comprises the amino acid sequence of SEQ ID NO: 38.

78. The antibody or antigen binding fragment thereof of any one of claims 63-77, wherein the antibody or antigen binding fragment thereof is capable of binding siglec-6 with a K_{D} value of at most about 10 nM, at most about 5 nM, or at most about 1 nM.

79. The antibody or antigen binding fragment thereof of any one of claims 63-78, wherein the antibody or antigen binding fragment thereof
(a) is capable of binding human siglec-6;
(b) is capable of binding cynomolgus siglec-6;
(c) is capable of binding a mast cell, optionally preferentially than another immune cell (e.g., T cell, dendritic cell, macrophage, neutrophil, or basophil);
(d) does not induce mast cell degranulation; and/or
(e) has a comparable or lower internalization rate compared to a reference antibody capable of binding siglect-6, optionally wherein the reference antibody comprises a VH region comprising the amino acid sequence of SEQ ID NO: 2 and a VL region comprising the amino acid sequence of SEQ ID NO: 10.

80. A humanized antibody or antigen binding fragment thereof that is capable of binding to c-Kit, comprising:
(a) a heavy chain variable domain (VH) comprising:
heavy chain complementarity-determining region 1 (HCDR1) having the amino acid sequence of SEQ ID NO: 41, or a sequence differing in 1 amino acid therefrom,
HCDR2 having the amino acid sequence of SEQ ID NO: 53, or a sequence differing in 1 amino acid therefrom, and
HCDR3 having the amino acid sequence of SEQ ID NO: 43, or a sequence differing in 1 amino acid therefrom; and
(a) a light chain variable domain (VL) comprising:
light chain complementarity-determining region 1 (LCDR1) having the amino acid sequence of SEQ ID NO: 46, or a sequence differing in 1 amino acid therefrom,
LCDR2 having the amino acid sequence of SEQ ID NO: 47, or a sequence differing in 1 amino acid therefrom, and
LCDR3 having the amino acid sequence of SEQ ID NO: 48, or a sequence differing in 1 amino acid therefrom.

81. The antibody or antigen binding fragment thereof of claim 80, comprising
(a) a VH region comprising:
HCDR1 having the amino acid sequence of SEQ ID NO: 41,
HCDR2 having the amino acid sequence of SEQ ID NO: 53, and
HCDR3 having the amino acid sequence of SEQ ID NO: 43; and
(b) a VL region comprising:
LCDR1 having the amino acid sequence of SEQ ID NO: 46,
LCDR2 having the amino acid sequence of SEQ ID NO: 47, and
LCDR3 having the amino acid sequence of SEQ ID NO: 48.

82. The antibody or antigen binding fragment thereof of claim 80, wherein the VH comprises:
(a) HCDR1 having the amino acid sequence of SEQ ID NO: 60, 61, or 62,
(b) HCDR2 having the amino acid sequence of SEQ ID NO: 63, 64, 65, 66, 67, or 68, and
(c) HCDR3 having the amino acid sequence of SEQ ID NO: 69, 70, 71, or 72.

83. The antibody or antigen binding fragment thereof of claim 80 or 82, wherein the VL comprises:
(a) LCDR1 having the amino acid sequence of SEQ ID NO: 90, 91, 92, 93, 94, or 95,
(b) LCDR2 having the amino acid sequence of SEQ ID NO: 96 or 97, and
(c) LCDR3 having the amino acid sequence of SEQ ID NO: 98, 99, 100, 101, 102, 103, or 104.

84. The antibody or antigen-binding fragment thereof of any one of claims 80-83, wherein:
(a) the VH region comprises:
(i) HCDR1, HCDR2, and HCDR3 having the amino acid sequence of SEQ ID NOs: 41, 53, and 43, respectively;
(ii) HCDR1, HCDR2, and HCDR3 having the amino acid sequence of SEQ ID NOs: 60, 53, and 43, respectively;
(iii) HCDR1, HCDR2, and HCDR3 having the amino acid sequence of SEQ ID NOs: 61, 53, and 43, respectively;
(iv) HCDR1, HCDR2, and HCDR3 having the amino acid sequence of SEQ ID NOs: 62, 53, and 43, respectively;
(v) HCDR1, HCDR2, and HCDR3 having the amino acid sequence of SEQ ID NOs: 41, 63, and 43, respectively;
(vi) HCDR1, HCDR2, and HCDR3 having the amino acid sequence of SEQ ID NOs: 41, 64, and 43, respectively;
(vii) HCDR1, HCDR2, and HCDR3 having the amino acid sequence of SEQ ID NOs: 41, 65, and 43, respectively;
(viii) HCDR1, HCDR2, and HCDR3 having the amino acid sequence of SEQ ID NOs: 41, 66, and 43, respectively;
(ix) HCDR1, HCDR2, and HCDR3 having the amino acid sequence of SEQ ID NOs: 41, 67, and 43, respectively;
(x) HCDR1, HCDR2, and HCDR3 having the amino acid sequence of SEQ ID NOs: 41, 68, and 43, respectively;
(xi) HCDR1, HCDR2, and HCDR3 having the amino acid sequence of SEQ ID NOs: 41, 53, and 69, respectively;
(xii) HCDR1, HCDR2, and HCDR3 having the amino acid sequence of SEQ ID NOs: 41, 53, and 70, respectively;
(xiii) HCDR1, HCDR2, and HCDR3 having the amino acid sequence of SEQ ID NOs: 41, 53, and 71, respectively; or
(xiv) HCDR1, HCDR2, and HCDR3 having the amino acid sequence of SEQ ID NOs: 41, 53, and 72, respectively; and
(b) a VL region comprising:
(i) LCDR1, LCDR2, and LCDR3 having the amino acid sequence of SEQ ID NOs: 46, 47, and 48, respectively;
(ii) LCDR1, LCDR2, and LCDR3 having the amino acid sequence of SEQ ID NOs: 90, 47, and 48, respectively;
(iii) LCDR1, LCDR2, and LCDR3 having the amino acid sequence of SEQ ID NOs: 91, 47, and 48, respectively;
(iv) LCDR1, LCDR2, and LCDR3 having the amino acid sequence of SEQ ID NOs: 92, 47, and 48, respectively;
(v) LCDR1, LCDR2, and LCDR3 having the amino acid sequence of SEQ ID NOs: 93, 47, and 48, respectively;
(vi) LCDR1, LCDR2, and LCDR3 having the amino acid sequence of SEQ ID NOs: 94, 47, and 48, respectively;
(vii) LCDR1, LCDR2, and LCDR3 having the amino acid sequence of SEQ ID NOs: 95, 47, and 48, respectively;
(viii) LCDR1, LCDR2, and LCDR3 having the amino acid sequence of SEQ ID NOs: 46, 96, and 48, respectively;
(ix) LCDR1, LCDR2, and LCDR3 having the amino acid sequence of SEQ ID NOs: 46, 97, and 48, respectively;
(x) LCDR1, LCDR2, and LCDR3 having the amino acid sequence of SEQ ID NOs: 46, 47, and 98, respectively;
(xi) LCDR1, LCDR2, and LCDR3 having the amino acid sequence of SEQ ID NOs: 46, 47, and 99, respectively;
(xii) LCDR1, LCDR2, and LCDR3 having the amino acid sequence of SEQ ID NOs: 46, 47, and 100, respectively;
(xiii) LCDR1, LCDR2, and LCDR3 having the amino acid sequence of SEQ ID NOs: 46, 47, and 101, respectively;
(xiv) LCDR1, LCDR2, and LCDR3 having the amino acid sequence of SEQ ID NOs: 46, 47, and 102, respectively;
(xv) LCDR1, LCDR2, and LCDR3 having the amino acid sequence of SEQ ID NOs: 46, 47, and 103, respectively; or
(xvi) LCDR1, LCDR2, and LCDR3 having the amino acid sequence of SEQ ID NOs: 46, 47, and 104, respectively.

85. The antibody or antigen-binding fragment thereof of any one of claims 80-84, wherein:
the VH region comprises an amino acid sequence having at least 70%, at least 80%, at least 90%, or at least 95% sequence identity to the amino acid sequence of SEQ ID NO: 52, 55, 57, 59, 73, 74, 75, 76, 77, 78, 79, 80, 81, 82, 83, 84, or 85; and
the VL region comprises an amino acid sequence having at least 70%, at least 80%, at least 90%, or at least 95% sequence identity to the amino acid sequence of SEQ ID NO: 87, 89, 105, 106, 107, 108, 109, 110, 111, 112, 113, 114, 115, 116, 117, 118, or 119.

86. The antibody or antigen-binding fragment thereof of any one of claims 80-85, wherein:
the VH region comprises the amino acid sequence of SEQ ID NO: 52, 55, 57, 59, 73, 74, 75, 76, 77, 78, 79, 80, 81, 82, 83, 84, or 85; and
the VL region comprises the amino acid sequence of SEQ ID NO: 87, 89, 105, 106, 107, 108, 109, 110, 111, 112, 113, 114, 115, 116, 117, 118, or 119.

87. The antibody or antigen-binding fragment thereof of any one of claims 80-86, wherein
(a) the VH region comprises the amino acid sequence of SEQ ID NO: 40, and the VL region comprises the amino acid sequence of SEQ ID NO: 45;
(b) the VH region comprises the amino acid sequence of SEQ ID NO: 52, and the VL region comprises the amino acid sequence of SEQ ID NO: 87;
(c) the VH region comprises the amino acid sequence of SEQ ID NO: 55, and the VL region comprises the amino acid sequence of SEQ ID NO: 87;
(d) the VH region comprises the amino acid sequence of SEQ ID NO: 57, and the VL region comprises the amino acid sequence of SEQ ID NO: 87;
(e) the VH region comprises the amino acid sequence of SEQ ID NO: 59, and the VL region comprises the amino acid sequence of SEQ ID NO: 87;
(f) the VH region comprises the amino acid sequence of SEQ ID NO: 52, and the VL region comprises the amino acid sequence of SEQ ID NO: 89; or
(g) the VH region comprises the amino acid sequence of SEQ ID NO: 55, and the VL region comprises the amino acid sequence of SEQ ID NO: 89.

88. The antibody or antigen-binding fragment thereof of claim 87, wherein the VH region comprises the amino acid sequence of SEQ ID NO: 52, and the VL region comprises the amino acid sequence of SEQ ID NO: 87.

89. The antibody or antigen binding fragment thereof of any one of claims 80-88, wherein the antibody or antigen binding fragment thereof is capable of binding siglec-6 with a K_{D} value of between about 10 and about 500 nM, between about 20 and about 100 nM; or between about 25 and about 80 nM.

90. The antibody or antigen binding fragment thereof of any one of claims 80-89, wherein the antibody or antigen binding fragment thereof
(a) is capable of binding human c-Kit;
(b) is capable of binding cynomolgus c-Kit; and/or
(c) has a comparable or lower toxicity compared to a reference antibody capable of binding c-Kit, optionally wherein the reference antibody comprises a VH region comprising the amino acid sequence of SEQ ID NO: 40, and a VL region comprising the amino acid sequence of SEQ ID NO: 45.

91. The antibody or antigen binding fragment thereof of any one of claims 63-90, wherein the antigen binding fragment is a Fab, a F(ab′)2, a Fab′, a single-chain Fv (scFv), an Fv fragment, a Fd fragment, or a diabody.

92. The antibody or antigen binding fragment thereof of any one of claims 63-91, wherein the antibody or antigen binding fragment thereof comprises an antibody heavy chain constant region, optionally wherein the constant region is a human IgG heavy chain constant region, optionally wherein the human IgG heavy chain constant region is an IgG1 heavy chain constant region.

93. The antibody or antigen binding fragment thereof of claim 92, wherein the constant region comprises one or more mutations that increase serum half-life of the antibody or antigen binding fragment thereof.

94. The antibody or antigen binding fragment thereof of claim 92 or 93, wherein the constant region comprises one or more mutations that reduce or abrogate effector function.

95. The antibody or antigen binding fragment thereof of claim 92 or 93, wherein the constant region comprises one or more mutations that enhance effector function.

96. The antibody or antigen-binding fragment thereof of any one of claims 63-95, wherein at least one of the first and second heavy chains is afucosylated (e.g., at least 75% afucosylated).

97. The antibody or antigen binding fragment thereof of any one of claims 63-96, conjugated to a therapeutic agent or a label.

98. A polynucleotide encoding an antibody or antigen binding fragment thereof of any one of claims 1-59 and 63-97.

99. An expression vector comprising the polynucleotide of claim 96.

100. A host cell comprising the polynucleotide of claim 98 or the expression vector of claim 99.

101. The host cell of claim 100, wherein the host cell is a mammalian or insect cell.

102. The host cell of claim 100 or 101, wherein the host cell (a) comprises a Fut8 knockout; and/or (b) overexpresses GnT-III and optionally ManII.

103. A pharmaceutical composition comprising an antibody or antigen binding fragment thereof of any one of claims 1-59 and 63-97, and a pharmaceutically acceptable carrier.

104. A method of treating a disease or disorder in a subject in need thereof, comprising administering to the subject an effective amount of an antibody or antigen binding fragment thereof of any one of claims 1-59 and 63-97, a composition of any one of claims 58-60, or a pharmaceutical composition of claim 103.

105. The method of claim 104, wherein the disease or disorder is a mast cell associated disease or disorder.

106. The method of claim 105, wherein the disease or disorder is an autoimmune disease, an allergy, an inflammation, pruritus, or a cancer.

107. The method of any one of claims 104-106, wherein the disease or disorder is associated with expression of siglec-6 and/or c-Kit.
